(19) United States
(12) Patent Application Publication
(10) Pub. No.: US 2009/0305980 A1
(43) Pub. Date: Dec. 10, 2009

(54) Methods and Compositions Related to TR4

(75) Inventors: Yi-Fen Lee, Rochester, NY (US); Chawnshang Chang, Pittsford, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: UNIVERSITY OF ROCHESTER, Rochester, NY (US)

(21) Appl. No.: 12/300,077

(22) PCT Filed: May 9, 2007
(86) PCT No.: PCT/US2007/011159

§ 371 Date: Aug. 14, 2009

Related U.S. Application Data
(60) Provisional application No. 60/798,974, filed on May 9, 2006.

(51) Int. Cl. A61K 38/17; C12Q 1/68; C12N 15/63; C12N 5/10; A61P 43/00

(52) U.S. Cl. 514/12; 435/6; 435/320.1; 435/325

(57) ABSTRACT

Disclosed are compositions and methods related to TR4 and cancer.

This application claims the benefit of U.S. Provisional Application No. 60/798,974, filed on May 9, 2006, which is incorporated by reference herein in its entirety.

This application was made with government support under federal grants NIH U19 DK62434 awarded by the NIH. The Government has certain rights to this invention.

### I. BACKGROUND

Maintenance of genome integrity is not only important in preventing malignant cell transformation, but also vital to longevity of organisms. Cells are constantly bombarded by environmental insults such as chemicals, ultra-violet (UV) lights, and ionizing (IR), which lead to DNA damage. It has become clear that loss of genome stability due to malfunctions in DNA repair machineries can have catastrophic consequences such as cancers, inheritable diseases, and premature aging. Genomic instability is also a hallmark of aging (Zhivotovsky B, Kroemer G 2004 Nat Rev Mol Cell Biol 5:752-62). Aging is a potent carcinogen, with the incidence of cancer rising exponentially with age. After reaching late-middle age, men face a 50 percent chance of developing cancer and women have a 35 percent chance. Most age-related cancers in humans arise from epithelial cells (DePinho R A (2000) Nature 408:248-54). Older organisms may be less able to cope with accumulated damage and thus be more prone to develop cancer. However, so far, the links between aging and cancer remain largely unknown.

When differentiated cells are irreversibly damaged they can follow one of two pathways: senescence or apoptosis Both mechanisms lead to a loss of functional cells, and elevated apoptosis will finally result in exhaustion of the stem cell pool, and lead to loss of organ cellularity and senescence. Campisi has suggested that senescence represents an example of evolutionary antagonistic pleiotropy, which might prevent tumor formation early in life, but promote carcinogenesis in aged organisms through alterations of tissue microenvironment (Campisi J 2005 Mech Ageing Dev 126:51-8; Parrinello S, et al. 2005 J Cell Sci 118:485-96; Campisi J 2002 Sci Aging Knowledge Environ 2002:pe1; Campisi J 2004 Nat Med 10:231-2). Four recent studies now indicate that premature senescence accompanied by cell cycle arrest occurs in tumors initiated by an oncogenic mutation. Thus, senescence might act as a key tumor suppressor mechanism in the early stage of tumors in vivo (Sharpless N E, DePinho R A (2005) Nature 436:636-7; Chen Z, et al. (2005) Nature 436:725-30; Michalogou C, et al. (2005) Nature 436:720-4; Collado M, et al. (2005) Nature 436:642).

Radiation therapy has played a major role in cancer therapy for many years. It is estimated that in the United States and Europe, more than one million people receive radiation therapy every year as part of their cancer treatment. However, the resistance of tumor cells to radiation limits the success of the radiation therapy. Cellular sensitivity to ionizing radiation is a complicated biological phenomenon that is associated with DNA damage/repair capacity, cell cycle progression, and the execution of apoptosis. Intrinsic radiosensitivity can be altered by the expression of proteins that are involved in the regulation of biological responses upon radiation. Therefore, modulation of expression and activity of the genes involved in DNA repair pathways could control radiation sensitivity.

### II. SUMMARY

The disclosed compositions and methods, in one aspect, relate to TR4 and cancer.

It is to be understood that both the foregoing general description and the following detailed description are exemplary and explanatory only and are not restrictive.

### III. BRIEF DESCRIPTION OF THE DRAWINGS

The accompanying drawings, which are incorporated in and constitute a part of this specification, illustrate several embodiments.

FIG. 1 shows the appearance of TR4 KO and age matched wt mice at 6 months of age. Greasy skin, drooping eye lids, long nails, and hunchback were seen in 6-month-old TR4 KO female mice.

FIG. 2 shows the aged-skin in 6-month-old TR4 KO mice. Reduction of dermal thickness and absence of subcutaneous adipose cells were seen in 6-month-old TR4 KO compared with aged-matched wt mice m, muscle; f, fat; d, dermis; and e, epidermis.

FIG. 3 shows the skeletal abnormalities in aging TR4 KO mice.

Radiography of 3 month (A, B) and 6 month (C, D) TR4 KO and wt mice. TR4 KO mice display curvature of the spinal column (Kyphosis), decreased BMD in both male (N=5) (E), and female (N═5) (F) mice.

FIG. 4 shows the rapid replicative senescence and higher cellular ROS levels in TR4 KO MEFs. A. Cell cycle profile analysis of pPassage 2 (P2) and P4 MEFs from TR4 KO and wt. TR KO display an early G2/M arrest in P4, while wt showed a normal cell cycle distribution. B. The endogenous and H₂O₂-stimulated ROS were measured by flow analysis. KO MEFs have higher cellular ROS levels than wt in both conditions.

FIG. 5 shows the increased DNA single-strand breaks in TR4 KO MEFs. The single strand DNA breaks in TR4 KO wt, and TR4 KO transfected-TR4 were compared by DNA precipitation methods. TR4 KO MEFs display higher percentages of DNA breaks than wt (endogenous and oxidative-stress induced), and TR4-transfected TR4 KO MEFs have reduces the DNA breaks.

FIG. 6 shows TR4 induced TCR repair of UV damaged DNA. FIG. 6A shows CV-1 cells seeded and co-transfected with UV-damaged pRL-SV40 with pCMX-TR4 or pCMX vector control. The intact pGal-SV40 plasmids were used as transfection efficiency control. The LUC activity was normalized by β-gal activity and the relative LUC activity compared to vector control and the mean±SD from triplicate sample was plotted. FIG. 6B shows MEF from TR4KO vs wt were exposed to UV and RNA synthesis recovery rate were measured at the indicated time. FIG. 6C shows the TCR-pathway related gene profiling between KO (black bar) vs wt (open bar) were analyzed by Q-PCR.

FIG. 7A shows that H₂O₂ and IR induce TR4 mRNA expression. Wt MEFs were treated with 200 μM of H₂O₂ and 6Gys IR, and cells were harvested 2 h after H₂O₂, or 4 h, 12 h, and 24 h post-IR. The levels of TR4 mRNA were measured, and relative expression level was calculated by setting the untreated as 1. FIG. 7B shows that stress induces TR4 protein expression. H1299 cells were treated with 250 μM H₂O₂ for 2 hrs, or 6Gys IR, and 100 J/m2 UV, and then were harvested at indicated times. TR4 protein expressions were analyzed by Western blotting with a specific mouse monoclonal antibody against TR4 (#15).

FIG. 8 shows subcellular localization of TR4 upon the stress stimulation. Cells were treated with 250 μM H₂O₂ and then change to culture medium. 4 h post-treatment cells were fixed and incubated with TR4 #15 antibody, and then FITC conjugated secondary antibody, and the cellular localization was observed under fluorescence microscopy.

FIG. 9 shows that TR4 regulates Gadd45a gene activity. FIG. 8A shows the reduced Gadd45a expression in 3 m, and 6 m TR4 KO muscle, and reduced SIRT1 in 6 m old TR4 KO when compared with wt. FIG. 8B shows that TR4 activates Gadd45a promoter containing reporter genes (GaddLuc), not deletion reporter (GaddLuc3). CV-1 cells were co-transfected with Gadd45 reporter and different amounts of TR4 (PCMX-TR4), and Luc activities were measured.

FIG. 10A shows that TR4 KO MEFs are more sensitive to H₂O₂ treatments. MEFs from wt and TR4 KO were seeded and then after 24 h treated with 50-200 μM H₂O₂ for 2 h, and cells were harvested 72 h after treatment for MTT assay. The % of cell survival was calculated by comparing with untreated cells. FIG. 10B shows TR4 induced repair of UV-induced DNA damage. C2C12 cells and TR4 KO MEFs were seeded and co-transfected with UV-damaged SV40 renilla with pCMXTR4 or pCMX vector control. SV40 gal was used as transfection efficiency control. Luciferase activities were measured 2 days after transfection and Luc activity was normalized by β-gal activity.

FIG. 11 shows the loss of vitamin E anti-ROS effects in TR4KO MEFs. MEFs from wt and TR4 KO were treated with 200 μM H₂O₂ for 30 min in the presence/absence of 100 nM of α-tocopherol, and the cellular ROS levels were then measured by flow cytometric analysis. The relative ROS levels were calculated by comparing with untreated MEFs from wt and TR4 KO independently.

FIG. 12 shows TR45′-promoter analysis. FIG. 11A shows an illustration of the putative transcriptional factors located in the TR45′-promoter. FIG. 11B shows the basal transcriptional level tests in TR45′-promoter containing luciferase. Serial deletions of TR45′-promoter have been constructed according to the available enzymes and then transfected into CV-1 cells. The basal transcriptional activities were assayed by luciferase.

FIG. 13 shows that two clones of TR4 RNAi suppress TR4-mediated TR4RE-luc activity. CV-1 cells were cotransfected with pCMX-TR4, hTR4-siRNA 1-4, and 2-9 and TR4RE/ApoE (HCR-1-Luc) with different ratios as indicated, and then luciferase activities were measured 48 h after transfection.

FIG. 14 shows that TR4 induced repair of UV-induced DNA damage, and phosphorylation of TR4 affects DNA repair capacity. C2C12 cells were seeded and co-transfected with UV-damaged SV40 renilla with pCMXTR4 or pCMX vector control, and several TR4 phosphorylation mutants. SV40 gal was used as transfection efficiency control. Luciferase activities were measured 2 days after transfection and Luc activity was normalized by β-gal activity.

FIG. 15 shows that TR4 induces NHEJ. CV1 cells were seeded and co-transfected with GFP-Pem-Ad2 (NHEJ substrate) plasmid which is digested by HindIII and and μg of pDsRed with pCMX vector control (A) or pCMXTR4 (B). Area 2 shows the percentage of cells staining positive with both GFP and dsRED, indicating NHEJ efficiency.

FIG. 16 shows the examination of suppression of TR4-mediated transaction activity by on TR4 RNAi. CV-1 cells were co-transfected with pCMXTR4, two TR4 responsive Luc reporters (PEPCK-49-luc and DR1×3Luc) and pRetro-TR4-RNAia, b, and c. and then luciferase activities were measured 48 h after transfection.

FIG. 17 shows a pathological examination of ventral prostate. VP from 17 month old TR4 KO and wt littermates were collected, formalin fixed, and processed for HE staining. The photos were taken under 40× magnification.

FIG. 18 shows the elevation and abnormal TR4 expression during prostate cancer progression in TMA analyses. A. A typical example of nuclear staining of TR4 on normal prostate, B. A typical example of nuclear staining of TR4 on HGPIN. C. A typical example of cytoplasm staining of TR4 on LG tumor. D. A typical example of cytoplasmic staining of TR4 HG tumor sample.

FIG. 19 shows that total TR4 and cytoplasmic TR4 signals were increased with prostate cancer progression from benign, PIN. LG, and HG cancers by TMA prostate cancer analyses.

FIG. 20 shows that TR4 KO MEF are more sensitive to IR. MEF from both TR4 KO and wt were exposed to gamma irradiation at the 3, 6, and 9 Gys, and the cells were measured by MTT assay at 6-dat post-IR. The survival cells were calculated as the ratio to non-IR cells. These data were generated from three different independent experiments.

FIG. 21 shows that IR induces TR4 mRNA and protein expression. Wt MEFs and H1299 cells were treated with 6Gys IR, and cells were harvested Oh, 4 h, and/or 8 h, 12 h, and 24 h post-IR. The levels of TR4 mRNA were measured by real-time PCR, and relative expression level was calculated by setting the untreated as one. Western blotting with a specific mouse monoclonal antibody against TR4 was used to determine TR4 protein expression.

FIG. 22 shows that UV induces TR4 mRNA and protein expression. Wt MEFs and H1299 cells were treated with 6Gys IR, and cells were harvested Oh, 4 h, and/or 8 h, 12 h, and 24 h post-IR. The levels of TR4 mRNA were measured by real-time PCR, and relative expression levels were calculated by setting the untreated control as one. Western blotting with a specific mouse monoclonal antibody against TR4 was used to determine TR4 protein expression.

FIG. 23 shows an illustration of putative phosphorylation site on TR4 by computer program at MIT (http://scansite.mit.edu)

FIG. 24 shows that the phosphorylation of TR4 at Ser-144 and Ser-351 regulates UV-damaged DNA repair. CV-1 cells were seeded and co-transfected with UV-damaged SV40 renilla with pCMXTR4, pCMX vector control, or pCMXTR4S144A, pCMXTR4S144D, pCMXTR4S351A, or pCMXTR4S351. SV40 gal was used as transfection efficiency control. Luciferase activities were measured 2 days after transfection and Luc activity was normalized by β-gal activity.

FIG. 25 shows that the expression of CSB was reduced in the absence of TR4. Total RNA from 5 weeks old TR4 KO and WT muscle, and TR4 WT and KO MEF cells were extracted; the levels of TR4 mRNA were measured by real-time PCR, and relative expression level was calculated by setting the WT as one.

FIG. 26 shows the characterization of overexpression and knockdown of TR4 in LNCaP. A. Nuclear localization of TR4 in LNCaP cells. B. Q-PCR to qualify TR4 mRNA in pBabe, pBabe-TR4, Scramble and TR4 RNAi-stable clones. C. Cell growth of those four clones by MTT assay. Western analysis of TR4 protein expression of each clone is shown in left upper panel.

FIG. 27 shows protein expression profiles of TR4 and associated complex from PCa patients.

FIG. 28 shows the characterization of EGFP-TR4. The NLS amino acid sequence in TR4 and TR4NLS mutations are illustrated. The subcellular localization of EGFP TR4 wt, TR4mt were examined in PC-3 cells. The transcriptional activity of EGFP-TR4 wt, and -TR4mt were tested by measuring the PEPCK-Luc activity after transfection of EGFP TR4 constructs.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Abbreviations: CAT, chloramphenicol acetyltransferase; DBD, DNA-binding domain; E2, 17β-estradiol; ER, estrogen receptor; ERE, estrogen response element; GST, glutathione S-transferase; LBD, ligand-binding domain; PR, progesterone receptor; TR2, Testicular orphan receptor 2, TR4, Testicular orphan receptor 4; RA, retinoic acid; PPARα, peroxisome proliferator-activated receptor a; CAT, chloramphenicol acetyltransferase; RAR, retinoic acid receptor; PPRE, peroxisome proliferator response element; 1,25-(OH)₂D3, 1,25-dihydroxyvitamin D₃; Kd, equilibrium dissociation constant, TR4 associated constant; AR, androgen receptor; GR, glucocorticoid receptor; TR, thyroid hormone receptor; TR4RE, TR4 response element; TR4-N, TR4-N terminus; TR4-DL, TR4 DNA binding domain (DBD) and ligand binding domain (LBD); DR, direct repeat; HDACs, histone deacetylases; TSA, Trichostatin; EMSA, electrophoretic mobility shift assay; LUC, luciferase; -UL, minus uronolactone.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between “10” and “15.” It is also understood that each unit between two particular units are also disclosed. For example, if “10” and “15” are disclosed, then “11,” “12,” “13,” and “14” are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “decrease” can refer to any change that results in a smaller amount of a composition or compound, such as TR4, activity. Thus, a “decrease” can refer to a reduction in an activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a composition or compound, such as TR4, activity. Thus, for example, an increase in the amount in Tr4 activity can include but is not limited to a 10%, 20%, 30%, 40%, 50%, 60%, 70%, 80%, 90%, or 100% increase.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

“Obtaining a tissue sample” or “obtain a tissue sample” means to collect a sample of tissue from a subject or measure a tissue in a subject. It is understood and herein contemplated that tissue samples can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, aspiration, tissue swab, spinal tap, magnetic resonance imaging (MRI), Computed Tomography (CT) scan, Positron Emission Tomography (PET) scan, and X-ray (with and without contrast media).

Transcription activity as used herein refers to the activity a particular protein has as an activator of transcription. There are many ways that this activity can be determined, for example, CAT assays or luceriferase assays are two examples used herein.

A system refers to a collection of components which have a certain function or activity. For example, a cell that is transfected with a particular nucleic acid that is expressed can be a system that can be used for the expression of the cognate nucleic acid.

“Interacts” means that two (or more) molecules touch one another in a way beyond the touching that takes place because of random contacts between molecules. “Interacts” can be thought of as “binding” between two or more molecules, and therefore can have dissociation and association constants as well as equilibrium constants.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TR4 is disclosed and discussed and a number of modifications that can be made to a number of molecules including the TR4 is discussed, specifically contemplated is each and every combination and permutation of TR4 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. COMPOSITIONS AND METHODS

The aging process is a unique feature of the life cycle of all multicellular organisms with progressive impairment, ultimate failure in homeostasis maintenance, and resultant death (Hasty, P., Campisi, J., Hoeijmakers, J., van Steeg, H., and Vijg, J. Science, 299: 1355-1359, 2003.) The accumulation of somatic damage is a main cause of the aging process. Among the various sources of somatic damage, reactive oxygen species (ROS), the natural by-products of oxidative energy metabolism in mitochondria, are considered as the ultimate cause of aging (Droge, W. Adv Exp Med Biol, 543: 191-200, 2003).

DNA damage is a common cell death-inducing signal but the death program that is activated varies by cell type. DNases and ROS can damage DNA. The mitochondrion is a significant source of ROS that are associated with the pathogenesis of many diseases and with aging (Genova et al., Ann N Y Acad Sci, 1011: 86-100, 2004; Huang et al. Front Biosci, 9: 1100-1117, 2004.) The oxygen species that are typically linked to oxidative stress include superoxide anion, hydroxyl radical (OH), hydrogen peroxide (H2O₂), nitric oxide (NO) and peroxynitrite (ONOO—). Although generation of these species from molecular oxygen is a normal feature of mammalian respiration, ROS directly targets DNA resulting in different lesions, such as single- or double-strand DNA breaks (Bohr, V. A., Stevnsner, T., and de Souza-Pinto, N. C. Gene, 286: 127-134, 2002). The most frequent oxidative damage to DNA is the 8-hydroxylation/oxidation of guanine base to 8-hydroxydeoxguanosine (8-OHdG). These lesions disrupt vital processes, such as transcription and replication, which can cause growth arrest or cell death. To cope with DNA damage, organisms evolved an intricate network of DNA damage repair pathways, each focusing on a different class of lesion (Lehmann, A. Curr Biol, 12: R550-551, 2002). Alterations in the genome have been considered critically important. In addition to DNA damage, ROS can cause severe damage to cellular proteins and lipids when produced at high levels by disease processes such as ischemia, atherosclerosis, diabetes, pulmonary fibrosis, neurodegenerative disorders, and arthritis.

The mitochondria theory of aging (MTA) postulates that damage to mitochondrial DNA (mtDNA) and organelles by ROS leads to loss of mitochondrial function and loss of cellular energy (Jacobs, H. T. Aging Cell, 2: 11-17, 2003). Mutations in mtDNA occur at 16 times the rate seen in nuclear DNA. Unlike nuclear DNA, mtDNA has no protective histone proteins and DNA repair is less efficient in mitochondria than in the nucleus (Mandavilli, B. S et al. Mutat Res, 509: 127-151, 2002). Free radicals leaking from mitochondria result in damage to cellular protein, lipid, and DNA throughout the cell. This damage has been implicated as a cause of aging. mtDNA deletion mutations accumulate in post-mitotic cells with age. The inefficient mitochondria survive and reproduce causing the animal to develop early onset of senescence.

Mouse models have shown that accelerated aging is a consequence of defects in genome maintenance systems. TTD mutant mice, which have deficiencies in DNA repair and gene transcription, have developed a premature aging process (de Boer et al. J. H. Science, 296: 1276-1279, 2002.)

p53 deletion mutant mice display an early onset of phenotypes associated with aging (Tyner et al. Nature, 415: 45-53, 2002) and the Ku 80 deficient mice, which have an impairment in double-strand DNA break repair system, also developed early onset of senescence (Vogel et al. Proc Natl Acad Sci USA, 96: 10770-10775, 1999; Parrinello et al. J. Nat Cell Biol, 5: 741-747, 2003.) Hence, the accelerated aging syndromes in mice with genetic defects in genome maintenance show that genome instability, driven by oxidative damage, is a primary cause of normal aging. Since defects in genome maintenance lead to accelerated aging in human and mice, it appears that normal aging is caused by inadequately repaired DNA damage. Genotype-phenotype correlations in mouse models of defects in genome maintenance can provide valuable insights into basic mechanisms of aging and natural defense systems that promote longevity. In addition to DNA repair gene mutation, deletion of klotho and SNF2-like gene PASG develops premature aging Kuro-o et al. Nature, 390: 45-51, 1997; et al. Genes Dev, 18: 1035-1046, 2004.) The premature aging phenotype found in mice in defective mtDNA polymerase further shows the role of mitochondria in maintaining the longevity. In addition to mice, several human diseases exhibit symptoms of acceleration of aging. Diseases that resemble certain aspects of accelerated aging are known as segmental progerias, because of segments of aging in each disease condition. Segmental progerias include disease of DNA-damage/repair (such as Werner's syndrome (Bohr et al. Biogerontology, 3: 89-94, 2002) and xeroderma pigmentosum), and diseases showing telomere abnormalities (such as Hutchinson-Gilford syndrome and Down's syndrome)(Brown, W. T. Curr Probl Dermatol, 17: 152-165, 1987; Martin, G. M. Natl Cancer Inst Monogr, 60: 241-247, 1982; Brown, W. T. Annu Rev Gerontol Geriatr, 10: 23-42, 1990).

Disclosed herein are compositions comprising TR4 and vectors encoding a TR4 gene. It is understood and herein contemplated that the TR4 vectors disclosed herein can comprise a promoter operably linked to a TR4 gene. It is further understood that the promoters disclosed herein can be stress responsive promoters and comprise cis-acting stress responsive elements (SRE). Thus disclosed herein are vectors comprising a TR4 gene, further comprising a promoter, wherein the promoter comprises a stress response element, and wherein the promoter is operably linked to the TR4 gene. Further disclosed are compositions comprising the vectors disclosed herein. Also disclosed herein are cells comprising the TR4 vectors disclosed herein. It is understood and herein contemplated that the disclosed vectors and compositions can be used to treat a disease. Thus, disclosed herein are methods of treating a disease in a subject comprising administering to the subject TR4. Also disclosed are methods of treating a disease in a subject comprising administering to the subject a vector comprising a TR4 gene. Also disclosed are methods of treating a cancer in a subject comprising administering to the subject TR4. Also disclosed are methods of treating a cancer in a subject comprising administering to the subject a vector comprising a TR4 gene. It is also understood that a subject can be a cell, mammal, mouse, rat, pig, dog, cat, cow, horse, monkey, chimpanzee or other none human primate, or human.

The TR4 is a member of the nuclear receptor superfamily. The nuclear receptor superfamily is comprised of transcription factors that are related by sequence and structure, yet are specifically induced or repressed by a wide variety of chemical compounds. Functioning as transcription factors, nuclear receptors can control the expression of target genes and thereby direct developmental, physiological, and behavioral responses from the cellular level to that of the whole organism (Beato, M. Faseb J, 5: 2044-2051, 1991; Beato, M. and Klug, J. Hum Reprod Update, 6: 225-236, 2000). The structural features common to nuclear receptors include those required for ligand binding, dimerization, DNA binding, and transactivation. Binding of a particular receptor to a specific DNA sequence or hormone response element (HRE) within the promoter of one of its target genes is mediated by a DNA binding domain that contains two zinc finger motifs. This DNA binding domain (DBD) displays a high level of amino acid homology between nuclear receptors and has been used as a template when developing probes with which to screen for new members of the nuclear receptor family. Using this strategy, many structurally related receptors have been identified, yet remain a mystery in terms of their specific ligands and/or their physiological functions, and are therefore referred to as orphan receptors. In vitro studies suggest that TR4 functions as a master regulator to modulate many signaling pathways, including maintenance of erythrocyte progenitor populations in the human erythropoietin gene (EPO) (Kim E, et al. (2003) J Biol Chem 278:46919-46926), modulating neurogenesis, via ciliary neurotrophic factor alpha (CNTFRα) (Young W J, et al. (1997) J Biol Chem 272:3109-3116; Young W J, et al. (1998) J Biol Chem 273:20877-20885), interfering with retinoic acid/RAR/RXR (Lee Y F, et al. (1998) J Biol Chem 273:13437-13443), thyroid hormone/T₃R (Lee Y F, et al. (1999) J Biol Chem 274:16198-16205), vitamin D 3/VDR, AR (Lee Y F, et al. (1999) Proc Natl Acad Sci USA 96:14724-14729) and ER-mediated pathways, and facilitating viral infection and propagation of HPV-16 and SV40 (Lee H J, et al. (1995) J Biol Chem 270:30129-30133). To our surprise, TR4 KO mice developed accelerated aging syndromes at 5-6 month of age, and TR4 KO derived mouse embryonic fibroblasts (MEF) also displayed an early onset cell growth arrest.

Both embryonic and adult tissue distribution analysis demonstrated that TR4 is expressed mainly in neural and testis during embryonic development. In situ hybridization experiments using TR4 specific probes have shown transcripts present in actively proliferating cell populations of the brain and peripheral organs during embryonic development. The expression of TR4 at sites of sensory innervation and in sensory organs throughout embryogenesis indicates an important role for these receptors in this critical aspect of nervous system development. Additionally, high expression of TR4 in the developing brain and spinal cord, including specific expression in motor neurons, show that these receptors can be involved in the proper development of movement and limb coordination (Young et al. J Biol Chem, 272: 3109-3116, 1997).

TR4 is closely related to the retinoic X receptor (RXR), and binds to AGGTCA DNA sequence motifs in direct repeat orientation, with variable spacing, in the promoters of its target genes (Chang et al. Proc Natl Acad Sci USA, 91: 6040-6044, 1994). Therefore, TR4 can directly influence gene activation by directly binding to DNA and activating genes such as ApoE and Vitamin D receptor (VDRE) (Kim et al. J Biol Chem, 278: 46919-46926, 2003; Lee et al. J Biol Chem, 274: 16198-16205, 1999). On the other hand, TR4 acts as a suppressor to influence other receptor functions, such as RXR/retinoic acid receptor (RAR), androgen receptor (AR), and estrogen receptor (ER) (Lee et al. J Biol Chem, 273: 13437-13443, 1998; Lee et al Proc Natl Acad Sci USA, 96: 14724-14729, 1999; Shyr et al. J Biol Chem, 277: 14622-14628, 2002) by competition for the same DNA binding sites or through protein-protein interactions.

In vitro data show that TR4 functions as a master regulator to modulate many signaling pathways. To investigate TR4 function, mice lacking TR4 (TR4 KO) via targeted gene disruption have been created (Collins et al. Proc Natl Acad Sci USA, 101:15058-15063, 2004, herein incorporated by reference in its entirety for its teaching concerning TR4 KO mice). The lambda KOS system was used to derive a TR4 targeting vector, and three independent genomic clones spanning exons 4-10 were isolated. The targeting vector was derived from one clone and contained a 2173 bp deletion that included most of exon 4 and all of exon 5. The genomic sequence encoding the DBD of TR4 was replaced by a Lac-Z/Neo selection cassette. The Not I linearized vector was electroporated into strain 129SvEvbrd (LEX1) embryonic stem (ES) cells, and G418/FIAU-resistant ES cell clones were isolated and screened by Southern blot for homologous recombination of the mutant DNA. One targeted ES cell clone was injected into blastocysts of strain C57BL/6 (albino), which were then inserted into pseudopregnant female mice for continuation of fetal development. Resulting chimeric male mice were then mated to C57BL/6 (albino) females to generate animals heterozygous for the mutation. The TR4 KO mice demonstrate high rates of early postnatal mortality, as well as significant growth retardation. TR4 KO mice also display reproductive defects, in which reduced fertility was seen in both genders (Mu et al. Mol Cell Biol, 24: 5887-5899, 2004).

The surviving adult TR4 KO mice develop growth impairments, including growth retardation, hypoglycemia, and mild late-onset myopathy where mitochondria-like proliferation inclusions were found. Furthermore, decline of mitochondria function is often linked to aging related syndrome (Roubertoux et al. Nat Genet, 35: 65-69, 2003). By 6 months, most of the mice develop kyphosis and a sign of osteoporosis with a reduced bone mineral density (BMD). A premature ovarian failure was observed in three 6 month-old TR4 KO females, in which there was no active estrus cycle and complete anovulation. All those phenotypes indicate TR4 KO mice develop premature aging. TR4 KO mice embryonic fibroblast (MEF) cells display a dramatic reduction in replicative lifespan. Emerging late age-onset phenotypes observed in TR4 KO mice, abnormal mitochondria proliferation, and reduction of MEF replicative lifespan show that TR4 plays an important role in maintaining the genome stability, and loss of TR4 in mice can lead to development of systemic problems which cause the premature aging process.

As discussed above, TR4 KO mice, in general, have shorter life spans, and most of the mice won't live over one year. TR4 KO mice also have high pre-puberty mortality with a 35% mortality rate. The surviving adult KO mice develop growth impairments, including growth retardation, hypoglycemia, and mild late-onset myopathy where mitochondria-like proliferation inclusions were found. Decline of mitochondria function is often linked to aging related syndrome (Martin et al. Nature, 429: 417-423, 2004; Stevnsner et al. Exp Gerontol, 37: 1189-1196, 2002.) By 6 months, most of the mice develop kyphosis and a sign of osteoporosis with a reduced bone mineral density (BMD). A premature ovarian failure was observed in three 6 month-old TR4 KO females, in which there was no active estrus cycle and complete anovulation. All above phenotypes indicate TR4 KO mice develop premature aging. TR4 KO mouse embryonic fibroblast (MEF) cells display a dramatic reduction in replicative lifespan. Emerging late age-onset phenotypes observed in TR4 KO mice, abnormal mitochondria proliferation, and reduction of MEF replicative lifespan shows that TR4 plays an important role in maintaining the genome stability and loss of TR4 in mice that can lead to development of systemic problems that cause premature aging.

TR4 KO mice developed an early onset of aging progression, which provides a model to study the initiation and progression of the aging process through monitoring the changes of multiple organ systems throughout the life span. Determination of the stages, as well as gender differences, and organs which are targeted by aging is necessary to dissect the mechanisms that are associated with the premature aging process in TR4 KO mice. Most importantly, the changes between earlier stage vs. later stages in particular organs/systems during this aging process can be used to identify factors operating in early or mid-life origins and consequences that occur in late stage, all of which are essential for understanding the aging process. Many organs and systems can be examined. Examples include skin, muscle, bone, cardiovascular function, urinary function, reproductive systems, and immune systems through all segments of the life span, from neonatal (P7), before puberty (1 month), young adulthood (2-3 month), mid-age (4-6 month), mid-late (7 month to 1 yr), to late-life (over 1 year).

As discussed above, progressive decline in mitochondria function accompanies aging. One of the theories of mitochondria aging (MTA) is that reactive oxidative species (ROS), natural by-products of oxidative energy metabolism in mitochondria, which directly target DNA result in different lesions (Ames et al. J Alzheimers Dis, 6: 117-121, 2004; Liu et al. Ann N Y Acad Sci, 959: 133-166, 2002; Ames et al. Ann N Y Acad Sci, 1019: 406-411, 2004). The burden of ROS is largely counteracted by antioxidant defense and DNA repair systems, with inadequately repaired DNA damage eventually leading to aging. In young organisms, there are a large number of small mitochondria that provide needed ATP, however there are many large mitochondria in aged organisms. These larger mitochondria are not as bio-energetically efficient as the youthful, normal, small mitochondria (Bertoni-Freddari et al. Ann N Y Acad Sci, 717: 137-149, 1994; Miquel, J. Exp Gerontol, 33: 113-126, 1998; Lee et al. J Steroid Biochem Mol Biol, 81: 291-308, 2002.) Electron microscopy examination of skeletal muscle from 6 month old TR4 KO showed enlarged and abnormal proliferation mitochondria, an indication of mitochondrial functional decline. TR4 KO mice that have mitochondrial dysfunction generated excess ROS burden to induce DNA damage. Furthermore, the impairment of DNA repair capacity eventually results in accelerated aging in TR4 KO mice. Mitochondria function, mitochondrial DNA integrity, ROS status, and DNA damage can be examined in different stages of TR4 KO mice, for comparisons with their wild type littermates.

MEF rapid senesce is the result of severe oxidative stress which induces extensive DNA damage and/or chromosomal aberrations and is a landmark of aging cells (Davis et al. J Cell Sci, 116: 1349-1357, 2003). TR4 KO MEF cells display a rapid senescence, at which TR4 KO MEF cells arrest at G2/M phase after four population doublings (P4), indicating that TR4 KO MEF cells fail to overcome replicative senescence that is caused by oxidative stress. MEF cells derived from TR4 KO and wild type mice can be examined to determine the mechanisms underlying the replicative senescence and determine its contribution to accelerated aging in mice. MEF cells are challenged with DNA-damage inducers, such as hydrogen peroxide (H2O₂) and UV, and then ROS status, the degree of DNA damage, DNA repair ability, DNA replication, and cell survival can be measured and compared. Viral TR4 infection is used to rescue the defects in TR4 KO MEF to confirm the roles of TR4. The known genes related to the stress-response, cell survival, and DNA damage/repair systems are compared between TR4 KO and wt MEF cells. In addition, microarray analysis can be used to identify the TR4 targeted genes, which are responsible for the TR4 KO MEF rapid replicative senescence.

It is understood that TR4 activity can be modulated by phosphorylating or dephosphorylating serines of TR4. For example, TR4 activity increases when TR4 is phosphorylated at the serine at position 144 (S144) or dephosphorylated at the serine at position 351 (S351). Thus, contemplated herein are mutant TR4 molecules wherein the mutant TR4 contains a substitution at position 144 and/or position 351, wherein the substitution phosphorylates or dephosphorylates TR4. Thus, for example, the mutant can comprise a substitution of serine for aspartic acid at position 144 (S144D). Another example of a mutant TR4 is a dephosphorylated mutant, wherein the mutant TR4 comprises a substitution of alanine for serine at position 351 (S351A). The mutant can be constitutively expressed or under the control of an inducible promoter. Thus disclosed herein are any of the methods of treatment using TR4, wherein the TR4 is a mutant comprising a substitution of aspartic acid for serine at position 144 (S144D). Also disclosed are any of the methods of treatment using TR4, wherein the TR4 is a mutant comprising a substitution of alanine for serine at position 35.1 (S351A).

It is understood that the disclosed vectors and compositions can be used in combination with other molecules and agents that can increase TR4 activity and effectiveness. Thus, disclosed herein are compositions comprising vectors comprising a TR4 gene, wherein the composition further comprises an agent that phosphorylates TR4 at the serine at position 144 (S144) of TR4. Also disclosed are compositions comprising vectors comprising TR4, further comprising an agent that dephosphorylates TR4 at the serine at position 351 (S351) of TR4. It is understood that an agent that phosphorylates TR4 at one residue may also dephosphorylate TR4 at another residue. Thus, disclosed herein are compositions comprising a TR4 vector, further comprising an agent that phosphorylates TR4 at the serine at position 144 (S144) and dephosphorylates TR4 at the serine at position 351 (S351).

### C. METHOD OF TREATING CANCER

### 1. Prostate Cancer and Aging

The incidence of epithelial cancers, including prostate, colon, and breast increases exponentially with age (Hasty P, et al. (2003) Science 299:1355-1359). Prostate cancer is the one of most common cancer in men, and the second leading cause of cancer related death among men in the United States. The single greatest risk factor for prostate cancer is aging. Senescence-associated changes in the prostate, including cumulative mutations, loss of scavenger surveillance of oxidative stresses, telomere dysfunction, chronic inflammation, decreased response to apoptotic signals, and alterations in tissue microenvironment, are believed to play important roles in the genesis of prostate cancer (Bavik C, et al. (2006) Cancer Res 66:794-802). However, how aging increases the prostate susceptibility to cancer, and what genetic events during the aging process contribute to prostate pathological alterations, remain largely unknown. Therefore, it is important to identify these genetic events involved in promoting prostate cancer cell growth and progression during the aging process, and thus might lead to development of new prognostic markers and new therapeutic strategies.

### 2. Anti-Cancer Barrier:

DNA damage response (DDR), and cellular senescence: Cells are constantly bombarded by genotoxic stress from cell-intrinsic sources, such as replication errors and ROS as well as by environmental insults, such as chemicals, UV lights, and ionizing radiation (IR), which lead to DNA damage. It has become clear that loss of the genome stability due to malfunctioned DNA repair machineries can have catastrophic consequences that lead to tumorigenesis (Hasty P, et al. (2003) Science 299:1355-1359). Given the importance of these DNA repair pathways in defending genome integrity, it is not surprising that mutation of genes in these pathways lead to serious diseases including cancer. Recent reports demonstrate that DDR is up-regulated during the early stage of tumor development and serves as a candidate barrier for tumorigenesis (Bartkova J, et al. (2005) Nature 434:864-870). In these studies, the double strand DNA break downstream signaling ATM-Chk2-p53 pathway is constitutively activated in bladder, breast, and colon cancers starting from early stages of development.

ROS induced DNA damages and activation of oncogenes can cause cells to enter senescence, an irreversible cell arrest stage, to protect cells from further damages (Ben-Porath I, Weinberg R A (2004) J Clin Invest 113:8-13; Ben-Porath I, Weinberg R A (2005) Int J Biochem Cell Biol 37:961-976; Finkel T, Holbrook N. J. (2000) Nature 408:239-247). Therefore, senescence is a major barrier against malignant transformation and the barrier of senescence has to be overcome to continue cell proliferation indefinitely, which is a prerequisite for tumor formation (Kim E, et al. (2003) J Biol Chem 278:46919-46926). However, organisms have fairly constant numbers of cells; the accumulation of senescent cells might compromise tissue renewal or repair ability, so cell senescence promotes aging. The fact that senescence limits the proliferation of cells at risk of malignant transformation suggests that aging is, at least in part, a consequence of the tumor suppressor mechanism that prevents the cells harboring dangerous mutations from turning into fully fledged cancer (Hasty P, et al. (2003) Science 299:1355-1359; Sharpless N E, DePinho RA (2005) Nature 436:636-637).

### 3. Molecular Pathology, Detection, Prognostic Markers of Prostate Cancer

The molecular pathology of prostate cancer is complex; it is a heterogenous disease ranging from asymptomatic to a rapidly fatal systemic malignancy (Mimeault M, Batra SK (2006) Carcinogenesis 27:1-22). The therapies for patients with prostate cancer include prostatectomy, radiation, chemotherapy, and hormonal therapy (Kasamon K M, Dawson NA (2004) Curr Opin Urol 14:185-193), and prostate-specific antigen (PSA) is used to monitor the treatment. For more than a decade, PSA has been extensively used as a biomarker to screen for prostate cancer and is also used as a surrogate marker to assess response to therapy for prostate cancer. It is a protein product produced by both normal and cancerous prostate cells. In addition, Gleason grading on histopathological examination is the best prognostic indicator of prostate cancer to date; however interobserver variations and sampling discrepancies do occur and morphologically identical prostate cancer might behave differently. The ability to predict biochemical recurrence and cancer progression after therapies using clinical and pathological variables has been extensively investigated. Identifying which patients are at highest risk for recurrence, and which types of patients have better treatment outcomes are important. Serum PSA level is widely accepted as being helpful in diagnosis of evaluation and in screening men for recurrence. Despite the widespread use of PSA as screening tool for prostate cancer, the clinical significance of elevated PSA values is still debated. PSA cannot give satisfactory prediction of disease progression or survival, and there are still discrepancies between the levels of PSA, with cancer diagnosis, and treatment prognosis. Therefore, an appropriate evaluation of disease status and treatment efficacy is also required. It is important to understand the genetic events involved in prostate cancer cell growth and progression, and is the first step towards improving treatment outcomes.

The disclosed compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer; genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. It is also understood that the disclosed treatments can be used to treat any known cancer. Thus, for example, it is understood that the disclosed treatments can be used to treat prostate cancer.

One method used in the art to treat cancer is irradiation of the subject. However, TR4 activity can result in a subject resistant to radiation treatment. Thus, disclosed herein are methods of increasing the efficacy of radiation treatment for a subject comprising administering to the subject an agent that inhibits TR4. It is understood and herein contemplated that the agent can be administered locally to treat only the cancer cells. Therefore, disclosed herein are methods of increasing the efficacy of radiation treatment for a subject comprising administering to the subject an agent that inhibits TR4 in cancer cells. The agent that inhibits TR4 can be any agent that will block TR4 activity such as an antibody of siRNA. Thus, disclosed herein are methods of increasing the efficacy of radiation treatment for a subject comprising administering to the subject an agent that inhibits TR4, wherein the agent is an anti-TR4 antibody or anti-TR4 siRNA.

It is understood that the disclosed treatment methods can be used in combination with other recognized treatments of cancer. Thus disclosed herein are methods of treating a cancer in a subject comprising administering to the subject a composition comprising a low molecular weight antioxidant (LMWA) and TR4. Thus, disclosed herein are methods of treating prostate cancer in a subject comprising administering to the subject a low molecular weight antioxidant and TR4. It is further understood that the TR4 can be expressed in a vector. Thus, disclosed herein are compositions comprising a LMWA and a vector comprising TR4. Also disclosed are methods of treating prostate cancer in a subject comprising administering to the subject a low molecular weight antioxidant and a vector comprising TR4.

a) Antioxidants

The compositions disclosed herein can also comprise other molecules. For example, disclosed herein are compositions comprising TR4 vectors, wherein the composition further comprises a low molecular weight antioxidant. Generally, antioxidants are compounds that get react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The compositions can contain any antioxidants, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly .alpha.-tocopherol, etc., vitamin E succinate, and trolox (a soluble Vitamin E analog) Vitamin C (ascorbic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavonoids and flavonoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

Flavonoids, also known as “phenylchromones,” are naturally occurring, water-soluble compounds which have antioxidant characteristics. Flavonoids are widely distributed in vascular plants and are found in numerous vegetables, fruits and beverages such as tea and wine (particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof and quercetin). Thus, disclosed herein are compositions comprising TR4 vectors, wherein the composition further comprises a low molecular weight antioxidant (LMWA), wherein the LMWA is selected from the group consisting of Vitamin E, Vitamin C, selenium, Niacin, Vitamin A, and superoxide dismutase. It is understood and herein contemplated that the compositions disclosed herein can be used for treating disease. For example, it is understood that the disclosed compositions can be used to treat cancer or an inflammatory disease. Thus, disclosed herein are methods of treating a disease in a subject comprising administering to the subject a composition comprising a low molecular weight antioxidant and TR4, wherein the antioxidant is selected from the group consisting of Vitamin E, Vitamin C, selenium, Niacin, Vitamin A, and superoxide dismutase. It is understood that one way in which the disclosed vectors and antioxidants can be used in combination to treat the disclosed diseases is to modulate the uptake of the antioxidant. Therefore, disclosed herein are methods of modulating Vitamin E uptake in a subject comprising administering to the subject a vector comprising TR4.

### D. METHODS OF TREATING INFLAMMATORY CONDITIONS AND PREMATURE AGING RELATED DISEASES

Due to the effects of TR4 activity on principal activities associated with aging (e.g., those associated with ROS such as osteoarthritis, rheumatoid arthritis, reactive arthritis, spondylarthritis, systemic vasculitis, juvenile rheumatoid), agents that can increase TR4 activity can be used to treat these conditions. Thus, disclosed are methods for screening agents for comprising administering the agent to a subject and assaying for TR4 activity, wherein an increase in TR4 activity indicates a agent that can be used to treat. It is also understood that administering any of the TR4 vectors and compositions disclosed herein, can be used to treat an inflammatory condition. Thus, disclosed herein are methods of treating an inflammatory condition in a subject comprising administering to the subject a vector comprising TR4. It is understood and herein contemplated that the inflammatory condition can be any condition wherein TR4 activity affects the manifestation of disease. Thus, disclosed herein are methods of treating an inflammatory condition, wherein the inflammatory condition is selected from the group consisting of osteoarthritis, rheumatoid arthritis, reactive arthritis, spondylarthritis, systemic vasculitis, juvenile rheumatoid. It is understood and herein contemplated that one of the ways of treating an inflammatory condition is through the administration of the TR4 vectors and compositions disclosed herein.

The methods and compositions disclosed herein are useful in treating aging and premature aging. One aspect of premature aging involves the Hutchinson-Gilford progeria syndrome (HGPS), commonly referred to as progeria. The landmarks of aging include DNA damage and chromosomal aberrations. Evidence exists for the decline in DNA repair and the accumulation of DNA damage in several different types of cells taken from elderly subjects. Elderly patients' blood and skin cells have less capacity to repair themselves than those from young adults. Furthermore, aging white blood cells with their higher level of DNA damage can explain some of the decline in immune function associated with aging. Disclosed herein are methods of treating a disease related to premature aging in a subject comprising administering to the subject a composition comprising TR4. It is understood that there are many examples of disease that relate to premature aging. Thus, disclosed herein are methods related to treating a disease related to premature aging, wherein the disease is selected from the group consisting of Werner's syndrome, Cockayne Syndrome, Dyskeratosis Congenita, and Hutchinson-Gilford progeria syndrome. The disclosed treatment methods depend on TR4 activity in response to stress, wherein an increase in TR4 activity deceases stress and treats the disease. Thus, disclosed herein are methods of treating a disease related to premature aging in a subject comprising administering to the subject a composition comprising TR4, wherein the TR4 is operably linked to a promoter comprising a stress related element. It is understood that one way to increase TR4 activity is to phosphorylate TR4. Thus, disclosed herein are methods of treating a disease related to premature aging in a subject comprising administering to the subject a composition comprising TR4, wherein the composition further comprises an agent that phosphorylates TR4 at the serine at position 144 (S144). Also disclosed are methods of treating a disease related to premature aging in a subject comprising administering to the subject a composition comprising TR4, wherein the agent dephosphorylates TR4 at the serine at position 351 (S351). It is understood that an agent that phosphorylates TR4 at one residue may also dephosphorylate TR4 at another residue. Thus, disclosed herein are methods of treating a disease related to premature aging in a subject comprising administering to the subject a composition comprising TR4, wherein the agent phosphorylates TR4 at the serine at position 144 (S144), and wherein the agent dephosphorylates TR4 at the serine at position 351 (S351). It is understood and herein contemplated that any of the methods disclosed herein utilizing an agent that phosphorylates S144 and/or dephosphorylates S351 can also be used with a mutant TR4 wherein one or both of the serines at positions 144 and 351 respectively. It is understood that the mutants serines can be mutated to an amino acid that mimics the phosphorylation or dephosphorylation status of TR4. Thus, for example, the mutant can comprise S144D or S351A.

### E. METHODS OF DIAGNOSING A CONDITION

The techniques and methods disclosed herein can be used to assess the likelihood a subject will develop a condition due to decreased TR4 activity. It is understood and herein contemplated that subjects with decreased TR4 activity can have increased DNA damage, including mitochondrial DNA, and any of the other signs or symptoms associated with aging that are known in the art. It is also understood that a subject can be a cell, mammal, mouse, rat, pig, dog, cat, cow, horse, monkey, chimpanzee or other none human primate, or human.

The disclosed methods can also be used to diagnose a condition such as an inflammatory condition, a cancer, or a disease related to premature aging. Thus, disclosed are methods of diagnosing cancer in a subject comprising obtaining a tissue sample from the subject, and measuring the level of TR4 in the sample, such as in the cytoplasm and nucleus of a cell, of the sample, wherein the diagnosis of cancer increases with the increase of TR4 in the cytoplasm. It is understood that the same methods can be used to assess the severity or progression of a cancer. Thus, disclosed herein are methods of assessing the severity of a cancer in a subject comprising obtaining a tissue sample from the subject, and measuring the level of TR4 in the cytoplasm and nucleus of the sample, wherein the severity of the cancer increases with the increase of TR4 in the cytoplasm. Also disclosed are methods of assessing progression of a cancer in a subject comprising obtaining a tissue sample from the subject, and measuring the level of TR4 in the cytoplasm and nucleus of the sample, wherein the severity of the cancer increases with the increase of TR4 in the cytoplasm. It is understood that the methods of assessing cancer progression, assessing the severity of cancer, and diagnosing cancer can involve the comparison of the level of TR4 in the cytoplasm with TR4 levels in other cell compartments such as the nucleus. Thus, for example disclosed herein are are methods of assessing the severity of a cancer in a subject comprising obtaining a tissue sample from the subject, and measuring the level of TR4 in the cytoplasm and nucleus of the sample, wherein the severity of the cancer increases with the increase of TR4 in the cytoplasm relative to the nucleus. It is understood and herein contemplated that the presence of TR4 in a cell can be assayed by any means known in the art. For example, immunohistochemistry using an antibody to TR4 (e.g., the #15 monoclonal antibody disclosed herein). Thus, disclosed herein are methods of diagnosing a cancer, assessing the severity of a cancer, and assessing the progression of a cancer comprising measuring TR4, wherein TR4 is measured by immunohistochemical assay using an anti-TR4 antibody. Specifically disclosed are methods, wherein the tissue sample is blood, muscle, bone, kidney, or liver tissue.

### F. METHODS OF SCREENING FOR AGENTS TO TREAT DISEASE

It is understood herein that the compositions and methods using TR4 disclosed herein can also be used to screen for agents that inhibit DNA damage. Thus, disclosed herein are methods of screening for an agent that inhibits DNA damage comprising administering the agent to a cell and measuring the activity of TR4, wherein a increase in TR4 activity relative to a control indicates an agent that inhibits DNA damage. It is also understood that TR4 activity can increase by modulating the phosphorylation of the protein. Thus, agents that phosphorylate or dephosphorylate TR4 can increase TR4 activity. Thus, for example disclosed herein are methods of screening for an agent that inhibits DNA damage, wherein the agent phosphorylates TR4 at the serine at position 144 (S144). Also disclosed are methods of screening for an agent that inhibits DNA damage, wherein the agent dephosphorylates TR4 at the serine at position 351 (S351). It is understood that an agent that phosphorylates TR4 at one residue may also dephosphorylate TR4 at another residue. Thus, disclosed herein are methods of screening for an agent that inhibits DNA damage, wherein the agent phosphorylates TR4 at the serine at position 144 (S144), and wherein the agent dephosphorylates TR4 at the serine at position 351 (S351). The disclosed screening methods may need a manner to assess TR4 activity in response to damage. Thus, disclosed herein are methods of screening for an agent that inhibits DNA damage, further comprising inducing DNA damage in the cell. It is understood, that the DNA damage can be induced by any method known in the art. For example, the DNA damage can be induced by exposing the cell to UV-irradiation, IR-irradiation, γ-irradiation, or H₂O₂.

The disclosed screening methods can be used with any method for measuring the amount of TR4 expression in a cell. For example, TR4 activity in a cell can be measured relative to a control, wherein an increase in TR4 activity relative to a control indicates an agent that inhibits DNA damage. Because TR4 activity is directly linked to Gadd45 and cockayne syndrome protein B (CSB), disclosed herein are methods of screening for an agent that inhibits DNA damage, wherein TR4 activity is measured by measuring Gadd45a or CSB.

It is understood that the accumulation of DNA damage can cause many diseases, for example, cancer. Thus, the disclosed methods of screening for an agent that inhibits DNA damage can also be used to screen for an agent that inhibits cancer. Therefore, disclosed herein are methods of screening for an agent that inhibits a cancer comprising administering the agent to a cell and measuring the activity of TR4, wherein a increase in TR4 activity relative to a control indicates an agent that inhibits cancer.

### G. COMPOSITIONS

### 1. Molecules that Inhibit TR4 Interactions

a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of TR4 or the genomic DNA of TR4 or they can interact with the polypeptide TR4. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RnaseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of TR4, or fragments thereof, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target molecule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al. Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162

b) Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with TR4 or fragments thereof such that TR4 are inhibited from performing transactivation activity. Antibody also includes, chimeric antibodies and hybrid antibodies, with dual or multiple antigen or epitope specificities, and fragments, such as F(ab′)2, Fab′, Fab and the like, including hybrid fragments, as well as conjugates of antibody fragments and antigen binding proteins (single chain antibodies) as described, for example, in U.S. Pat. No. 4,704,692, the contents of which are hereby incorporated by reference. Antibodies that bind the disclosed regions of TR4 or fragments thereof, such that TR4 decrease their transactivation activity are also disclosed. The antibodies can be tested for their desired activity using the in vitro assays described herein, or by analogous methods, after which their in vivo therapeutic and/or prophylactic activities are tested according to known clinical testing methods. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “monoclonal antibody” as used herein refers to an antibody obtained from a substantially homogeneous population of antibodies, i.e., the individual antibodies within the population are identical except for possible naturally occurring mutations that can be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes can be immunized in vitro, e.g., using the binding domains of the compositions described, herein, such as the ligand binding domain, described herein.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies); Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(2) Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

c) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein, such as TR4 or fragments thereof, can be used as targets for the combinatorial approaches. Also disclosed are the compositions that are identified through combinatorial techniques or screening techniques in which the compositions disclosed in herein, such as TR4 or fragments thereof, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, TR4 fragments thereof, are also disclosed. Thus, the products produced using the combinatorial or screening approaches that involve the disclosed compositions, such as, TR4 or fragments thereof, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods related to combinatorial chemistry)

A preferred method for isolating proteins that have a given function is described by Roberts and Szostak Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA, to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that interactions between synthetic or engineered peptide sequences could be identified which bind a molecule of choice. The benefit of this type of technology is that the selection is done in an intracellular environment. The method utilizes a library of peptide molecules that are attached to an acidic activation domain. A peptide of choice, for example a portion of TR4 is attached to a DNA binding domain of a transcriptional activation protein, such as Gal 4. By performing the two-hybrid technique on this type of system, molecules that bind the portion of TR4 can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to TR4 or fragments thereof for inhibition or activation of TR4 activity is a method of isolating desired compounds.

As used herein combinatorial methods and libraries included traditional screening methods and libraries as well as methods and libraries used in iterative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as TR4, and/or fragments thereof, are also disclosed. Thus, the products produced using the molecular modeling approaches that involve the disclosed compositions, such as TR4 and/or fragments thereof, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling, and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutics Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

d) Methods of Identifying Activators of TR4

Disclosed are methods of identifying an activator of TR4, comprising incubating a library of molecules with TR4 forming a mixture, and identifying the molecules that activate TR4, wherein the activity comprises an upregulation of TR4.

Also disclosed are compositions produced by any of the processes as disclosed herein, as well as compositions capable of being identified by the processes disclosed herein.

Disclosed are methods of manufacturing a composition for enhancing the interaction between TR4 and a ligand thereof, comprising synthesizing the enhancers as disclosed herein.

Also disclosed are methods that include mixing a pharmaceutical carrier with the activator of TR4 as disclosed herein, and produced by any of the disclosed methods.

Disclosed are methods of identifying activators of TR4 comprising, a) administering a composition to a system, wherein the system supports TR4 activity, b) assaying the effect of the composition on the amount of TR4 in the system, and c) selecting a composition which causes a decrease in the amount of TR4 present in the system relative to the system without the addition of the composition.

Also disclosed are methods of identifying activators of TR4 transcription activity comprising, a) administering a composition to a system, wherein the system supports TR4 transcription activity, b) assaying the effect of the composition on the amount of TR4 transcription activity in the system, and c) selecting a composition which causes an increase in the amount of TR4 transcription activity present in the system relative to the system without the addition of the composition.

### 2. Aspects Generally Applicable to Compositions

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example TR4 and/or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety, and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, Ni, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to the genes of TR4, and/or fragments, which can be found at Genbank, at for example, http://www.pubmed.gov and these sequences and others are herein incorporated by reference in their entireties as well as for individual subsequences contained therein.

The disclosed sequences and variants can be founding Genbank. It is understood that the description related to this sequence is applicable to any sequence unless specifically indicated otherwise. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and Probes

Disclosed are compositions including primers and probes, which are capable of interacting with the TR4 nucleic acids as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the TR4 and/or fragments thereof, nucleic acid or region of the TR4 and/or fragments thereof, nucleic acid or they hybridize with the complement of the TR4 and/or fragments thereof nucleic acid or complement of a region of the TR4 and/or fragments thereof nucleic acid.

d) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as nucleic acids encoding TR4 and/or fragments thereof into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is a virus belonging to the virus family of Retroviridae, including any types, subfamilies, genus, or tropisms. Retroviral vectors, in general, are described by Verma, 1M, Retroviral vectors for gene transfer. In Microbiology-1985, American Society for Microbiology, p. 229-32, Washington, (1985), which is incorporated by reference herein. Examples of methods using retroviral vectors for gene therapy are described in U.S. Pat. Nos. 4,868,116 and 4,980,286; PCT applications WO 90/02806 and WO 89/07136; and Mulligan (1993) Science 260:926-32; the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Diol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(c) Adeno-Associated Viral Vectors.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(d) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunol. Rev., 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochim. et Biophys. Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(1) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci._{—}78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

f) Peptides

(1) Protein Variants

As discussed herein there are numerous variants of the TR4 proteins and/or fragments thereof that are known and herein contemplated. In addition, to the known functional TR4 and/or fragments thereof species homologs there are derivatives of the TR4 proteins and/or fragments thereof, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acid | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence can not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

g) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody or other molecule, such as fragment of TR4, for forming or mimicking a TR4/ligand, for example, the efficacy of the therapeutic antibody or fragment can be assessed in various ways well known to the skilled practitioner. For instance, one of ordinary skill in the art will understand that a composition, such as an antibody or fragment, disclosed herein is efficacious in forming or mimicking a TR4 interaction in a subject by observing, for example, that the composition reduces the amount of TR4 activity. The TR4 activity can be measured using assays as disclosed herein. Any change in activity is disclosed, but a 5%, 10%, 15%, 20%, 25%, 30%, 35%, 40%, 45%, 50%, 55%, 60%, 65%, 70%, 75%, 80%, 90%, or a 95% reduction in TR4 activity are also disclosed.

Other molecules that interact with TR4 which do not have a specific pharmaceutical function, but which can be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of TR4 related diseases such as those associated with aging and premature aging.

h) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

i) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

3. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### H. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate including a human, ape, monkey, orangutan, or chimpanzee.

Also disclosed are animals produced by the process of adding to the animal any of the cells disclosed herein.

### I. METHODS OF USING THE COMPOSITIONS AS RESEARCH TOOLS

The compositions can be used for example as targets in combinatorial chemistry protocols or other screening protocols to isolate molecules that possess desired functional properties related to TR4. For example, TR4 and its interaction domains can be used in procedures that will allow the isolation of molecules or small molecules that mimic their binding properties. Libraries of molecules can be screened for interaction with TR4 by incubating the potential binding molecules with TR4 and then isolating those that are specifically active. There are many variations to this general protocol.

The disclosed compositions can also be used diagnostic tools related to diseases such as those associated with aging.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### J. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### TR4 and Genomic Stability

The studies disclosed herein lead to identification of a novel pathway in maintaining the genomic stability, which can extend the understanding of the molecular basis of aging in human and development of strategies to slow this process.

a) DNA Damage and DNA Repair Network:

DNA damage is a common cell death-inducing signal, however, the death program that is activated varies by cell type. DNases and ROS can damage DNA. The mitochondria are a significant source of ROS that are associated with the pathogenesis of many diseases and with aging (Genova, M. L., et al. (2004) Ann N Y Acad Sci, 1011: 86-100; Huang, H. and Manton, K. G. (2004) Front Biosci, 9: 1100-1117). The oxygen species that are typically linked to oxidative stress include superoxide anion, hydroxyl radical (OH⁻), hydrogen peroxide (H₂O₂), nitric oxide (NO) and peroxynitrite (ONOO⁻). Although generation of these species from molecular oxygen is a normal feature of mammalian respiration, ROS directly target DNA resulting in different lesions, such as single- or double-strand DNA breaks (Bohr, V. A., et al (2002). Gene, 286: 127-134). The most frequent oxidative damage to DNA is believed to be the 8-hydroxylation/oxidation of guanine base to 8-hydroxydeoxguanosine (8-OHdG) (Stevnsner, T., et al. (2002) Exp Gerontol, 37: 1189-1196). These lesions disrupt vital processes, such as transcription and replication, which causes growth arrest or cell death. To cope with DNA damage, organisms evolved an intricate network of DNA damage repair pathways, each focusing on a different class of lesion (Lehmann, A. (2002) Curr Biol, 12: R550-551). Premature aging models:

If defects in genome maintenance lead to accelerated aging in human and mice, it is possible that normal aging is caused by inadequately repaired DNA damage. Genotype-phenotype correlations in mouse models of defects in genome maintenance can provide valuable insights into the basic mechanisms of aging and natural defense systems that promote longevity. In addition to mice, several human diseases exhibit symptoms of acceleration of aging. No disease condition displays all symptoms of accelerated aging. Diseases that resemble certain aspects of accelerated aging are known as segmental progerias, because of segments of aging in each disease condition. Segmental progerias include disease of DNA-damage/repair (such as Werner's syndrome (Bohr, V. A., et al. (2002) Biogerontology, 3: 89-94) and xeroderma pigmentosum), and diseases showing telomere abnormalities (such as Hutchinson-Gilford syndrome and Down's syndrome) (Brown, W. T. (1987) Curr Probl Dermatol, 17: 152-165; Martin, G. M. (1982) Natl Cancer Inst Monogr, 60: 241-247; Brown, W. T. (1990) Annu Rev Gerontol Geriatr, 10: 23-42).

b) TR4 Orphan Receptor and Generation of TR4 Knockout Mice:

The nuclear orphan receptor testicular receptor 4 (TR4), was first identified by cloning from prostate and testis cDNA libraries (Chang, C., et al. (1994) Proc Natl Acad Sci USA, 91: 6040-6044). In vitro data suggest that TR4 functions as a master regulator to modulate many signaling pathways, including maintenance of erythrocyte progenitor populations, in the case of the human erythropoietin gene (EPO) (Kim E, et al. 2003 J Biol Chem 278:46919-26), through roles in the process of neurogenesis, in the case of the ciliary neurotrophic factor alpha (CNTFRα) Young W J, et al. 1997 J Biol Chem 272:3109-16; Young W J, et al. 1998 J Biol Chem 273:20877-85), interfering with retinoic acid/RAR/RXR (Lee Y F, et al. 1998 J Biol Chem 273:13437-43), T3/T3R (Lee Y F, et al. 1999 J Biol Chem 274:16198-205), AR-mediated pathways (Lee Y F, et al. 1999 Proc Natl Acad Sci USA 96:14724-9), and facilitation of viral infection and propagation, in the case of HPV-16 and SV40 (Lee H J, et al. 1995 J Biol Chem 270:30129-33). In addition, the physiological functions of these receptors was explored based on information collected from previous studies of tissue distribution and target gene regulation. Furthermore, as TR4 remains an orphan, a greater understanding of their physiological roles may provide significant clues as to the natural ligands by which they may be activated. Therefore, mice lacking TR4 (TR4 KO) were generated via targeted gene disruption (Collins, L. L., et al. (2004) Proc Natl Acad Sci USA, 101: 15058-15063). The lambda KOS system was used to derive a TR4 targeting vector. Three independent genomic clones spanning exons 4-10 were isolated. The targeting vector was derived from one clone and contained a 2173 bp deletion that included most of exon 4 and all of exon 5. The genomic sequence encoding the DBD of TR4 was replaced by a Lac-Z/Neo selection cassette. The Not I linearized vector was electroporated into strain 129SvEv^{brd} (LEX1) embryonic stem (ES) cells, and G418/FIAU-resistant ES cell clones were isolated and screened by Southern blot for homologous recombination of the mutant DNA. One targeted ES cell clone was injected into blastocysts of strain C57BL/6 (albino), which were then inserted into pseudopregnant female mice for continuation of fetal development. Resulting chimeric male mice were then mated to C57BL/6 (albino) females to generate animals heterozygous for the mutation. The TR4 KO mice demonstrate high rates of early postnatal mortality, as well as significant growth retardation. TR4 KO mice also display reproductive defects, in which reduced fertility was seen in both genders (Collins, L. L., et al. (2004) Proc Natl Acad Sci USA, 101: 15058-15063), abnormalities in spermatogenesis (Mu X, et al. 2004 Mol Cell Biol 24:5887-99), and cerebella development (Chen Y T, et al. 2005 Mol Cell Biol 25:2722-32).

The surviving adult TR4 KO mice develop growth impairments, including growth retardation, hypoglycemia, and mild late-onset myopathy where mitochondria-like proliferation inclusions were found. Decline of mitochondria function is often linked to aging related syndrome (Stevnsner, T., et al. (2002) Exp Gerontol, 37: 1189-1196; Roubertoux, P. L., et al. (2003) Nat Genet, 35: 65-69). By 6 months, most of the mice develop kyphosis and a sign of osteoporosis with a reduced bone mineral density (BMD). A premature ovarian failure was observed in three 6-month-old TR4 KO females, in which there was no active estrus cycle and complete anovulation. All these phenotypes indicate TR4 KO mice develop premature aging. TR4 KO mice embryonic fibroblast (MEF) cells display a dramatic reduction in replicative lifespan. Emerging late age-onset phenotypes observed in TR4 KO mice include abnormal mitochondria proliferation and reduction of MEF replicative lifespan. This indicates that TR4 plays an important role in maintaining the genome stability and loss of TR4 in mice leads to development of systemic problems that cause the premature aging process.

c) Results

(1) Shorter Lifespan and Premature Aging in TR4 KO Mice:

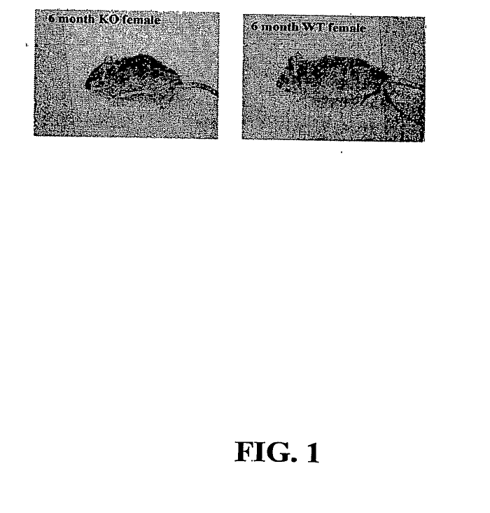

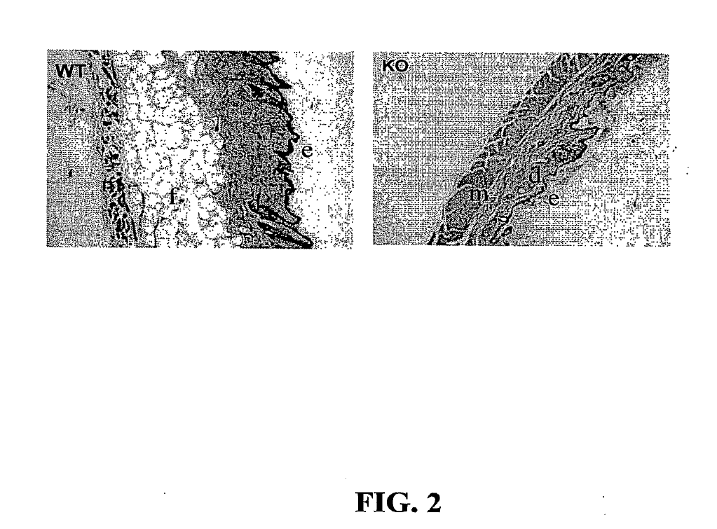

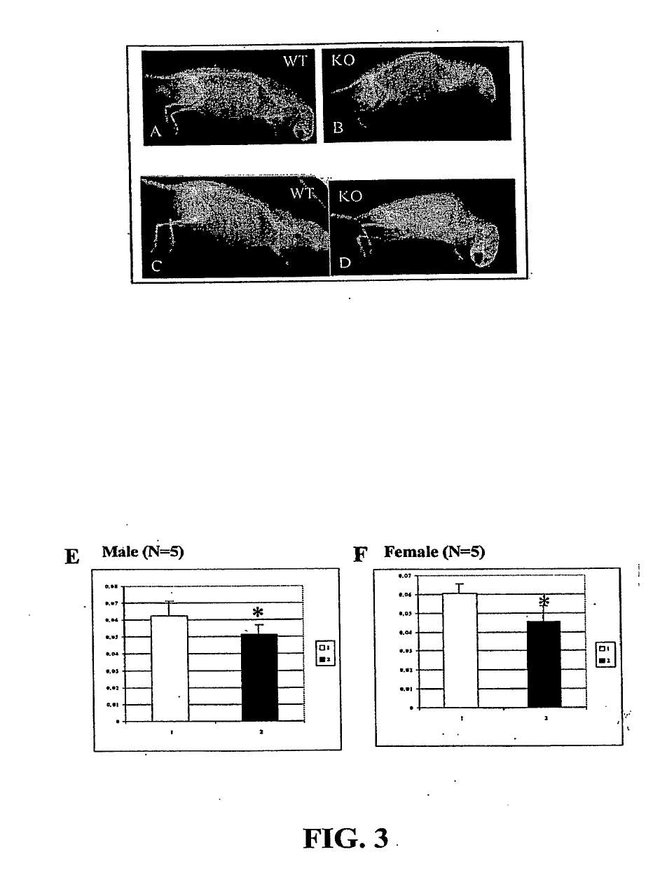

TR4 KO mice, in general, have shorter lifespan (average <12 month, compared with >2 years from wt mice). More than 95% of the TR4KO mice (67 out of 70 TR4 KO mice) die within one year of age, without an obvious cause of death. Adult TR4 KO mice display growth impairments, including reduction of body weight and hypoglycemia. By 6 months, the TR4 KO mice acquired “aged” appearances in which some mice have greasy hair (FIG. 1), significant reduction of dermal thickness and absence of subcutaneous adipose cells (FIG. 2), and severe kyphosis (curvature of the spinal column) significant global bone mineral density reduction (FIG. 3). All of which indicate that TR4 KO mice develop a premature aging process.

(2) Rapid Replicative Senescence in TR4KO MEFs Contributes to Higher Cellular ROS Levels:

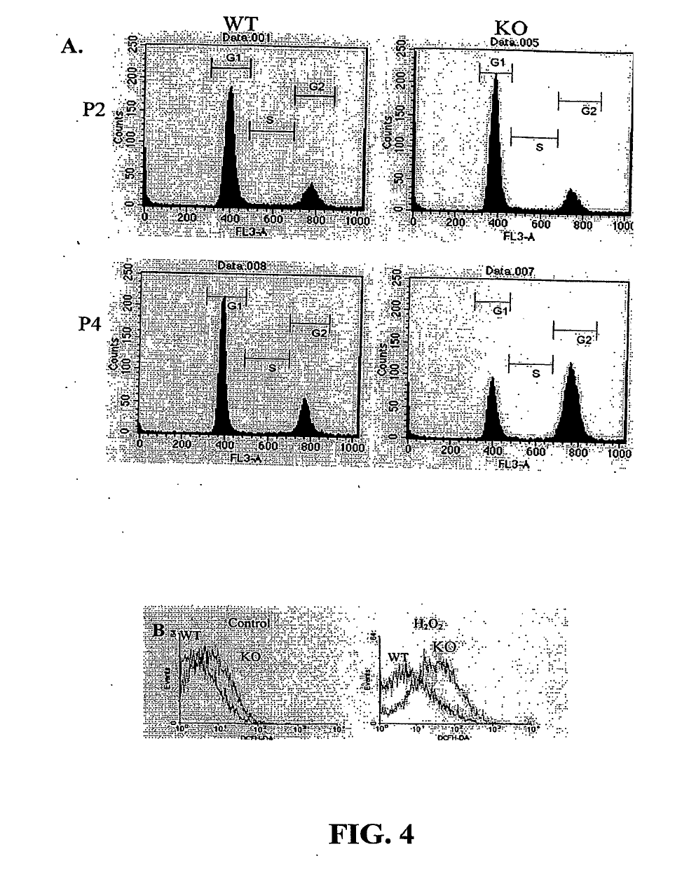

Replicative senescence has been employed as a cellular model for aging and it occurs primarily in response to oxidative DNA damage (Campisi, J. et al. (2005) Cell, 120: 513-522). Wt MEFs grow well for approximately 2-3 doublings (P2-3) before decreasing in proliferation, and after 8-10 doublings, the cell senescence occurs. In contrast, TR4 KO MEFs proliferation drastically decline after P4 (FIG. 4A). ROS contribute to cells senescence, and how cells respond to oxidative stress would determine the life span. The amounts of cellular ROS in MEFs were determined by flow cytometry using DCFH-DA. As shown in FIG. 4B, cellular ROS level, shown as fluorescence intensity, is higher in TR4 KO than in wt MEFs. When MEFs were exposed to H₂O₂ and measured cellular ROS levels, the amounts of fluorescence in both in wt and TR4 KO increased after H₂O₂ treatment; TR4 KO MEFs accumulated more ROS than wt MEFs.

(3) Increased DNA Single-Strand Breaks in TR4 KO MEFs:

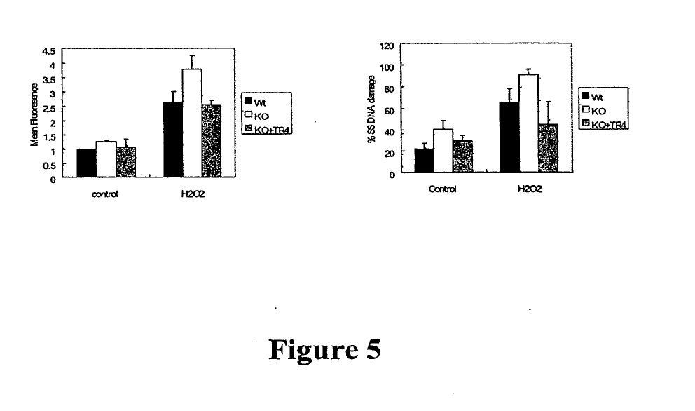

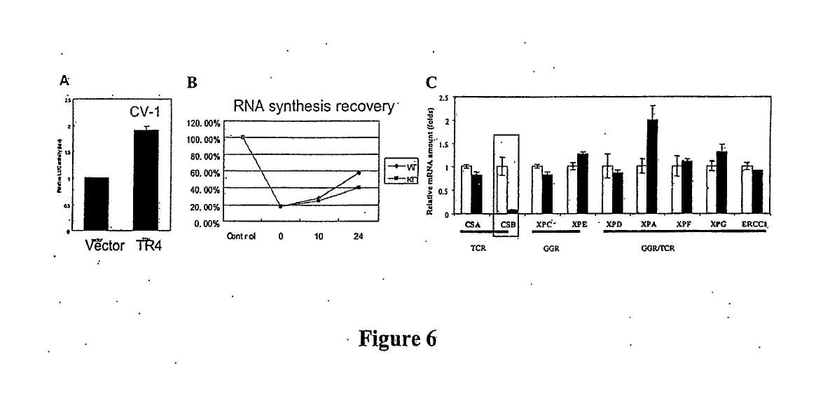

Higher ROS consequently result in more DNA damage and lead to early onset of cell senescence. This was confirmed by measuring the single-strand (SS) DNA breaks in MEFs before and after H₂O₂ treatment. As shown in FIG. 5, the intrinsic, as well as extrinsic (H₂O₂-induced), SS DNA breaks were found increased in TR4 KO MEFs than in wt MEFs and restoring TR4 in TR4 KO reduced SS DNA breaks and restoring TR4 can reduce cellular ROS levels and DNA damages.

(4) TR4 Induces TCR:

A DNA repair assay was used to monitor in vivo reactivation of a promoter activity upon repair of UV-damaged DNA in the reporter plasmid. Overexpression of TR4 in CV-1 cells showed a better reactivation of SV40 promoter activity than with the UV-damaged reporter plasmid and an empty vector. RNA synthesis recovery rate, a sign of TCR efficiency was reduced in TR4^{-/-} cells. After screening the genes involved in the NER pathway, only CS-B showed significant decrease in TR4^{-/-} MEF cells (white bar).

(5) Up-Regulation of TR4 Expression Under Genotoxic Stress:

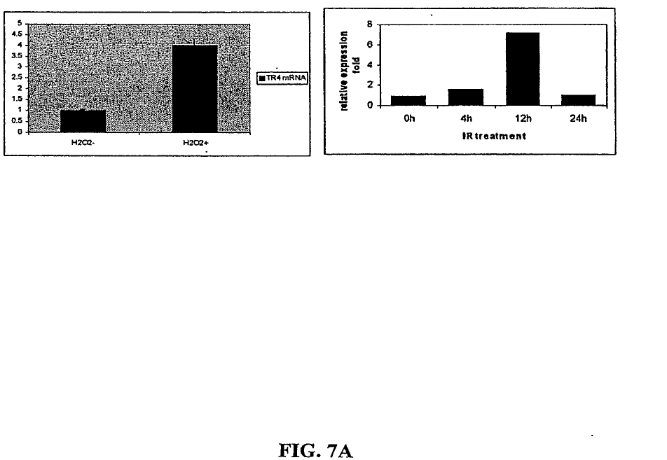

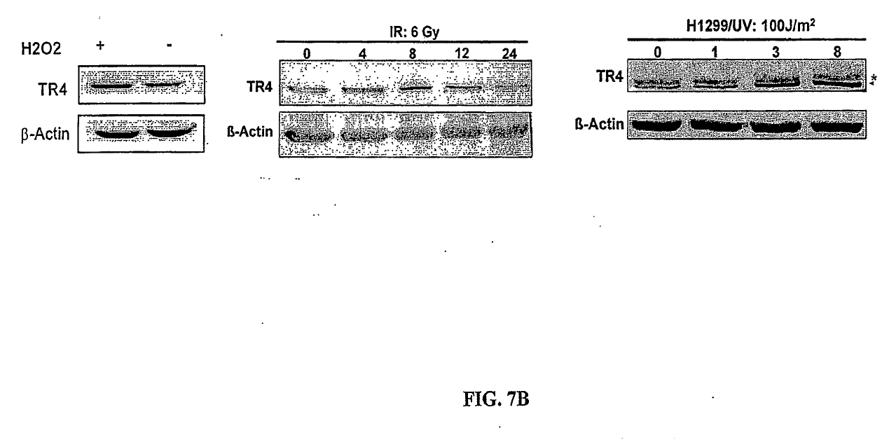

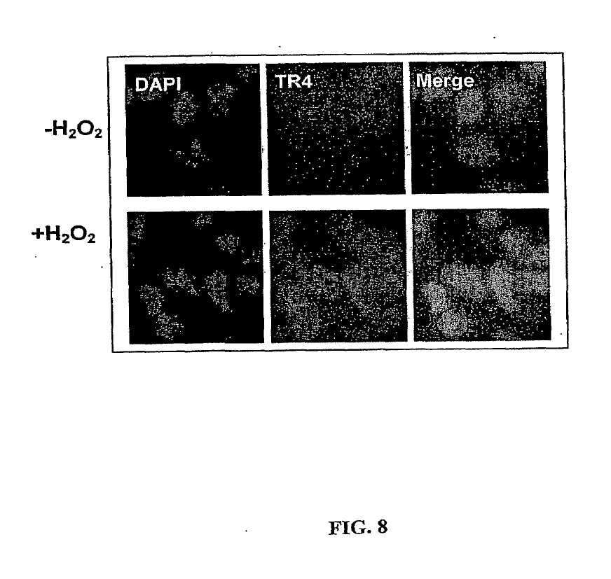

TR4 expression levels were examined upon the genotoxic stress, H₂O₂, UV, and IR by Q-PCR (FIG. 7A) and Western Blotting (FIG. 7B). As shown in FIG. 7A, TR4 mRNA expression was increased 2 hrs after H₂O₂ challenge. The same tendencies were also seen in IR-treatment. All these findings indicate that TR4 is involved, not exclusively, in a subset of checkpoint proteins that monitor cell-cycle progression, such as sensor proteins, or in translating these DNA-derived stimuli to biochemical signals and then to modulate the functions of specific down-stream target proteins. As shown in FIG. 7B, TR4 protein expression increased 12 hrs after H₂O₂ challenge. The same tendencies were also seen in IR and UV treatments. In addition, the subcellular localization of TR4 after H₂O₂ treatment was examined. As shown in FIG. 8, TR4 shifted from the cytoplasm to the nucleus after 2 h exposure to H₂O₂. All these findings indicate that TR4, as a transcriptional factor, responds to stress that can either translate these DNA-derived stimuli to biochemical signals and/or modulate specific down-stream target proteins and that nuclear TR4 is required for such actions.

(6) TR4 Regulates Gadd45a Gene Expression.

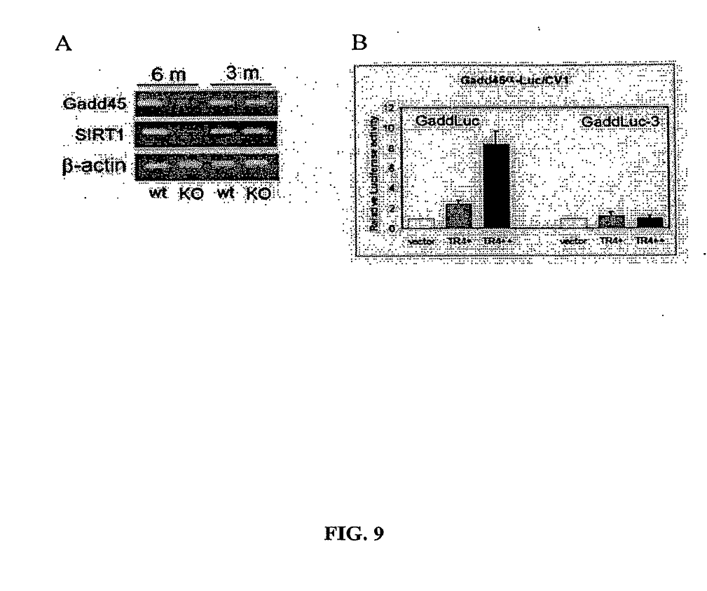

In searching for potential TR4 direct targets, Gadd45a, a growth arrest and DNA damage response gene appears to be a direct target of TR4 that is able to mediate part of TR4 effects on DNA repair. As shown in FIG. 9A, a reduction of Gadd45a mRNA was seen in TR4 KO mice muscle from both 3- and 6-month old mice as compared to their wt littermates. To test if this was via transcriptional regulation, a Gadd45a promoter reporter gene (GaddLuc), containing putative TR4 responsive element, was co-transfected with pCMX-TR4 or vector control, and examined for Luc activity. As shown in FIG. 9B, TR4 activated GaddLuc activity in a TR4-dose dependent manner, but not the GaddLuc without DR3-motif.

(7) TR4 KO Fibroblasts are More Sensitive to Oxidative Stress Mediated by H₂O₂.

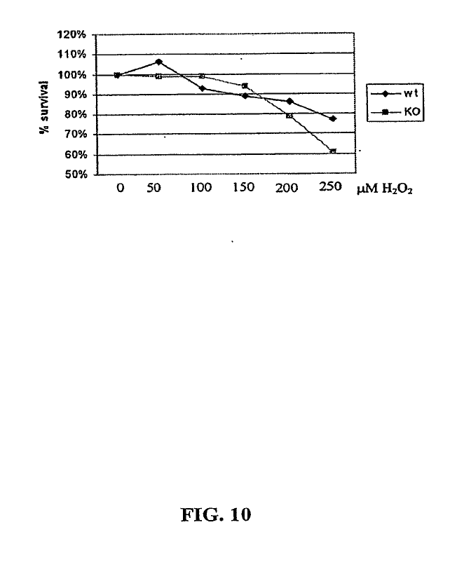

A reduced ability to tolerate stress is a hallmark of aging. How TR4 and TR4 KO MEFs respond to ROS(H₂O₂) was examined. As shown in FIG. 10, TR4 KO MEFs are more sensitive to H₂O₂, and fewer TR4 KO MEFs survive compared with wt MEFs.

(8) TR4 Induces Repair of UV-Induced DNA Damage.

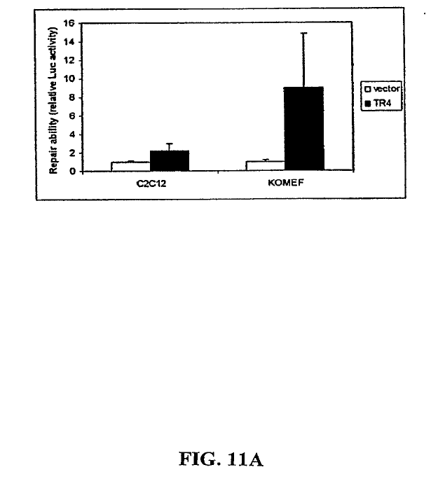

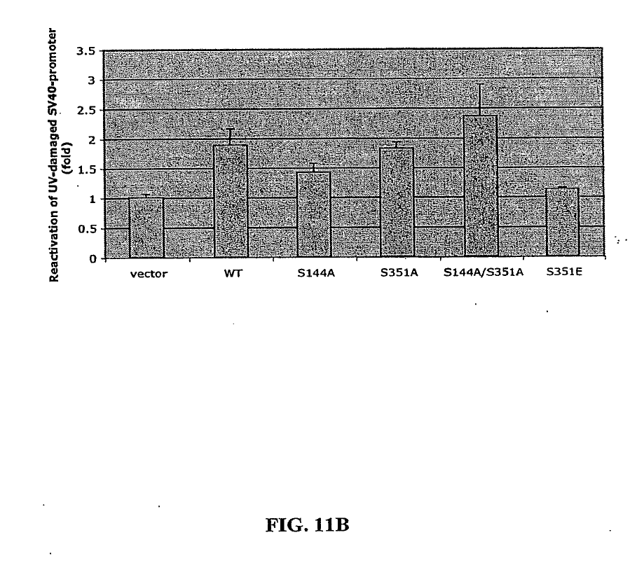

To determine if TR4 mediates UV-damaged DNA repair, a Luciferase-based DNA repair assay was used which measures transcriptional couple repair (TCR) ability (Tran H, et al. 2002 Science 296:530-4). The principle of this assay is to monitor reactivation of a promoter activity upon repair of UV-damaged DNA in the reporter plasmid in vivo. As shown in FIG. 11A, CV-1 cells transfected with the UV-damaged reporter plasmid with pCMXTR4 show a better reactivation of SV40 promoter activity than with empty vector. More importantly, restoring TR4 in TR4 KO MEF cells also confirmed that TR4 regulates UV-damaged DNA repair vector (FIG. 11A). To verify that DNA repair mediated by TR4 is not due to a particular cell type, the same assay was performed in mouse C2C12 cells. As shown in FIG. 11B, C2C12 cells transfected with the UV-damaged reporter plasmid with pCMXTR4 show a better reactivation of SV40 promoter activity than with empty vector (FIG. 11B). To test whether phosphorylation of TR4 affects TR4 repair ability, putative phosphorylation sites were searched for using the scansite computer program at MIT. It was found that Serine at 351 (Ser-351) is a highly stringent binding site for 14-3-3 and is conserved from mouse to human Ser-351, and Ser-144 is a phosphorylation site for PKCα, β, γ, and ξ involving in NER pathways. Additionally, it was found that the S351A mutant induced DNA repair effectively and conversely, the DNA repair is abolished with the S351E mutant, and expression of S144A reduces DNA repair efficiency as compared to wt TR4. These results strongly indicate that dephosphorylation of TR4 at Ser-351, and phosphorylation of Ser-144 are essential for inducing UV-damaged DNA repair. Interestingly, bacterial (non-transcription-active) plasmid, pBluescript, was not repaired more efficiently in CV-1 overexpressing TR4, indicating that TR4 is not involved in global genomic repair (GGR). Thus, TR4 mediates transcription coupled repair (TCR) induced by UV irradiation in both human and mouse cells.

(9) Loss of vitamin E-mediated anti-ROS effects in TR4 KO fibroblast.

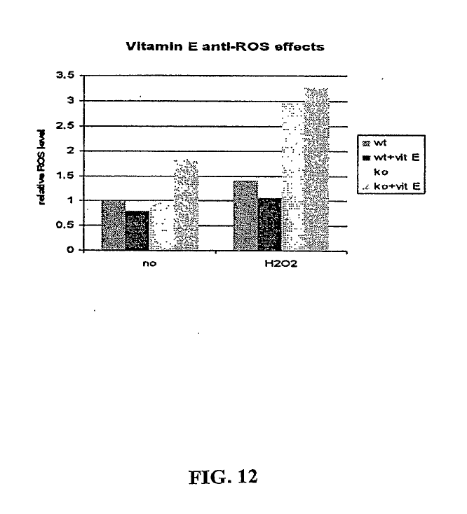

To test if TR4 is involved in low molecular weight antioxidant (LMWA), such as vitamin E anti-oxidant effects, fibroblasts from TR4 KO and wt were challenged with 250 μM H₂O₂ in the presence of 100 nM α-tocopherol and VES. As shown in FIG. 12, TR4 KO fibroblast lost the vitamin E-mediated anti-oxidant effect while a reduction of ROS was seen in wt.

(10) The Structure and Functional Study of TR45′-Promoter:

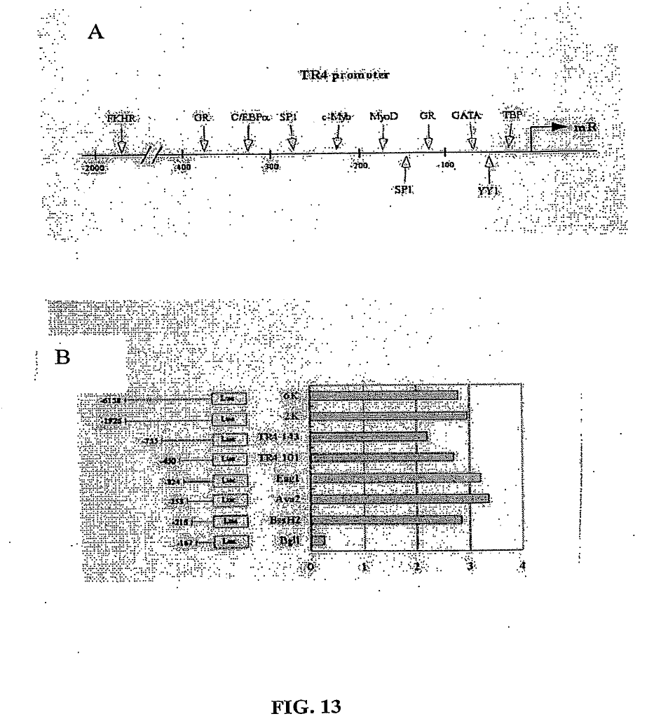

To investigate how stress influences TR4 expression at transcriptional level, a 6.0 kb genomic DNA fragment containing the TR4 gene promoter region was cloned, sequenced, and characterized. Sequence homology search within this promoter region revealed potential cis-acting elements which can be recognized by several transcriptional factors such as GR, C/EBPα, SP1, YY1, and MyoD. Deletion analyses and Luciferase assay showed a potential enhancer element, within 216 to 167 bp upstream of the transcription start site (FIG. 13), which is associated with the TR4 transcriptional activity.

(11) Construction of TR4 RNAi:

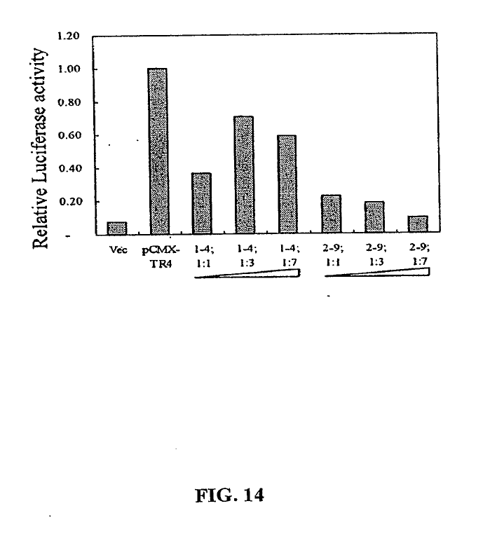

As shown in FIG. 14, two clones of TR4 RNAi (1-4, and 2-9) were constructed into pSuperior.retro.puro (OligoEngine) vector, and their ability to suppress TR4-mediated TR4RE-Luc activity was tested. Clone 2-9 TR4RNAi showed a better suppression effect, and is used in these studies.

In summary, the data showed that lack of TR4 in mice results in accelerated aging, and rapid cellular senescence that are contributed to un-repaired DNA damage caused by excess amounts of oxidative free radical insults. And, TR4 is a stress responsive protein in which TR4 can be induced by ROS and IR. All these data strongly indicated that TR4 is critical in maintaining the genomic stability.

(12) Investigation of the Roles of TR4 in Surveillance of the Genomic Toxicity.

The genomes of eukaryotic cells are under continuous insults from the environment and intracellular metabolism byproducts. To ensure cells pass accurate genome information to the next generation, cells develop a series of surveillance pathways to detect damaged DNA and abnormal DNA structures as well as to coordinate cell-cycle progression with DNA repair. The expression of TR4, a transcriptional factor, is induced by oxidative stress, and the activation of TR4 suppresses cellular ROS, and reduces DNA damage; therefore the inactivation of TR4 results in genomic instability and leads to premature aging. Thus, TR4 is a stress responsive molecule that can promote cellular defense signals to protect cells from DNA-damage. To investigate TR4 roles in these cellular surveillance-defense systems, the alterations of TR4 expression in response to a variety of DNA-damage stress are measured, and are correlated these changes to TR4 transactivation activity and to TR4-mediated biochemical signal transduction pathways in cell defense systems. Moreover, the molecular mechanisms underlying how stresses induce TR4 activity is determined by identification of the factors that can modulate the TR4 activity under the stress challenge.

(13) Determination of the Roles of TR4 in Response to a Variety of DNA damage Stresses-Oxidative Stress, IR and UV.

Diverse factors that include DNA repair systems, cell cycle regulation systems, antioxidant defense systems, stress-responsive proteins, and some intracellular communication systems are involved in response to stress. To understand more about TR4 functions in response to DNA damage stress, whether a variety of DNA damage-induced genotoxic stresses such as UV, IR, and H₂O₂ can activate TR4, and whether TR4 activation can reduce cellular DNA damage is examined. Also cell survival between TR4 KO and wt MEFs can be compared in the context of stress sensitivity.

(a) Examination of TR4 Expression Alterations in mRNA and Protein in Response to UV, IR, and H₂O₂ in Wt MEFs.

Determination of how environmental assaults induce TR4 expression contribute to the understanding of TR4 roles in the cellular defense system. In comparison with H₂O₂, it can be determined that IR and UV can stimulate TR4 expression, and the time needed for different stress to induce TR4. MEFs from wt mice are treated with three types of DNA-damage stress, H₂O₂ (50, 100, 200, 500 μM, to 1 mM), UV (2, 4, 8, 16, and 32 J/m2), and irradiation (3, 6, 9, 12, and 15 Gy) ranging from low to high doses and then the treated cells are harvested at different times, from short time (1 h, 2 h, 4 h, and 8 h) to 1 day, 2 days, and 3 days to examine TR4 expression. Total RNA and protein are prepared and analyzed by Q-PCR and Western blotting analysis. The TR4 expression patterns can be confirmed in response to diverse stress in other cell lines, such as H1299 (high endogenous TR4 without functional p53), CHO (medium TR4 expression), and C2C12 (low endogenous TR4).

(b) Comparison of the Stress Sensitivity Between TR4 KO and wt MEFs.

Organisms generally undergo qualitative changes with aging and their biological functions, especially the ability to tolerate stress, gradually degenerate and become more susceptible to stress. To acquire resistance, cells have to induce antioxidant defense and DNA-repair systems. As shown herein, TR4 KO MEFs are more sensitive to H₂O₂ challenges, which indicate a role of TR4 in protecting cells against oxidative stress. It is important to know if TR4, in general, is able to defend cells against different DNA damage insults. Cells that contain TR4 (wt MEFs) are more resistant to stress than cells without TR4 (TR4KO MEFs). Thus, cellular sensitivity to IR and UV is compared between TR4 KO and wt MEFs. Sensitivity to IR and UV is determined by exposure of cells to IR (0, 3, 6, 9, 12, and 15 Gys) and to UV (0, 1, 2, 4, 8, 16 J/m2) and harvesting the cells at day 1, 3, and 5. Cell survival rate is determined by cell growth and proliferation rate using ³H-tymidine incorporation and MTT assays.

(c) Comparison of Degree of DNA Damage Induced by Genotoxic Stress in TR4 KO and Wt MEFs.

Genotoxic insults induce DNA damage and consequently result in altering the cell cycle and activation of DNA repair. Overproduction of ROS and/or defects in DNA repair ability results in unrepaired DNA damage leading to cellular disfunctions, such as early onset of aging in TR4 KO. Thus, the DNA damage between TR4 KO and wt cells is compared when exposed to ROS, IR, and UV. MEFs from wt and TR4 KO are treated with 250 μM H₂O₂, 8 Gy IR, and 10 μm2 UV and cells are harvested post-treatment (1 h, 2 h, 4 h, and 8 h to 1 d, 2 d, and 3 d). DNA damage is determined by DNA precipitation and comet assays. To further confirm anti-DNA damage action of TR4, TR4 is restored to TR4 KO MEFs by viral TR4 gene transfer deliver system to see if restoring TR4 activates the anti-DNA damage defense systems.

(14) Determine if TR4 Mediated anti-DNA Damage is Gadd45a Dependent.

Herein it was found that TR4 can protect cells from the oxidative DNA damage-induced cellular decay, possibly via the modulation of Gadd45a gene activity. Therefore, restoring Gadd45a into TR4 KO cells would rescue some of the TR4-mediated cellular protective effects, and blocking of Gadd45a in wt MEFs would result in loss of TR4 protecting effects. Here, stress activates TR4 to transinduce its targeted gene, Gadd45a, for cellular defense is confirmed. MEFs from TR4 KO, wt, and TR4 KO transfected-TR4 are used to examine Gadd45a expression (endogenous, and stress-response), as well as to examine how Gadd45a-transfected TR4 KO cells and Gadd45a RNAi-transfected wt cells respond to stress.

(a) Determination if Up-Regulation of TR4 can Stimulate TR4 Target Gene, Gadd45, Activity.

It is very important to determine if the up-regulation of TR4 expression can be translated into the activation of TR4. Therefore, TR4 activation by DNA damage inducers stimulates TR4 target gene Gadd45, a growth arrest and DNA damage response gene. First, Gadd45aLuc which contains DR3TR4REs is transfected into MEFs (wt vs TR4 KO) and then cells are exposed to H₂O₂, IR, and UV. To further confirm that DNA-damage-induced Gadd45-Luc activity is mediated through TR4, knockdown of TR4 by TR4 RNAi in wt MEFs, and restoration of TR4 in TR4 KO MEFs are applied to examine the Gadd45-Luc activity.

Next, the expression of endogenous Gadd45a mRNA/protein levels upon the DNA-damage insults between MEFs of TR4 KO and wt are compared by Q-PCR and Western blotting analysis. To confirm a direct regulation of TR4 on Gadd45a gene, Gadd45a changes are examined by applying TR4 RNAi knockdown in wt and retroviral-TR4 rescue in TR4 KO. Finally, EMSA, DNA pull-down, and ChIP assays are performed to illustrate a direct in vitro binding of the putative DR3-TR4RE in Gadd45a promoter with TR4 proteins.

(b) Confirmation of TR4-Mediated Anti-DNA Damage System Via the Modulation of Gadd45a.

As shown herein, TR4 regulates Gadd45a-5′-promoter containing Luc reporter gene activities in a TR4-dose dependent manner in transient transfection assays. TR4 can protect cells from DNA-damage through, at least, partial mediated up-regulation of Gadd45a. To further confirm this, blocking endogenous Gadd45a by RNAi is used to test the loss of TR4 protecting effects in cells. MEFs from wt cells are stably transfected with Gadd45a RNAi (p-super vector) and scramble RNAi control and then tested their response to genotoxic challenge. Meanwhile, the determination is made that decreased DNA damage protective effects in TR4 KO is restored by restoring Gadd45a in TR4 KO cells.

(15) Investigate the Molecular Mechanisms Underlying How Stress Modulates TR4 Activity.

TR4 is a stress responsive protein in which TR4 mRNA levels were increased after H₂O₂ and IR treatment. How stress induces TR4 expression is important. The molecular mechanisms underlying how stress activates TR4 are investigated. The findings show that stress can induce TR4 response in two ways, through transcriptional regulation and post-translational modification.

(a) Via Transcriptional Regulation:

The modulation of TR4 activity can be achieved via regulation of TR4 expression levels, therefore study of the 5′-TR4 reveals how genotoxic stress influences TR4 activity. Based on the findings, TR4 mRNA was up-regulated under H₂O₂ and IR stimulation, which indicated that the TR4 promoter contains a stress-responsive element (SRE) corresponding to the stress. A 6 kb TR45′-flanking region and its serial deletions have been cloned and constructed into Luciferase reporter genes. The transcriptional activity is tested on the 5′-TR4-Luc and its serial deletions upon challenges such as DNA damage agents (oxidative stress (H₂O₂-treatment), UV, and IR) to determine the SRE.

(b) Via Post-Translational Modification:

To sense DNA-damage, cellular signal transduction kinase cascade coordinates cell defense events to maintain genome integrity. To respond to DNA damage, TR4 is a phosphorylated protein in which its activity can be modulated by kinase/phosphotase cascades involved in cell DNA-damage repair systems. Using the motif screening on TR4 molecule (www.scansite.mit.edu), several conserved phosphorylation sites have been found, for instance, Serine (Ser)-351, a highly stringent binding site for 14-3-3, and Ser-144, a highly stringent phosphorylation site for PKCα, β, γ, ξ. To test whether the phosphorylation status affects TR4 activity, Ser is mutated to Alaine to mimic TR4 dephosphorylated form and Ser to Glutamic acid to mimic a phosphorylated form of TR4. TR4 activity under these modulation is measured. Next, the specific kinases and/or phosphatases are tested for involvement in stress-mediated TR4 activation, with CoIP of TR4 kinase and phosphatase performed to test their interaction.

(c) Detailed Methods:

(i) Determination of TR4 Expression by Real-Time PCR (Q-PCR) and Western Blotting Analyses.

MEFs from wt mice will be treated with different doses of H₂O₂, UV, and IR, and harvested at different time according to the designs described in herein. The RNA samples are obtained by Trizol reagents, and total RNA re converted into first strand cDNA by SuperScript III reverse transcriptase (Invitrogen). Primers for amplification of TR4 are designed by the Becon Primer Designs software. Q-PCR are performed using Bio-Rad iQ cycler. CT values are calculated and normalized to the level of the housekeeping gene a-microglobulin. Relative gene expression is calculated according to 2-ΔΔCT from three independent experiments. To confirm the expression changes in protein level, cells are lysed by RIPA buffer and quantified. Proteins are separated by 12% SDS-PAGE and blotted with anti-TR4 antibody (#15 monoclonal antibody) to detect changes in TR4 expression level upon stress. In addition to MEFs, TR4 expression level in response to DNA-damage inducer is measured in H1299 cells (expresses high levels of TR4) and CV-1, and C2C12 (expresses less amount of TR4).

(ii) Cell Proliferation Assay:

Cell proliferation rate is determined by 3H-tymidine incorporation analysis and MTT assays. The response to stress between the TR4 KO MEFs and wt MEFs is compared as shown in percentage of cell survival upon low to high doses of stress treatment. Stress-treated surviving cells are calculated as the ratio of cell number in treated group to non-treated group. For ³H-tymidine incorporation analysis, cells will be incubated for 24 h with medium containing 0.25 μCi/ml 3H-thymidine. The radioactivity incorporated is measured by liquid scintillation counting. For MTT assay, the conversion of a colorless substrate to reduced tetrazolium by the mitochondrial dehydrogenase, is used to assess cell viability and growth. After each treatment period, 10% volume of medium of thiazolyl blue (5 mg/ml, Sigma) is added into each well for 2-3 h at 37° C. The resultant precipitate is dissolved in 0.04 M HCl in isopropanol and absorbency is read at a wavelength of 570 nm with background wavelength at 660 nm.

(iii) DNA Single-Strand Breaks:

A DNA precipitation assay is used for DNA-strand-breaks detection. Confluent MEF cells are labeled with 0.25 μCi/ml [³H]methylthymidine for 24 h. Cells are treated with various DNA-damage inducers. After treatment, the cells are washed with PBS and lysed with lysis buffer (10 mM Tris/HCl/10 mM EDTA/50 mM NaOH/2% SDS) followed by addition of 0.12 M KCl. The lysate is incubated for 10 min at 65° C. followed by a 5 min cooling-and-precipitation period on ice. A DNA-protein K-SDS precipitate is formed under these conditions, from which low-molecular-mass broken DNA is released. This DNA are recovered in the supernatant from a 10 min centrifugation at 200 g, 10° C., and transferred into a liquid scintillation vial containing 1 ml of 50 mM HCl. The precipitated pellet (intact double-stranded DNA) is solubilized in 1 ml of water at 65° C., the tube rinsed with 1 ml of water, and 8 ml of scintillation fluid added to each vial. The amount of double-stranded DNA remaining is calculated for each sample by dividing the d.p.m. value of the pellet by the total d.p.m. value of the pellet+supernatant and multiplying by 100. The results representing the extent of DNA damage are calculated as (Dt/Dc)×100, where Dt represents double-stranded DNA in treated cells and Dc represents double-stranded DNA in the respective control cells. In control cells (cells incubated in Ca²⁺-containing or Ca²⁺-free/EGTA), the level of total double-stranded DNA is around 75%. Pretreatment with various chelators did not affect this level (Jornot, L., et al. (1998) Biochem J, 335 (Pt 1): 85-94).

(iv) Comet Assay:

An Fpg-FLARE (fragment length analysis using repair enzymes) comet assay kit are used in accordance with the manufacturer's instructions (Trevign, Ginthersberg, Mo.). This kit specifically detects oxidative DNA lesions such as 8-oxo-2-deoxyguanosine and formamidopyrimidines. Images of 50 randomly chosen nuclei per sample are captured using a CCD camera coupled to an epifluorescence microscope. Comet tail lengths are measured using the comet macro from NIH public domain image analysis program.

(v) Transfection Assay and Luciferase Assays.

The 6 kb and serial deleted constructs with Luc reporter is transfected into CV1 cells, and then cells are treated with H₂O₂ (250 μM), UV, and IR. The region(s) that lose the response to H₂O₂-induced 5′-TR4-Luc activity are potential SREs. More stress challenges, such as UV-irradiation, ionizing radiation, and low glucose are applied to determine the SREs within the TR45′-promoter. The putative SRE regions that are critical for stress response are further narrowed down by site-directed mutagenesis. The goal is to identify the minimal regions, around 30-50 bp, responsible for the stress-induced TR4. Transient transfection is performed by using SuperFect according to the manufacturer's suggested procedure (Qiagen). After transfection, cells are treated with 250 μM H₂O₂ for 2 hs, and then medium is replaced with fresh culture medium for 48 h. Cell lysates are prepared and the luciferase activity is normalized for transfection efficiency using pRL-CMV as an internal control. Luciferase assays are performed using dual-luciferase reporter system (Promega).

(vi) Site-Directed Mutagenesis of Potential SRE in TR4 Promoter.

If putative SREs identified from the serial deletion TR45′-promoter study, contain some known cis-acting elements, the sequences in these cis-acting elements are mutated by using QuickChange Site-Directed mutagenesis kit (Strategene). When regions are identified that contain no known cis-acting elements, the regions are mutated every 15-20 bp to narrow down the minimal regions for TR4 activation.

(vii) Construction of Gadd45 RNAi.

To generate Gadd45 RANi, the system is applied through OligoEngine (www.oligoengine.com) for specialized design software. The pSUPER vector is used to express the small RNA molecules to achieve long-term silencing of endogenous Gadd45a. Synthetic DNA oligos encoding two 19-nt reverse complements homologous to a portion of Gadd45a, separated by a short spacer region, is inserted into the vector. When expressed under the control of the polymerase-III based expression system, the RNA transcript will form a short hairpin structure with a 19 base-pair double-stranded region and two final uridines overhanging the 3′ end to generate siRNA for Gadd45 knockdown. After sequence confirmation, Gadd45 RANi are transfected and endogenous Gadd45 expressions (mRNA, and protein) are examined to determine RNAi efficiency. Two to three RNAi are designed and tested.

(viii) Site Direct Mutagenesis to Generate TR4 Phosphorylation Site Mutants.

The putative phosphorylation site on TR4 are mutated by using QuickChange XL Site-Directed Mutagenesis kit (Stratagene). pCMX-TR4 is used as a template to be amplified by two primers containing the desired mutation by PfuTurbo DNA polymerase. Following PCR cycle, the product is treated with Dpn I, which is used to digest the parental DNA template. The nicked vector incorporating the desired mutations is transformed into XL10-Gold competent cells, and clones are amplified and sequenced.

(ix) ChIP Assay:

ChIP will be carried out using the Upstate Biotechnology (Charlottesville, Va.) ChIP assay kit with modifications. In brief, TR4-transfected cells are lyzed, cross-linked with 1% formaldehyde, and chromatin pellets are sonicated to an average of 200- to 1000-bp fragments of DNA. The chromatin fragments are subjected to immunoprecipitation with 2 μg TR4 antibody overnight at 4° C. The precipitates are eluted into the elution buffer containing 1% SDS, 100 mM NaHCO₃, and 10 mM DTT. The cross-links are reversed with a 4 h incubation at 65° C. in the elution buffer with addition of 200 mM NaCl. The immunoprecipitated DNA fragments are purified using QIAGEN MiniElute Reaction Cleanup kits and subjected to PCR using a pair of primers which were designed to amplify the Gadd45a promoter sequence containing DR3-VDRE.

EMSA, and DNA pull-down assays will follow the protocols described previously (Lee, Y. F., et al. (1998) J Biol Chem, 273: 13437-13443; Bao, B. Y., et al. (2003) Adv Exp Med Biol, 543: 191-200)

(16) Examination of TR4 Effects on Promoting the Anti-Oxidative Defense Capacity.

Oxidative stress is an imbalance between the production of ROS and ability of the organism's natural protective mechanisms to cope with ROS and prevent adverse effects. It is believed that ROS is one of the primary causes of cellular damage, organic dysfunction, and aging (Droge, W. (2003) Adv Exp Med Biol, 543: 191-200). Excessive free radicals, either from endogenous sources as side products of aerobic metabolism or exogenous sources like UV and irradiation, can oxidize lipids, proteins and DNA, thus causing cell injury or even cell death (Droge, W. (2003) Adv Exp Med Biol, 543: 191-200). Higher intracellular ROS level and less scavenge abilities were seen in TR4 KO MEFs, and transfecting TR4 into TR4KO cells can restore anti-ROS effects. Thus, TR4 can promote anti-ROS defense ability by modulating the non-enzyme, low molecular weight antioxidants (LMWAs) activities and/or regulate scavenger enzymes activities. Understanding the detailed molecular mechanism of how TR4 participates in the anti-oxidant defense systems reveals not only a novel cell defense pathway involving TR4 but also provides an opportunity to promote cell defense systems by modulating TR4 activity in the cells.

(17) Determination of the Endogenous Antioxidant Enzyme Activity Including Superoxidase Dismutase (SOD), Catalase (CAT), Glutathione Peroxidase (GPx), and Glutathione Reductase (GR) in TR4 KO Vs Wt Mice Tissues.

To cope with the ROS, cells express an array of antioxidant enzymes, including Mn⁺²-dependent SOD (MnSOD), copper/zinc SOD, CAT, GPx, and GR. MnSOD and Cu/ZnSOD convert superoxide anions to hydrogen peroxide, which is then transformed to water by GPx or by CAT. It is generally accepted that the activities and capacities of antioxidant enzymes of tissue cells decline with age, leading to the gradual loss of prooxidant/antioxidant balance leading to an accumulation of oxidative damage in the aging process (Wei, Y. H. and Lee, H. C. (2002) Exp Biol Med (Maywood), 227: 671-682). TR4 KO mice develop an early onset of aging process at mid-age (5-6 month), thus TR4 KO mice antioxidant enzyme activities decline aggressively upon the aging progress. Therefore, the enzymes activities in the KO mice tissues, liver, brain and muscle, is determined and compared with their wt littermate through several segments of the life span, from neonatal (P7), before puberty (1 month), young adulthood (2-3 month), mid-age (4-6 month), mid-late (7 month to 1 yr), to late-life (over 1 year, if TR4 KO mice survive).

The amounts of scavenger enzyme expressions is quantified by Q-PCR and Western blotting analysis. The abundance of scavenge proteins is determined in the tissues from TR4 KO mice and wt at all stages. Liver, brain, and muscle from mice are harvested and then examined for their mRNA and protein expression levels by Q-PCR and Western Blotting analysis. MEFs are treated with 200 μM of H₂O₂ and vehicle control for 2 h and change the culture medium. Cells are harvested at 0 h, 4 h, 12 h, and 24 h post-treatment, and the amount of mRNA and proteins measured. The differential expression patterns are compared between with vs without H₂O₂ and TR4 KO vs wt.

(18) Determination of the Concentration of the Non-Enzyme, LMWA Like Vitamins E, C, β-Carotene, and Selenium Levels in TR4 KO Vs Wt in Blood and Tissues.

The concentration of small-molecular weight antioxidants in blood and tissue are altered, and mostly decline with advanced age. Higher intracellular ROS level and less scavenge abilities in TR4 KO MEFs raise the concern that TR4 KO have alterations in LMWA-mediated antioxidant pathways. Therefore, determining the LMWA concentration of TR4 KO and wt would provide insights towards those LMWA actions and the potential roles of how TR4 regulates those small molecules.

Here, the LMWA are determined in the TR4 KO vs wt mice serum and tissues (liver and muscle) at different stages from neonatal (P7), before puberty (1 month), young adulthood (2-3 month), mid-age (4-6 month), mid-late (7 month to 1 yr), to late-life (over 1 year, if TR4 KO mice survive).

(19) Determination of the Molecular Mechanisms Underlying TR4 Promotion of Cellular Anti-ROS Ability Via Mediating Antioxidant Enzymes Activity.

There is an age-dependent increase in ROS and free radicals suggesting that cells escape these cellular defense mechanisms as aging advances. Thus, TR4 regulates those scavenger enzymes via direct or indirect mechanisms. TR4 regulates the scavenger enzyme activities id determined according to the alterations of endogenous expression and in response to ROS. The TR4-direct scavenger targeted proteins are determined as well as the cis-acting elements located in the gene's promoters which can be bound and regulated by TR4. When no direct TR4-targeted gene is identified, then TR4 can mediate the scavenger protein activity by an indirect mechanism, such as via ROS-induced protein-protein interaction. The TR4-associated protein is identified by Co-IP.

(a) Via Direct Transcriptional Regulation by TR4:

TR4-direct scavenger targeted proteins that show altered levels of mRNA and protein in TR4 KO's tissues and MEFs (the results from 2-1), compared to wt are the first priority. Targeted gene promoters containing Luc-reporter are tested for TR4 transactivation potential in a transient transfection assay. When TR4 activates the luciferase activity, a search is conducted for the potential TR4 response element (TR4RE), which is composed of AGGTCA-like direct repeat motif in those genes' promoter, and then the direct interaction of TR4 in the targeted genes' promoter is tested. Gel shift assay, DNA-pull down, and CHIP assays is used for any potential TR4RE which is recognized and bound by TR4. Next, when no traditional TR4REs are found, serial deletion promoter reporter assays are conducted to determine the regions responsible for TR4 activity. An interaction between TR4 and transcriptional factors that bind to that TR4 responsive region is further characterized.

(b) Via Indirect Protein/Protein Interaction with TR4.

TR4-direct scavenger targeted proteins that show reduced activity in the later stage and lose H₂O₂ response in TR4 KO MEFs are candidates. FIG. 9A that SIRT1 expression level, an oxidative stress-inducible histone deacetylase longevity gene (Guarente, L. and Picard, F. (2005) Environ Res, 98: 33-39) was reduced in 6-month-old, not 3-month-old TR4 KO mice muscles. Thus, under the oxidative stress stimulation, TR4 is able to associate with stress-responsive factors, such as SIRT1 to promote cellular anti-ROS defense systems. To confirm this, cells are challenged with oxidative stresses, and TR4-associated proteins will be pulled down by CoIP with TR4 antibody. The TR4-associated immunocomplex is analyzed by SDS-PAGE. Differential protein expression when compared between non-stress and stress-treated cells is selected. In screening stress induced-TR4 interaction proteins, SIRT1 is one of the TR4-associated factors, and TR4 is one of substrates deacetylated by SIRT1. The interaction domain is narrowed down and potential interaction sites on TR4 are mutated to eliminate the interaction. Functional tests of this association between TR4 and stress-induced factors are further tested using TR4 mutants, or overexpression of small interaction-peptides to interrupt the interaction, and examine cellular scavenger ability changes.

(20) Determination of the Molecular Mechanisms Underlying TR4 Promotion of Cellular Anti-ROS Ability Via Mediating LMWA Activity.

To determine if TR4 is a mediator for non-enzymatic antioxidants, including vitamin C, vitamin E, β-carotene, and selenium, to suppress ROS production in the cells, cellular ROS levels are examined in LMWA-treated MEFs to antagonize oxidative stress. MEFs from TR4 KO and wt are treated with LMWA with/without H₂O₂ oxidative stress challenge as ROS levels are measured. The ones that lose its anti-oxidant activity in KO MEFs but not wt MEFs are the candidates. Herein, it was found that 10⁻⁷ M vitamin E can reduce wt but not KO MEFs intrinsic and exogenous ROS production indicating that TR4 KO MEFs have defects in vitamin E-mediated anti-oxidant ability. Therefore, TR4 is mediated by some LMWAs, such as vitamin E, to suppress cellular ROS production. Herein, the anti-ROS effects are compared on TR4 KO vs wt MEFs in response to other LMWAs. The focus is on how TR4 regulates vitamin E and the other LMWA that lost anti-ROS response in TR4 KO MEFs.

(a) Screening Other LMWA Anti-ROS Effects on TR4 KO Vs Wt MEFs.

MEFs (TR4 KO vs wt) from P1 to P4 are used for the screening. Cells are treated with H₂O₂ or vehicle, in the presence and absence of different concentrations of LMWAs (vitamin C, β-carotene, and selenium) and then cellular ROS levels are determined. The difference between TR4 KO and wt are compared for the intrinsic ROS (before H₂O₂ challenge) and extrinsic ROS (after H₂O₂ challenge) levels after LMWA treatments. The ones that show defects in TR4 KO will be further investigated (Genova, M. L., et al. (2004) Ann N Y Acad Sci, 1011: 86-100). Investigation of the molecular mechanisms by which TR4 promotes LMWA (such as vitamin E)-mediated anti-ROS defense systems. Vitamin E is a small steroid-like compound, and the heterogeneity of mediators of vitamin E action suggests there is a common element that there is a receptor or a co-receptor, able to interact with vitamin E and with transcription factors directed toward specific regions of promoter sequences of sensitive genes. Disclosed herein, TR4 promotes vitamin E anti-ROS actions via interacting with vitamin E in the cells (facilitating uptake, preventing degradation, or directly binding). To examine TR4 effects on the vitamin E stability in the cells, vitamin E levels are measured in the TR4- and vector-transfected cells when treated with vitamin E and vehicle control at different time points. When TR4-transfected cells have higher vitamin E levels or longer half-life, how TR4 affects vitamin E uptake/degradation pathways is examined by examining the potential cross-talk with vitamin E transfer proteins, such as tocopherol-associated proteins (TPA), or cytochrome p450 (CYP)-dependent hydroxylases, such as CYP3A family. TR4 is a nuclear orphan receptor and loss of TR4 results in impairment of vitamin E anti-ROS function. Therefore TR4 can be a receptor or co-receptor for vitamin E anti-oxidant effects. To examine if TR4 directly associate with vitamin E, TR4RE-Luc reporter genes assays are performed. Different TR4RE reporters are tested in different cell lines under the oxidative stress and/or combination with vitamin E treatments (dose from 1 nM to 1 μM). When vitamin E by itself can activate TR4 reporter genes, then binding affinity (Kd) is determined by Schartchard Plot analysis; in contrast, when vitamin E activates TR4 only in the presence of oxidative stress (H₂O₂), that indicates TR4 associates with vitamin E in an indirect oxidative stress dependent manner.

Similar approaches are applied with other LMWAs (vitamin C, β-carotene, and selenium) when other LWWA-mediated anti-ROS defects are identified in TR4KO MEFs.

(b) Detailed Methods:

(i) Determination of SOD, CAT, and GPX, Protein Expression Level, and Activity:

Tissue preparation: Muscle, liver, and brain from TR4 KO vs wt mice at different ages are collected. Protein extracts are prepared by homogenizing mouse tissues in a buffer of 50 mM sodium phosphate/100 mM NaCl/1% Nonidet P40. The protein concentration is determined by a standard BCA assay.

(ii) Immunoblotting of SOD, CAT, and GPX, Proteins.

Total cellular protein (20 μg) is electrophoresed in 4-20% tris-glycine sodium dodecyl sulfate polyacrylamide gels (Novex, San Diego, Calif.). Proteins are transferred onto polyvinyl diethyl fluoride membranes (Millipore Corp., Bedford, Mass.), blocked in 5% dry milk in T-TBS (0.02 M Tris/0.15 M NaCl, pH 7.5 containing 0.1% Tween 20) at room temperature (RT) for 3 h, washed three times with T-TBS, and incubated with the primary antibodies to the Cu/Zn SOD (1:2000, Calbiochem), Mn SOD (1:1000, Calbiochem), CAT (1:2000, Calbiochem), GR and GPX (1:250, Cortex) for 3 h at room temperature. After washing five times with T-TBS, the blots are incubated with secondary antibodies (anti-sheep for Cu/Zn SOD, Mn SOD, and GPX, 1:2000, anti-rabbit for CAT, 1:2000, and GC 1:1000) conjugated with horseradish peroxidase at RT for 2 h. After being washed five times with T-TBS, the membranes are developed using enhanced chemiluminescent reagent (Amersham Life Science Inc., Arlington Heights, Ill., USA) and subjected to autoluminography for 1-5 min. The autoluminographs are scanned with a laser densitometer to determine the relative optical densities of the bands (Farmand, F., et al. (2005) Environ Res, 98: 33-39).

(iii) Determination of Cu/Zn SOD, CAT, GR, and GPX Activities:

Cu/Zn SOD, GR and GPX activities were determined by using Bioxytech SOD-525 and Bioxytech GPx-525 kits, respectively, purchased from OXIS International, Inc. (Portland, Oreg.) according to the manufacturer's directions. CAT activity is measured by determining the decomposition of its substrate H₂O₂ as described by Claiborne (Claiborne, A. Catalase activity. Boca Raton, Fla.: CRS Press, 1985)

(iv) Determination of LMWA Concentration in Blood and Tissues from TR4KO and Wt at Different Ages.

(a) Vitamin E.

One hundred μl of serum or tissue extracts are added with 0.3 ml 1% ascorbate plus internal standard 1 mmol δ-tocopherol and 0.4 ml ethanol. Vitamin E isoforms are extracted with 0.8 ml hexane. The hexane extract is taken to dryness under N2 in TURBOVAP®LV concentration workstation (Zymark, MA), residue dissolved in 2.5% ascorbate in methanol (1 ml), and analyzed (50 μl) by HPLC. Measurements of Vit E ether analog is made on Spherisorb ODS II column (250×4.6 mm I.D. 5 μm; Waters) and eluted with methanol:isopropanol (99:1; V/V) at a flow rate 1 ml/min. The signals are detected by Waters 2475 multi λ fluorescence detector. Excitation and emission wavelengths are 295 nm and 330 nm.

(b) Vitamin C:

The serum vitamin C concentration is determined by the 2,3-dinitrophenylhydrazine method with calorimetric analysis. Immediately after separation by centrifugation, serum is deproteinized, and the supernatant stored at −20° C., and measurements completed within 10 days (Yokoyama, T., et al. (2000) Stroke, 31: 2287-2294).

(c) β-Carotene:

β-carotene is converted to vitamin A (retinol) by the body. While excessive amounts of vitamin A in supplement form can be toxic, the body will only convert as much vitamin A from β-carotene as it needs. To a 0.1-ml sample, 0.68 ml ddH2O, 20 μl of 50 g/l BHT (dissolved in ethanol), 70 μl trifluoroacetic acid, and 0.5 ml of 14 g/l thiobarbituric acid will be added. The mixtures are incubated at 95° C. for 45 min. Following a 10-min centrifugation (1000×g), the samples are determined by reversed-phase HPLC using a Suplex pKb 100 column (5 μm, 250×4.6 mm, Supelco) and detected with photodiode array detector at 450 nm as described previously.

(d) Selenium (Se):

Se levels are determined by the fluorometry with 2,3-diaminonaphthalene (DAN) follow the published protocol (Zachara, B. A., et al. (2001) Early Hum Dev, 63: 103-111). Tissue or blood samples are placed in a flask with 10 ml concentrated nitric acid and allowed to stand for 24 h at room temperature. Four milliliters of 72% perchloric acid is added for digestion at high temperature. After digestion all traces of nitric acid were removed by heating. The reduction of selenate to selenite, by the addition of HCl, formation of DAN-Se complex, and extraction of the complex into cyclohexane is performed. The fluorescence is measured on a spectrofluorimeter.

RNA preparation, RT-PCR, Q-PCR EMSA, DNA pull-down, and CHIP assays were described previously.

(21) Determination of how TR4 is Involved in DNA Repair System to Maintain the DNA Integrity.

Results from TR4 KO studies indicated that cells lacking TR4 have a higher number of DNA breaks than wt and TR4 KO cells are more sensitive to DNA-damage stress, which indicated a role of TR4 in the DNA repair pathways. Furthermore, in vitro DNA repair reporter gene assay demonstrated that TR4 can induce repair of UV-induced DNA damage. Together, this indicates that TR4 functions in the cell as a DNA damage sensor, which conveys stress-induced information via biochemical signal transduction pathways in the execution of DNA repair. Therefore, TR4 KO cells are unable to detect cellular damage and lack the ability to recruit DNA repair machinery to the sites of DNA lesions early in response to damage. The functions of TR4 on DNA repair in response to DNA-damage inducers are studied, as well as the temporal regulation of this process. The ability of TR4 to influence various DNA repair capacities, and the functions of TR4 in DNA repair signal transduction either via the direct regulation or via protein/protein interactions, is addressed. TR4 KO, wt, or TR4-transfected KO cells are subjected to UV and IR to study how TR4 induces DNA repair signals, including nucleotide excision (NER), homologous recombination (HR), and nonhomologous end jointing (NHEJ). The methods disclosed herein not only identify a novel function of TR4 in DNA repair pathway to prevent cellular decay, but also provide a novel strategy against un-repaired cellular and molecular damages through modulation of TR4 activity.

(22) To Characterize the Relationship Between Double Strand Break DNA Repair (DSBR), Fidelity, and Radiation Sensitivity, and to Determine if this is a Result of TR4 Expression.

Double-strand DNA breaks (DSBs), the most genotoxic lesions, are mostly caused by ROS and ionizing irradiation. It is extremely important for cells to repair this a kind of damage as DSBs are susceptible to exonucleases, leading to loss of large genomic regions. If these lesions are improperly processed, they result in the accumulation of genetic mutations, and lead to carcinogenesis. Accordingly, mammalian cells have adapted protective mechanisms to counteract the harmful effects of IR-induced DSBs, mainly the evolution of several distinct, non-overlapping repair processes. Three repair pathways are involved in repairing DSBs: a true repair by homologous recombination (HR), a less accurate repair by non-homologous end joining (NHEJ), and single-strand annealing (SSA), a transitional pathway between HR and NHEJ. IR-induced radiation sensitivity is governed by a series of factors, including regulation between cell cycle checkpoints, apoptosis, and DNA repair. It is disclosed herein that TR4 is involved in regulation of DSBR activity.

(a) End-Joint Assay:

Data indicated that TR4 is IR-induced and involved in radiation sensitivity. It is disclosed herein that TR4 KO cells exhibit decreased fidelity in repair due to decreased HRR/NHEJ. Decreased fidelity in repair leads to increased radiation sensitivity as damaged cells undergo cell death. Therefore, fidelity in DSBR is measured by End-joint assay to determine if a difference in repair between TR4 KO and wt (KO with TR4 transfection) cells can be discerned, contributing to radiation survival. First, the ability of wt vs TR4 KO nuclear extracts to rejoin DSBs introduced into the lacZ gene of plasmid DNA is tested, thereby restoring expression of β-galactosidase. β-galactosidase activity can be measured by blue colony formation on X-gal plates.

(b) Differential expression of proteins involved in DSBR in TR4 KO vs wt by Western blotting analysis.

The interaction of TR4 with proteins involved in HR, DNA-PK, and other NHEJ activates the DNA repair machinery. Expression and activity of factors involved in both HR and NHEJ are characterized TR4 in DSBR. Proteins are extracted from TR4 KO and wt (or KO transfected with TR4) MEFs at 0, 3, 6, 9, and 12 h post 8Gy irradiation, and analyzed for the expression of Rad52, Rad54, Rad51, DNA-PKcs, Ku70, and Ku86 by Western blotting analysis.

(c) DNA-PK Activity:

To further characterize the repair capacity in TR4 KO and wt, the activity of DNA-PK and its ability to phosphorylate known substrate p53 is assessed (Douglas, P., et al. (2001) J Biol Chem, 276: 18992-18998; Bharti, A., et al. (1998) Mol Cell Biol, 18: 6719-6728). In vitro kinase reactions are performed as previously described (Brown, K. D., et al. (2000) J Biol Chem, 275: 6651-6656; Kurimasa, A., et al. (1999) Mol Cell Biol, 19: 3877-3884). Briefly, DNA-PK from cells (TR4KO and wt) treated with 8Gy irradiation are collected at 0, 3, 6, 9, 12 hs post-irradiation, and pulled-down by immunoprecipitation. Kinase reactions are carried out by adding purified recombinant GST-p53 protein, 5 μM cold ATP, 30 μM Ci γ 32PATP, and 500 ng of sonicated salmon sperm DNA to the slurry of beads containing immunoprecipitated DNA-PKs. This reaction is incubated for 30 min and terminated by adding an equal volume of 3×SDS sample buffer. The final protein products are resolved in 8% SDS-PAGE, and dried. The kinase activity will be quantified by phosphorimaging.

(d) Rad51 Co-Localization.

It is disclosed herein that TR4 binds DSBs and interacts with HR/NHEJ machinery to activate the repair process. To determine if this interaction occurs at the site of DNA damage, three color microscopy is performed to observe the co-localization of Rad51 and TR4 at strand breaks in response to treatment with IR. Strand breaks leaving 3′ hydroxyl ends can be labeled with bromylated deoxyuridine triphosphate nucleotides (3r-dUTP) by using the terminal deoxynucleotidyl transferase (TdT) enzyme. Double staining with Rad51 allows the direct visualization of cells that have accumulated Rad51 at the site of DNA damage. TR4 KO and wt (or TR4 KO with TR4 transfection) cells are fixed at 0, 3, 6, 9, 12, and 24 h post 8Gy IR. For the detection of strand breaks, fixed nuclei are incubated with mouse or rat anti-BrdU (Becton Dickinson, Serolab). Cells are stained with anti-TR4 (#15), rabbit anti-Rad51, and anti-Rad52 (Santa Cruz Biotechnology), and rat anti-BrdU (Serolab). Secondary antibodies containing fluorescent conjugates to PE, FITC and Cy3 are available to each primary antibody (Jackon Labs, Becton Dickinson). Immunofluorescence is recorded using a confocal microscope. In addition, DAPI counterstaining is performed following washing with PBS (Haaf, T., et al. (1999) J Cell Biol, 144: 11-20). The colocalization and interaction of TR4 with Rad51 at DSBs is indicative of suppression of HRR.

(e) Co-Immunoprecipitation Analysis.

To determine if TR4 is involved in protein/protein interactions with DNA repair machinery, cell lysates (from TR4 KO and wt) and immuno-precipitations are prepared following 8Gy IR treatment. 150 μg protein extracts are incubated with anti-DNA-PK, anti-p53, or anti-Rad51 for 2 h at 4° C. Immune complexes are precipitated with Protein A-sepharose beads and the immunoprecipitates are resolved by SDS-PAGE, and transferred to nitrocellulose. The filters are immunoblotted with anti-Ku70, -Ku80, -DNA-PK, -Rad51, -Rad52, -Rad54, and -TR4. The antibody complexes are visualized by enhanced chemiluninescnce. The data show which proteins are specifically bound to DNA in TR4 KO and wt cells.

(23) To Determine the Roles of TR4 in UV-Damaged DNA Repair.

TR4 mediates DNA repair in response to UV-induced DNA damage. Therefore, TR4 modulates the NER pathway via direct regulation of the factors involved in NER and/or indirectly affecting NER pathway through interacting with NER factors (NERFs). Nucleotide excision repair (NER) pathways can be modulated by TR4 upon the UV-treatment.

(a) Identify the TR4 Direct Target Genes in NER Pathways.

TR4-targeted proteins involved in NER pathways are identified by comparison the differential expression of proteins in TR4 KO vs wt by Q-PCR and Western blotting analysis. The repair genes including Xpa (initiates repair), Xpb and Xpd (helicase to unwind DNA), XpF/ERCC1 and Xpg (endonucleases cleave DNA) are examined. Proteins are extracted from TR4 KO, wt and TR4 KO transfected with TR4 MEFs at 0, 3, 6, 9, and 12 h post-UV irradiation (100 J/m²), and analyzed for the expression of those proteins involved in NER systems. The factors that are reduced in TR4 KO cells, compared to wt cells, are the TR4 target genes and will be further investigated in the context of how TR4, as a transcriptional factor, regulates those genes activities. The target genes' 5′-promoter containing Luc reporters are requested (if available) or cloned and tested in a transient transfection assay with overexpression of TR4. When TR4 activates Luc gene activity, the responsive element located in the genes 5′-promoter is identified. Putative TR4RE (AGGTCA-like direct repeat motif) is searched for if the sequences are known; if not, 5′-promoter deletion mutations are constructed to locate the region responsible for TR4 regulation. Once the putative TR4RE is targeted, EMSA, DNA-pull down, and ChIP assays are performed (detailed methods described previously).

(b) Identify the UV-Induced TR4 Interacting Proteins Involved in NER Pathways.

TR4 can regulate the NER pathway via indirect mechanisms. The genes that lost UV-responsiveness in TR4KO cells are genes regulated by TR4 via indirectly mechanisms, such as protein/protein interactions. To test this, the CoIP is performed to precipitate TR4-interacting complex from UV-treated cells, and plot with antibodies against the proteins involving the NER system. The factors that show UV-induced interaction with TR4 are examined in the context of how this interaction (TR4 and factors) influences the NER pathway. When TR4-interacting proteins are found, the interaction domain in TR4 is identified, and tested to see if interrupting the interaction results in impairment of NER pathway using over-expression of small-interacting peptides.

(c) Test the Protein/Protein Interaction.

To test the interaction between TR4 with NER factors, mammalian two-hybrid system (GAL4DBD-TR4 and VP16-NERs) or GST-pull down assays are used. In addition to in vitro interaction, colocalization between TR4 and NER factors from UV-treated vs un-treated cells are performed by double staining with anti-TR4 and anti-NER and then visualizing incubated different secondary antibodies-conjugated with FITC or Cy3.

(d) Identify the Interacting Domain in TR4.

To narrow-down the interacting domain in the TR4, Gal4 DBD constructs containing full length, N-terminal, DNA-binding domain, and ligand-binding domain of TR4 (GAL4 DBD-TR4-f, GAL4DBD-TR4-N, GAL4 DBD-TR4-DBD, GAL4DBD-TR4-LBD) are tested for their interaction with VP-16-NERF using mammalian two hybrid assay.

(e) Interrupting the TR4 Interaction with NERFs.

The TR4 interacting peptide identified in (B) is transfected into the cells, and then UV-damaged DNA repair assays (Luciferase-based DNA repair measurement and Q-PCR-based DNA repair measurement) are performed to see if interrupting the interaction can affect the DNA repair capacity.

(f) Detailed Methods

(i) UV-Damaged DNA Repair Assay

(ii) Luciferase-Based DNA Repair Measurement:

Cells are transfected with 0.5 μg of the CMV-luciferase damaged by 5000 J/m² of UV irradiation to induce DNA damage and 0.1 μg of the undamaged CMV-renilla, and treated with virus supernatants of the pBabe-TR4 transfected cells or the pBabe transfected cells. After 24 h transfection, luciferase assays are performed. DNA repair will be assayed by the luciferase activities. CMV-luciferase activities are normalized to that of CMV-renilla. Fold repair is calculated by dividing the normalized luciferase activities by that of the empty vector.

(iii) Q-PCR-Based DNA Repair Measurement:

Cells are transfected with 0.5 μg of the pBluescript vector (Stratagene) damaged by 5000 J/m2 of UV irradiation, 0.1 μg of the undamaged pGL3-Basic vector (Promega), and 0.4 μg of the pBabe-TR4 construct or 0.4 μg of the pBabe/pure (an empty vector). DNA repairs assayed by quantitative real-time PCR using T3 and T7 primers for the pBluescript vector, and GL2 and RV3 primers for the pGL3-Basic vector. pBluescript PCR quantities are normalized to pGL3-Basic PCR quantities. Fold repair is calculated from the normalized PCR quantities divided by those of the empty vector.

(24) Elucidation of the Roles of TR4 in Controlling the Cell Senescence Vs Cellular Oncogenic Pathways In Vitro and In Vivo.

Cellular senescence is a response to stress, such as DNA damage, oxidative stress, oncogenic activity, and plays an important role in tumor suppression and contributes to organism aging (Campisi, J. et al. (2005) Cell, 120: 513-522; Neumeister, P., et al. (2002) Int J Biochem Cell Biol, 34: 1475-1490). Lack of TR4 in mice results in accelerated aging, and early onset of premature cellular senescence strongly supports the TR4 role in guarding genome stability via both reducing oxidative stress and promoting DNA repair capacity. It has become clear that loss of genome stability due to malfunctioning DNA repair machineries can have catastrophic consequences, such as cancers and premature aging. The studies identify TR4 as a key regulator in the ROS-DNA damage-DNA repair cascade that not only links TR4 to these genomic maintaining networks, but also indicates that TR4 participates directly in making life and death decisions in the cells. Disclosed herein, TR4 can regulate cellular response to oncogenic stress in which TR4 can prevent cells from being senescent/transformed when cells undergo oncogenic insults. Tumorigenic conversion of primary fibroblast requires at least two cooperating oncogenes, or in combination of inactivation of tumor suppressor genes (Weinberg, R. A. (1983) J Cell Biol, 97: 1661-1662; Land, H., et al. (1983) Nature, 304: 596-602; Ben-Porath, I. and Weinberg, R. A. The (2005) Int J Biochem Cell Biol, 37: 961-976). The cellular response to Ras-oncogenic insults are examined by altering TR4 amount in the cells using knockdown (TR4 KO or TR4 RNAi) and overexpression of TR4 in SV40LT immortalized MEFs cells, to gain further insight into the mechanisms of TR4 effects on the cellular response to oncogenic stress TR4 anti-ROS and anti-DNA damage effects can be confirmed in the human fibroblast cells WI-38 and WI-38 immortalized with SV40-TAg in vitro and in vivo. The goal is to further confirm TR4 roles in guarding genomic integrity (via sensing stress, anti-ROS, and DNA repair) by introducing Ras to the cells, which would induce cell growth arrest or cell transformation and to see if TR4 can alter the cell fate by preventing DNA damages.

(25) Examine the Effects of TR4 on Cellular Senescence in Human Fibroblast Cells.

(a) Loss of TR4 Shortened the MEFs Lifespan in which Most of the Cells Arrest in G2/M, Supporting the Roles of TR4 in Guarding the Genomic Integrity.

Cellular senescence is a program executed by cells in response to a variety of stresses, including DNA damage, oxidative stress, oncogene activity, and others (Ben-Porath, I. and Weinberg, R. A. The (2005) Int J Biochem Cell Biol, 37: 961-976; Serrano, M., et al. (1997) Cell, 88: 593-602; Rangarajan, A., et al. (2004) Cancer Cell, 6: 171-183). Laboratory mice have represented a powerful experimental system for understanding the intricacy of human cancer pathogenesis. Indeed, much of the current conceptualization of how tumorigenesis occurs in humans is strongly influenced by mouse models of cancer development. However, an emerging body of evidence indicates that there are fundamental differences in how the process of tumorigenesis occurs between mice and humans. Human TR4 RNAi are transfected into WI-38 cells, then selected with puromycin, and the clones containing TR4 RNAi are further characterized for the cell morphology, growth rate, ROS response, DNA damage, senescence-associated-β-gal staining, and cell cycle profiles analyses. Cell senescence genes, such as p53, p16, p21, Rb are compared.

(26) Determine the Roles of TR4 on the Ras-Mediated Cell Transformation in the Immortalized MEFs In Vitro and In Vivo.

It has been demonstrated that perturbation of two signaling pathways involving p53 and Raf suffice for the tumorigenic conversion of normal murine fibroblasts. As indicated herein, TR4 serves as a guard to protect the cells from genotoxic stress. TR4 can prevent the cells from Ras-mediated oncogenic pathway in SV40LT immortalized MEFs with inactivated p53. It is disclosed herein that TR4 not only protects cells from ROS-induced DNA damage-mediated cell senescence, but also prevents cells against cellular tumorigenic transformation. MEFs from wt and TR4 KO are immortalized by SV40LT and then introduce retroviral vector encoding Ras oncogene (pWZL hygro-H-RasV12) and vector control to induce cell transformation. The immortalized MEFs from TR4 KO and wt are examined for their cell transformation capacity judging by cell morphology, cell growth, DNA damage degree, cell cycle profile, and soft agar assays. Finally, the MEFs/human fibroblast showing anchorage-independent growth in soft agar is used to confirm tumorigenicity in nude mice xenografts.

(27) Determine the Roles of TR4 on the Ras-Mediated Cell Senescence Vs Cell Transformation in the Immortalized Human Fibroblast In Vitro and In Vivo.

Expression of multiple oncogenes and inactivation of tumor suppressors is required to transform primary mammalian cells into cancer cells. Recent evidence suggests that human cells require more genetic changes for cell transformation than do their murine counterparts. Activation of Ras is usually associated with cancer; however, it produces premature senescence in primary immortalized human fibroblasts. Herein, the ability of the modulation of TR4 expression in these Ras-activated immortalized human fibroblasts (WI-38 VA-13, ATCC) to bypass Ras-induced senescence and allow Ras to transform these cells is tested. Stable cells with different degrees of TR4 expression levels (normal, knockdown, and overexpression) are examined to determine the cell characteristics. If cells can grow in soft agar, they are further tested for their in vivo tumorigenicity in nude mice xenografts.

(a) Detailed Methods

(i) Generation of MEFs.

Mating is set up by using TR4 heterozygous male and female, and checking for vaginal plugs every early morning. Once obtaining an E14.5 female, the mouse is sacrificed and dipped in 70% ethanol. Embryos are collected, and placed in a sterile Petri dish with PBS, remove heads and all the internal organs (liver, heart, kidney, lung, and intestine) from embryos and after that wash with PBS twice. Place the tissues in 5 ml of DMEM culture medium, pass it through a 22 gauge needle a few times. Transfer the minced tissue into a 25 cm² tissue culture flask which contains 10 ml medium, and culture overnight at 37° (5% CO₂). Change the medium after 25 h to remove unattached cells and debris. After 2 or 3 days of culture the MEF cells form a confluent monolayer; then trypsinized each plate and split them ⅕.

(ii) Retroviral-Mediated Gene Transfer:

To restore TR4 expression in TR4 KO MEF cell, pBabe-hTR4 are used for retroviral infection. Ecotropic packaging cells will be plated for 24 h and then transfected with SuperFect (Quigen) with pBabe-pur/2 or pBabe-TR4. After 48 h the viral containing medium is filtered (0.45 mM filter, Millipore) to obtain viral-containing supernatant. Targeted MEF cells are plated and the culture medium is replaced with a mix of the viral-containing supernatant and culture medium, supplemented with 4 μg/ml polybrene, and the cells are incubated at 37° C. MEF cells infected with the empty vector (pBabe-puro) are used as control.

(iii) Immortalization of MEFs:

Once MEFs cells are generated, MEFs are immortalized by expressing SV40LT in the MEFs. Viral vector encoding SV40LT cDNA(pBabe-SV40LT) is transduced with retrovirus and purified with puromycin (1-2 μg/ml) starting 1 or 2 days after infection for 4 days.

(iv) Ras Oncogene Overexpression.

The immortalized MEFs and human fibroblast WI-38 VA-13 cells are infected with retrovirus containing Ras oncogene (pWZL hygro-H-RasV12). To eliminate the uninfected population, cells are selected with hygromycin B (100 μg/ml), starting 1 or 2 days after infection for 4 days. After selection is complete, cells are examined for expression/activity of Ras and its down-stream activators, such as Raf, MEKK, and ERK. The cells then are tested for the tumorigenicity by anchorage-independent growth assay.

(v) Senescence-Associated β-Galactosidase Staining:

Cells are washed with PBS and fixed for 5 min in 2% formaldehyde and 0.2% glutaraldehyde. Fixed cells are washed with PBS and incubated with fresh senescence-associated β-galactosidase stain solution (sodium phosphate buffer, pH 6.0 containing 1 mg of X-gal/ml 40 mM citric acid, 5 mM potassium ferrocyanide, 5 mM potassium ferricyanide, 150 mM NaCl, 2 mM MgCl2). Staining is detected by light microscopy following overnight incubation.

(vi) Anchorage-Independent Growth Assay:

Soft agar assays are performed as described previously. Briefly, individual cell lines are seeded in triplicate at three different dilutions ranging between 1×10³ and 5×10⁵. Each experiment is repeated at least once. Colonies are counted and photographed between 18-24 days under phase contrast microscopy.

(vii) Tumorigenicity Assay:

6-8 week old athymic nude mice are injected subcutaneously, bilaterally into dorsal-lateral flanks with 0.2 mixture of 2×10⁶ cells mixed with Matrigel (v:v=1:1). The growth of tumors is monitored by measuring the tumor volume. Tumor volume is calculated as v=L×1²×0.52, where L and 1 represent the larger and the smaller tumor diameter, respectively, measured daily.

Western, RT-RCR, Real-time PCR follow the protocols described previously.

### Example 2

### TR4 and Cancer Pattern

Testicular Orphan Receptor 4 (TR4) is responsible for maintaining the genomic stability in which lack of TR4 results in accelerated aging in mice and early onset of cellular senescence in the mouse embryonic fibroblasts (MEF). Further mechanistic studies indicate that TR4 is able to mediate cellular response to DNA-damage signals by sensing stress, blocking reactive oxygen species (ROS), as well as regulating DNA repair networks, all of which are counteract tumor promotion. Tissue microarray (TMA) analysis of prostate samples reveals a strong correlation of TR4 expression with the prostate cancer progression. Strikingly, TR4 signals shifted dramatically from nucleus to cytoplasm with the progression of the disease. Based on these data, it is shown herein that TR4 is a stress sensitive “caretaker gene” that inhibits mutation(s) by blocking ROS, preventing/repairing DNA damage and dysfunction of TR4 results in cancer if activated oncogenes or inactivated tumor suppressor genes are involved.

a) DNA Repair Factors in Aging and Cancer:

Accumulation of somatic mutations has long been considered as a major cause of aging and aged-related diseases such as cancer. Genomic rearrangement, that arise from aberrant DNA breaks are often found in aging cells and tissues. Deficient/mutation in DNA repair networks is likely to result in accelerated progression of aging and an increased risk of cancer both of which shown in many mouse and human models. Patients with Werner syndrome caused by a mutation in a RecQ DNA helicae/nuclease (WRN) display premature aging and an increased risk of cancer (Chen L, et al. 2003 Aging Cell 2:191-9; Nakayama H 2002 Oncogene 21:9008-21; Epstein C J, Motulsky A G 1996 Bioessays 18:1025-7). Cultured somatic cells from WRN patients have shorten replicative lifespan with increased rates of genome rearrangement. Knockout of Ku 80, a DSB repair gene, causes premature aging and cancer (Difilippantonio M J, et al. 2000 Nature 404:510-4). Disruptions of mice DNA-PKs, a key component of non-homologous end-joint (NHEJ) pathway result in a shorter lifespan and show early onset of lymphoma (Espejel S, et al. 2004 EMBO Rep 5:503-9). Mutation in Atm gene in human results in the genetic disorder with both premature aging and cancer predisposition (Shiloh Y, Kastan MB 2001 Adv Cancer Res 83:209-54).

b) Oncogene Cooperation and Multiplestep Carcinogenesis:

In response to activation of oncogenes or DNA damage stresses, checkpoint proteins trigger mechanisms such as apoptosis or senescence to terminate pre-malignant condition (Serrano M, et al. 1997 Cell 88:593-602). Therefore, cellular senescence is thought to suppress tumor development by establishing a growth arrest that requires activities of p53 and pRB. Transformation of primary cells by oncogenes, such as Ras, require other cooperation alteration to the cells, such as overexpressed activation of other oncogenes or inactivation of tumor suppressor genes, such as p53 or p16 (Schmitt C A, et al. 2002 Cell 109:335-46). Cell-cycle arrest in response to oncogene ras may provide selective pressure to mutate p53 and p16 during carcinogenesis Consistent with this view, mutation and amplification of ras and loss of p16 and p53 are extremely common in human pancreatic cancer and colon cancer (Ben-Porath I, Weinberg R A 2004 J Clin Invest 113:8-13; Bertoni-Freddari C, et al. 1994 Ann N Y Acad Sci 717:13749)

c) Tumor Suppressor Mechanisms:

Epidemiological data provide evidence that it is possible to prevent cancer and other chronic diseases, through avoidance of exposure to recognized risk factors that induce common pathogenetic mechanisms, such as DNA damage, oxidative stress, and chronic inflammation. In a primary setting of prevention, it is possible to inhibit mutation and cancer initiation by triggering protective mechanisms either in the extracellular or intracellular environment, e.g., by modifying transmembrane transport, blocking reactive species, inhibiting cell replication, maintaining DNA structure, modulating DNA metabolism and repair, and controlling gene expression. DNA damage response can be an anti-cancer barrier in early stage of tumor development (Bartkova J, et al. 2005 Nature 434:864-70). Tumor promotion can be counteracted by inhibiting genotoxic effects, favoring antioxidant and anti-inflammatory activities, inhibiting proteases and cell proliferation, inducing cell differentiation, modulating apoptosis and signal transduction pathways, and protecting intercellular communications. In a secondary line of prevention, while premalignant/malignant lesions have been detected, it is also possible to inhibit tumor progression via the same mechanisms. There are two kinds of tumor suppressor genes: gatekeepers and caretakers, which are responsible for guarding the genome integrity and avoiding accumulation/amplification of DNA mutations and chromosome aberrations. Gatekeeper genes act directly to regulate cell proliferation, are rate limited for tumorigenesis, and examples being the retinoblastoma (RB) and p53 genes. Gatekeeper genes can prevent cancer by inducing programmed cell death (apoptosis) or permanent withdrawal from the cell cycle (senescence) to eliminate potential cancer cells from more dangerous mutations turning cells into fully fledged cancer. Caretaker tumor suppressor genes, by contrast, do not directly regulate proliferation, instead they defend genome integrity by preventing DNA damage and/or optimizing DNA repair(Levitt N C, Hickson ID 2002 Trends Mol Med 8:179-86). Since mutations not only cause cancer, but also contribute to aging, caretaker genes, in essence, are longevity assurance genes. Mutations/malfunction of both kinds of tumor suppressor genes leads to accelerated conversion of normal cells to neoplastic cells.

d) TR4 Regulates Nonhomologous End Jointing (NHEJ).

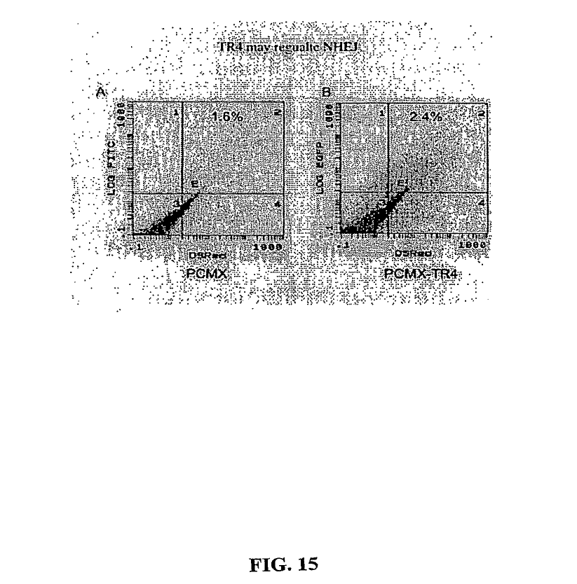

To test whether TR4 is involved in NHEJ in response to DSBs, a newly developed NHEJ assay system was used (Seluanov A, et al. 2004 Proc Natl Acad Sci USA 101:7624-9). As shown in FIG. 15, cells expressing TR4 had a slightly higher NHEJ activity than the control cells expressing empty vector only.

e) Construction of TR4 RNAi:

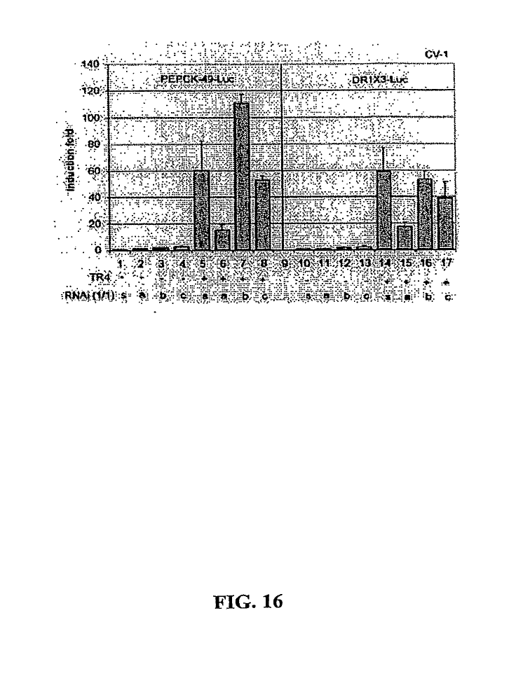

As shown in FIG. 16, two TR4 RNAis, pRetro-TR4 RNA-a and pRetro-TR4 RNA-c, but not pRetro-TR4 RNA-b can suppress TR4-mediated reporter gene activity (PEPCK-Luc, and DR1×3-Luc).

f) Prostate Hyperplasia Found in 17 Month Old TR4 KO Mice:

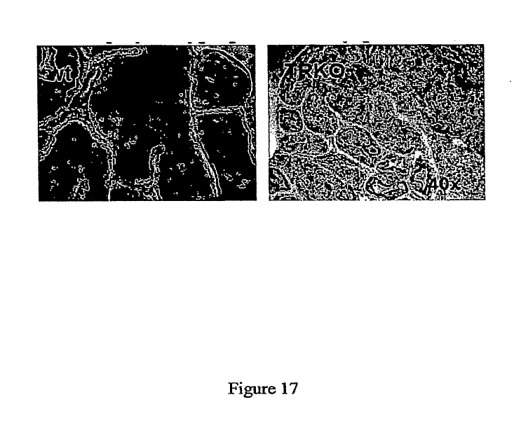

Elimination of TR4 resulted in elevated cellular ROS and induced early onset cell cycle arrest; eventually cells might bypass senescence to develop cancer, if assaults continue. Therefore, we examined if there were any abnormalities occurring in the late-life stage of TR4 KO mice. As shown in FIG. 17, we found that the ventral prostate (VP) from TR4 KO displayed hyperplasia and dysplasia at 17-months.

g) Elevated and Abnormal TR4 Expression in Prostate Cancer tissues.

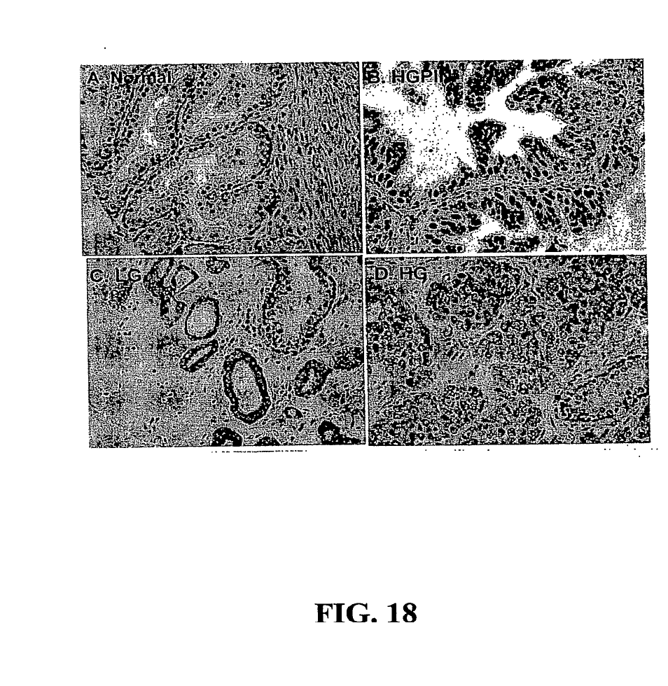

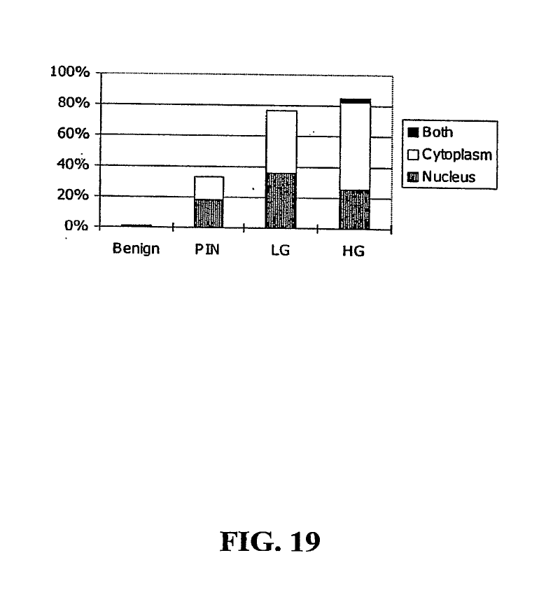

To understand the roles of TR4 in prostate cancer, TMA analysis in a large number of prostate carcinoma cases of TR4 expression were performed. Five different types of prostatic tissue were examined including: normal (N), benign hyperplastic (BPH), prostatic intraepithelial neoplasia (PIN), low-grade adenocarcinoma (LG), and high-grade adenocarcinoma (HG). As demonstrated in FIG. 18, TR4 expresses mainly in the nucleus of basal cells of normal prostate (A), however, with much stronger staining of both nucleus and cytoplasm in PIN (13) and LG (C) and HG (D) tumor. After reviewing and scoring a strong correlation of TR4 positive staining was found with disease progression as summarized the results in Table 3. The positive TR4 staining was 0.89% (1/111) in benign tissue cores (normal and BPH), 33.33% (9/27) in PIN cores, 76.3% (68/89) in LG and 86.7% (58/67) in HG. To cover the full spectrum of cancer progression, we examined TR4 expression in the invasive prostate cancer specimens that metastasize to bone. A total of 10 bone metastasis prostate specimens were examined, 60% were scored as positive while 40% were negative, only one sample stained positive for nuclear TR4. Interestingly, the percentage of cytoplasm is increased with the disease progression (FIG. 19) where none of cytoplasma TR4 was found in normal, 22.2% (4/18) in PIN, 57.1% (36/63) in LG, and 70% (41/58) in HG (Table 3). TR4, as a transcriptional factor, moved to nucleus when responding to stress, the increasing of cytoplasm retention of TR4 prostate carcinoma indicating a dysregulation of TR4 in the prostate carcinoma which contributes to the tumor progression.

TABLE 3
Quantification of TR4 staining and in prostate TMA analyses

| | Postive | | | |
|---|---|---|---|---|
| | Postive/Negative = Total | Negative | Total | % |
| Normal | 0/1 = 1 | 43 | 44 | 2.27% |
| BPH | 0/0 = 0 | 68 | 68 | 0% |
| Benign | 0/1 = 1 | 111 | 112 | 0.89%^{c,d} |
| PIN | 4/5 = 9 (44%)^{a} | 18 | 27 | 33.3%^{c,e} |
| LG | 36/32 = 68 (52%)^{a} | 21 | 89 | 76.3%^{f} |
| HG | 41/19 = 58^{b} (702%)^{a} | 9 | 67 | 86.7%^{f} |
| Cancer | 77/51 = 126 | 30 | 156 | 80.7%^{d,e} |
| Meta | 6/1 = 6^{a} | 4 | 10 | 60% |

^{a}% of cytoplasm staining;
^{b}two HG and on emeta show both cytoplasm and nucleus staining.
A significant difference between benign and PIN (^{c}p < 0.00001);
benign and cancer (^{d}p < 0.00001)
PIN and cancer (^{e}p < 0.0005);
LG and HG (^{f}p < 0.05)

h) Research Design and Methods:

(1) Examination of the Signal Pathways Involved in TR4 Mediating Cellular Senescence, as Well as to Determine Effects of TR4 Deficient-Induced Senescence-Associated Change and their Influence on Epithelial Cell Growth and Transformation.

Senescence, defined as permanent and irreversible proliferation arrest, is a cellular defensive response to stresses including telomere shortening, DNA damage, oxidative stress and oncogene activation. It has been proven that senescence is an initial barrier in cancer development in some recent studies, which consistently revealed the occurrences of senescence in different types of premalignant tissues in human and mouse. TR4 KO mice have a shortened lifespan and display features of premature aging. TR4 KO MEFs, which have higher endogenous ROS, developed a rapid replicative senescence compared with wt MEFs. Therefore, loss of TR4 results in higher oxidative stress and genomic instability, which triggers senescence as the cellular defense system against tumorgenesis. In human, most of the age-related cancers arise from epithelial cells, such as breast, colon, and prostate. How aging affects the cancer pathways, is still largely unknown. Early reports found that senescent fibroblasts can promote epithelial cell growth and tumorigenesis supporting that microenvironment provided by stroma is an active contributor to tumor growth (Parrinello S, et al. 2005 J Cell Sci 118:485-96; Campisi J 2005 Cell 120:513-22). Herein the signal pathways involved in TR4 mediating cellular senescence are elucidated, with focus on two pathways (DePinho RA 2000 Nature 408:248-54) Gadd45α, a potential TR4 target gene, and (Sharpless N E, DePinho RA 2005 Nature 436:636-7) p53-dependent pathways. To investigate if accelerated aging of TR4 KO fibroblasts can promote pre-malignant and malignant epithelial growth, fibroblasts from TR4 KO are examined at pre-senescence (p2-3) and senescence stages (after P4), compared with TR4 wt fibroblasts at the same stages, can stimulate pre-malignant/malignant epithelial cells growth and transformation. Herein disclosed are insights into the roles of TR4 plays in this anti-cancer barrier-senescence defense networks as well as the determination if cells can be bypassed during oncogenic transformation if TR4 is altered.

(2) Confirmation of TR4 Role in Cellular Senescence:

Senescence is a permanent and irreversible growth arrest in cells as a response to various stresses. Recent studies have linked senescence to aging and tumorigenesis. Data found that TR4 KO MEFs stopped proliferating and showed flat vacuolated morphology typical of senescent cells as early as P4 with G2/M arrest, while normal MEFs grew well for approximately 6-8 population doublings before proliferation began to decline, and after 12-15 doublings, the culture senesced. Human TR4 RNAi are transfected into WI-38 cells, then selected with puromycin, and the clones containing TR4 RNAi are further characterized for the cell morphology, growth rate, ROS response, DNA damage, senescence-associated-β-galactosidase staining, and cell cycle profiles analyses. Cell senescence genes, such as p53, p16, p21, Rb are compared.

(a) Cell Proliferation Assay:

Cell proliferation rate are determined by 3H-thymidine incorporation analysis and MTT assays. For 3H-thymidine incorporation analysis, cells are incubated for 24 h with medium containing 0.25 μCi/ml 3H-thymidine. The radioactivity incorporated is measured by liquid scintillation counting. For MTT assay, the conversion of a colorless substrate to reduced tetrazolium by the mitochondrial dehydrogenase, are used to assess cell viability and growth. After each treatment period, 10% volume of medium of thiazolyl blue (5 mg/ml, Sigma) is added into each well for 2-3 h at 37° c. The resultant precipitate are dissolved in 0.04 M HCl in isopropanol and absorbency are read at a wavelength of 570 nm with background wavelength at 660 nm.

(b) Retroviral-Mediated Gene Transfer:

To over-express and knockdown TR4 expression in WI38, pBabe-hTR4, and pRetro-TR4 RNAi are used for retroviral infection. Ecotropic packaging cells are plated for 24 h and then transfected with SuperFect (Quigen) with pBabe-pur/2 or pBabe-TR4 (for overexpression), or pRetro-scramble, or pRetro-TR4 RNAi (for knockdown). After 48 h the viral containing medium are filtered (0.45 mM filter, Millipore) to obtain viral-containing supernatants. Targeted MEF cells are plated and the culture medium is replaced with a mix of the viral-containing supernatant and culture medium, supplemented with 4 μg/ml polybrene, and the cells are incubated at 370 C. WI38 cells infected with the empty vector (pBabe-puro) and scramble RNAi are used as controls. The TR4 RNA level are determined by Q-PCR, and confirmed by Western Blotting analysis.

(c) Senescence-Associated β-Galactosidase Staining:

Cells are washed with PBS and fixed for 5 min in 2% formaldehyde and 0.2% glutaraldehyde. Fixed cells are washed with PBS and incubated with fresh senescence-associated β-galactosidase staining solution (sodium phosphate buffer, pH 6.0 containing 1 mg of X-gal/ml 40 mM citric acid, 5 mM potassium ferrocyanide, 5 mM potassium ferricyanide, 150 mM NaCl, 2 mM MgCl2). Staining is detected by light microscopy following overnight incubation.

(d) Determination of TR4 Expression by Real-Time PCR (Q-PCR) and Western Blotting Analyses.

WI 38 cells stably transfected with pBabe-TR4 or pRetro-TR4RNAi are harvested. The RNA samples are obtained by Trizol reagents, and total RNA are converted into first strand cDNA by SuperScript III reverse transcriptase (Invitrogen). Primers for amplification of TR4 are designed by the Becon Primer Designs software. Q-PCR is performed using Bio-Rad iQ cycler. CT values are calculated and normalized to the level of the housekeeping gene α-microglobulin. Relative gene expression are calculated according to 2-ΔΔCT from three independent experiments. To confirm the expression changes in protein level, cells are lysed by RIPA buffer and quantified. Proteins are separated by 12% SDS-PAGE and blotted with anti-TR4 antibody (#15 monoclonal antibody) to detect TR4 expression.

(3) Exploration of the Senescence Pathways Mediated by TR4 Deficiency:

Senescence is a complex, molecularly heterogeneous cellular protective program which could be triggered by different intrinsic or extrinsic stresses through different pathways in different tissues and cell types. The exploration of the molecular mechanism for TR4 deficiency induced senescence is clearly necessary to understand senescence as an anticancer mechanism as well as TR4's role as a caretaker. Herein it was found that TR4 can protect cells from the oxidative DNA damage-induced cellular decay, at least partially via the modulation of Gadd45a gene activity. Disruption of Gadd45a in mice results in genomic instability and increased carcinogenesis, therefore, Gadd45a is an important component in the cellular defense network that is require for maintenance of genomic stability. Herein disclosed are methods to show whether TR4 deficiency-mediated cell senescence is Gadd45a dependent.

Therefore, restoring Gadd45a into TR4 KO cells, and blocking of Gadd45a in wt MEFs are performed. Senescence can constrain cells oncogenesis by many mechanisms, including telomere attrition and induced tumor suppressor genes, and all conditions activate p53. Whether activation of p53 and its downstream pathway represent the major force that leads to TR4 KO MEFs senescence are further examined. The involvement of TR4 deficiency-mediated senescence in the activation of p53 and/or Rb is determined. TR4 KO vs wt MEFs are challenged with genotoxic stresses, including UV, IR, and H₂O₂, and the p53-mediated signal pathways in response to stresses are examined.

(a) Confirmation of TR Deficiency-Mediated Cell Senescence Via the Loss of Gadd45a.

Herein it is disclosed that Gadd45a is significantly reduced in TR4 KO, and TR4 regulates Gadd45a activity by binding to DR3 in the Gadd45 intron 3. It is disclosed herein that TR4 can protect cells from DNA-damage through at least, partial mediated up-regulation of Gadd45a, while loss of Gadd45a in TR4 KO leads to cellular senescence. For further confirmation, endogenous Gadd45a are blocked by RNAi to test if the cells can lose their TR4 protective effects, leading to an early onset of cellular senescence. MEFs from wt cells are stably transfected with Gadd45a RNAi (pSuperior vector) and scrambled RNAi control and then test their response to genotoxic challenge. Meanwhile, whether the cellular senescence in TR4 KO can be delayed by restoring Gadd45a in TR4 KO cells by expression of pBabe-Gadd45a via a retro-viral delivery system is tested.

(b) Determination Whether the Activation of p53 is Involved in TR4-Deficiency-Mediated Cell Senescence.

TR4 KO MEFs display an early onset of cellular senescence possibly overloaded oxidative stresses and DNA damages. Accumulated information has demonstrated that cellular senescence induced by DNA damage, oxidative stress, and activation of oncogenes is mainly activated by the p53 pathways through the ATM/ATR, or through p14/ARF protein. p21, a p53 target can then cause Rb activation by inhibiting CDK2/cyclin E activity. Most cellular stresses activate the P16/INK4a gene as well, which also leads to Rb activation through the inhibition of CDK4/CDK6 activities. Although the master regulators of senescence are p53 and Rb, a variety of other genes, like MAPK cascade, may function in this machinery. The MEFs from TR4 KO are challenged at pre-senescence stage (p1-3), and senescence stage (after P4) with H₂O₂ (200 μM) for 2 h, (0, 3, 6, 9, 12, and 15 Gys), and to UV (0, 1, 2, 4, 8, 16 J/m²) and harvest the cells at day 1, 3, and 5. Cell survival rate are determined by cell growth and proliferation rate using ³H-thymidine incorporation and MTT assays. The mRNA and protein extracts from cells in each step, are harvested, and the p53 pathways, including ATM, ATR, p21, p53, Rb, p19, p14/ARF, and cyclin E status (total protein and phosphorylation status) are examined. Q-PCR are used to quantify the changes in RNA levels, and Western blotting are used to further confirm the expression and phosphorylation status of those p53 pathway related proteins.

(c) Detailed Methods:

(i) Construction of pBabe-Gadd45a and pRetro-HiG-Gadd45RNAis.

wt MEFs/tissues are used to clone Gadd45α gene (BC 011141). Total RNA are isolated and Gadd45a cDNA are generated by RT-PCR by pairs of primers that covered the full length of mouse Gadd45a cDNA. RNAi are designed according to the Block-iT RNAi Designer (Invitrogen) and knockdown efficiency are tested by Q-PCR, and then further confirm by Western Blotting.

(ii) Cell Proliferation, Cell Cycle Profile, Senescence-Associated β-Galactosidase Staining, Q-PCR, and Western Blotting Analyses:

Cells, including MEFs or WI38 with different expression levels of TR4, are harvested after exposure to genotoxic stresses. Protein and RNA are extracted and analyzed. The methods above follow the protocols described previously.

(4) Determine Effects of TR4 Deficiency-Induced Senescence-Associated Change and their Influence on Epithelial Cell Growth and Transformed.

In human, most of the age-related cancers arise from epithelial cells, such as breast, colon, and prostate. Factors, like mutations, stroma, and microenvironment of tissue are particularly important for the initiation and progression of epithelial cancers. How aging affects these factors to influence cancerous pathways, are still largely unknown. Early reports found that senescent fibroblasts can promote epithelial cell growth and tumorigenesis supporting that microenvironment provided by stroma is an active contributor to tumor growth. It was found herein that the TR4 KO fibroblasts display an early onset of cellular senescence due to higher level of cellular ROS and DNA damages. It is disclosed herein that accelerated aged fibroblasts derived from TR4 KO provides a geriatric microenvironment that permits or promotes pre-malignant and malignant epithelial growth. Thus, whether fibroblasts from TR4 KO at pre-senescence (P2-3) and senescence stages (after P4), compared with TR4 wt fibroblasts at the same stages, can stimulate pre-malignant/malignant epithelial cells growth, and transformation is examined. The goal is to confirm the linkage of cancer pathways with cellular senescence induced by TR4 deficiency.

(a) Examination of Effects on the Growth of Prostate and Breast Epithelial Cells by Senescent Stroma from TR4 Deficient Fibroblasts In Vitro and In Vivo

In order to determine whether TR4 deficiency-induced aging stroma can alter the cellular microenvironment to stimulate the neighbor cells-epithelial cells growth, and facilitate the progression of epithelial malignancies, TR4 KO senescent MEF are used to test for their ability to induce epithelial-original cell growth and transformation by co-culture system. TR4 KO senescent stroma stimulation of growth on prostate epithelial cells is examined. Meanwhile, testing is applied to the breast epithelial cells, which Campisi's group (Krtolica A, et al. 2001 Proc Natl Acad Sci USA 98:12072-7) has established, as controls. Therefore, two types of epithelial cells, prostate and breast, from both pre-malignant (immortalized yet susceptible to transformation) and malignant stages are examined. In addition to using TR4 KO MEFs, TR4 RNAi technique is applied to knockdown TR4 in human fibroblast WI 38 to induce rapid cellular replicative senescence, and then tested their effects on epithelial cells growth. Cell proliferation, in vitro tumorigenesis assays is performed. The epithelial cells which show stroma-induced cell transformed in vitro, are tested further in vivo xenograft nude mice model. Table 4 summarizes the fibroblast and epithelial cells that are used to test the stroma-epithelia interaction.

STROMA

| | | |
|---|---|---|
| Mice | pre-senecsence | TR4 wt MEF (p2-4) |
| | | TR4KO MEF (p2) |
| | senescnece | TR4KO MEF (>p4) |
| Human | pre-senescence | WI38 |
| | senecence | WI38-TR4RNAi |

EPITHELIAL

| | |
|---|---|
| PROSTATE | BREAST |

non-tumorigenic

| | |
|---|---|
| BPH-1 | S1 |
| RWP-1 | 184B5 |

tumorigenic

| | |
|---|---|
| LNCaP | MDA231 |

(b) Cell Co-Culture System:

A contacted co-culture system is established to study the interaction between epithelial and stroma cell to the cell growth and transformation. The contacted co-culture system that measures the cell proliferation follows the methods described by Campisi's group (Krtolica A, et al. 2001 Proc Natl Acad Sci USA 98:12072-7). Briefly, the prostate/mammary epithelial cells including immortalized but non-tumorigenic: BHP, RWP-1, S1, and 184B5; cancer: LNCaP, MDA231 are co-cultured with pres-senescent (cells contain >70% proliferating) and senescent (cells contain <10% proliferating) stroma cells. Stroma cells are cultured first and allowed to attach to 6-well culture dishes overnight and then change to serum-free medium for 1-3 days to generate lawns. Epithelial cells are incubated with growth-factor deficient medium for 2-3 days, plated on the fibroblast lawns for 8 days. Cultures are fixed in 4% paraformaldehyde and are stained with 1% Rhodanile blue or (1 μg/ml) DAPI. Fluorescent images from five random filed/wells are analyzed. DAPT stains the nuclei, and the epithelial fluorescence/filed is determined by distinguishing epithelial (smaller, more intense) and fibroblast (large and less intense) cells.

(c) Invasion Assay:

LNCaP, PC-3 and DU 145 cells are seeded and cultured for 72 h in regular medium. Cells are harvested and counted, and 5×10⁴ cells/chamber are used for each invasion assay. Cells are added to Matrigel coated inserts (Becton Dickinson Labware, Bedford, Mass.) in normal medium. The lower chambers contained the conditioned medium from pre-senescent and senescent TR4 stroma fibroblast cells. The chambers are incubated for 22 h at 37° C. The cells that invade to the lower surface of the membranes are fixed and stained with 1% Toluidine blue, and five random fields are counted under light microscope.

(d) Tumorigenicity (Colony Forming Assay):

Anchorage-dependent and -independent colony forming assays are applied to characterize the tumorigenicity of cells. In anchorage dependent colony forming assays, cells (epithelial alone, or with stimulation from stroma co-culture cells) are seeded at a density of 200 cells/100 mm dish. Medium are refreshed twice per week for three weeks. The plates are stained with crystal violet in methanol, and colonies containing more than 50 cells are counted. In anchorage independent colony forming assays, treated cells are suspended at a density of 2000 cells/ml in 0.4% low melting point agarose in 10% FBS/RPMI, and plated on top of 1 ml underlayer of 0.8% agarose in the same medium, in 6-well culture plates. Cultured cells are fed twice per week, and large colonies are stained with p-iodonitrotetrazolium violet and counted after three weeks.

(e) Nude Mice Xerograph Model:

Nude male mice (for prostate) and female mice (for breast), are maintained for 2-4 weeks prior to the tumor studies, and housed under normal lighting. Young nude mice (6-8 weeks old) are injected (100 μl) subcutaneously into the dorsal flap with 2−3×106 prostate epithelial cells alone, or with pre-senescent and senescent fibroblasts at 2−3×10⁶, or into the nipple region with 2−3×10⁶ breast epithelial cells alone or with pre-senescent and senescent fibroblasts at 2−3×106. Tumors are allowed to grow, measured three times every week with calipers, and tumor volumes are calculated using the formula 0.532×r12×r2 (r1<r2). In all animals, once it is observed that increased tumor volumes reach into 10% of body weight, animals are sacrificed; otherwise animals are sacrificed at the end of 12-week after cells implantation. Tumor-bearing animals from all groups are sacrificed by cervical dislocation and blood is collected. Tumors are excised, weighed, and half of the tumor is stored in liquid nitrogen for later analysis. The other half of the tumor are fixed and embedded for immunohistochemical analysis. The prostate gland/mammary gland, lung, lymph nodes, and bone marrow are examined for tumor metastases. Ten animals per group are analyzed.

(f) Determine the Senescence-Induced Factors/Pathways Contributing to the Altering of Cellular Microenvironment Leading to the Cell Growth and Transformation.

Because Gadd45α mRNA was lower in TR4 KO, a determination can be made as to whether Gadd45a is the driving force that triggers early senescence found in MEFs and contributes to the changing microenvironment stimulating epithelial cell proliferation by expressing Gadd45a in TR4 KO MEFs. The identified pathways/factors that are altered and responsible for cellular senescence in TR4 MEF are tested. Those factors are “restored” back to the TR4 KO MEFs via re-expression of those factors, if they are less in TR4 KO; or the factors are “knocked-down,” if there are more in TR4 KO. The modulation of those factors in TR4 KO can reverse the TR4 KO defects, and interfere or prevent prostate epithelial cell growth and transformation.

(5) Elucidation of the TR4 Roles in Controlling the Cell Senescence vs Cellular Oncogenic Pathways In Vitro and in Vivo.

Cellular senescence is a response to stress, such as DNA damage, oxidative stress, oncogenic activity, and plays an important role in tumor suppression and contributes to organism aging (Campisi J 2005 Cell 120:513-22; Neumeister P, et al. 2002 Int J Biochem Cell Biol 34:1475-90). Lack of TR4 in mice results in accelerated aging, and early onset of premature cellular senescence strongly supports the TR4 role in guarding genome stability via both reducing oxidative stress and promoting DNA repair capacity. It has become clear that loss of genome stability due to malfunctioning DNA repair machineries can have catastrophic consequences, such as cancers and premature aging. The studies identify TR4 as a key regulator in the ROS-DNA damage-DNA repair cascade that not only links TR4 to these genomic maintaining networks, but also suggest that TR4 participates directly in making life and death decisions in the cells. It is disclosed herein that TR4 is a caretaker gene which protects the genome from mutations; and lacking TR4 create a pro-oncogenic tissue environment to synergize with activation of oncogene. Tumorigenic conversion of primary fibroblasts requires at least two cooperating oncogenes, or in combination of inactivation of tumor suppressor genes (Weinberg RA 1983 J Cell Biol 97:1661-2; Land H, et al. 1983 Nature 304:596-602; Ben-Porath I, Weinberg RA 2005 Int J Biochem Cell Biol 37:961-76). To gain further insight into the mechanisms of TR4 effects on the cellular response to oncogenic stress, the cellular response to Ras-oncogenic insults is examined by altering TR4 amount in the cells using knockdown (TR4 KO or TR4 RNAi) and overexpression of TR4 in SV40LT immortalized MEFs cells. TR4 anti-ROS and anti-DNA damage effects are examined in the human fibroblast cells WI-38 and WI-38 immortalized with SV40-TAg in vitro and in vivo. This shows the TR4 roles in guarding genomic integrity (via sensing stress, anti-ROS, and DNA repair) by introducing Ras to the cells, which induces cell growth arrest or cell transformation and also show that TR4 can alter the cell fate by preventing DNA damages.

(6) Examine the Effects of TR4 on Cellular Senescence in Human Fibroblast Cells.

Loss of TR4 shortened the MEFs lifespan in which most of the cells arrest in G2/M, supporting the roles of TR4 in guarding the genomic integrity. Cellular senescence is a program executed by cells in response to a variety of stresses, including DNA damage, oxidative stress, oncogene activity, and others (Serrano M, et al. 1997 Cell 88:593-602; Ben-Porath I, Weinberg RA 2005 Int J Biochem Cell Biol 37:961-76; Rangarajan A, et al. 2004 Cancer Cell 6:171-83). Laboratory mice have represented a powerful experimental system for understanding the intricacy of human cancer pathogenesis. Indeed, much of the current conceptualization of how tumorigenesis occurs in humans is strongly influenced by mouse models of cancer development. However, an emerging body of evidence indicates that there are fundamental differences in how the process of tumorigenesis occurs between mice and humans. Human TR4 RNAi are transfected into WI-38 cells, then selected with puromycin, and the clones containing TR4 RNAi are further characterized for the cell morphology, growth rate, ROS response, DNA damage, senescence-associated-β-gal staining, and cell cycle profiles analyses. Cell senescent genes, such as p53, p16, p21, and Rb are compared.

(7) Determine the Roles of TR4 on the Ras-Mediated Cell Transformation in the Immortalized MEFs In Vitro and in Vivo.

It has been demonstrated that perturbation of two signaling pathways involving p53 and Ras suffice for the tumorigenic conversion of normal murine fibroblasts. As indicated herein, TR4 serves as a guard to protect the cells from genotoxic stress. Thus, TR4 can prevent the cells from Ras-mediated oncogenic pathway in SV40LT immortalized MEFs with inactivated p53. It is disclosed herein that TR4 not only protects cells from ROS-induced DNA damage-mediated cell senescence, but also prevents cells against cellular tumorigenic transformation. MEFs from wt and TR4 KO are immortalized by SV40LT and then a retroviral vector encoding Ras oncogene (pWZL hygro-H-RasV12) and vector control are introduced to induce cell transformation. The immortalized MEFs from TR4 KO and wt are examined for their cell transformation capacity judged by cell morphology, cell growth, DNA damage degree, cell cycle profile, and soft agar assays. Finally, the MEFs/human fibroblasts showing anchorage-independent growth in soft agar are used to confirm tumorigenicity in nude mice xenografts.

(8) Determine the Roles of TR4 on the Ras-Mediated Cell Senescence Vs Cell Transformation in the Immortalized Human Fibroblast In Vitro and In Vivo.

Expression of multiple oncogenes and inactivation of tumor suppressors is required to transform primary mammalian cells into cancer cells. Recent evidence indicates that human cells require more genetic changes for cell transformation than do their murine 10 counterparts. Activation of Ras is usually associated with cancer; however, it produces premature senescence in primary immortalized human fibroblasts. Herein, the ability of the modulation of TR4 expression in these Ras-activated immortalized human fibroblasts (WI-38 VA-13, ATCC) to bypass Ras-induced senescence and allow Ras to transform these cells is tested. Stable cells with different degrees of TR4 expression levels (normal, knockdown, and overexpression) are examined to determine the cell characteristics. Cells that grow in soft agar, are further tested for their in vivo tumorigenicity in nude mice xenografts.

(a) Detailed Method

(i) Generation of MEFs.

Matings are set up by using TR4 heterozygous males and females, and checking for vaginal plugs every early morning. Once obtaining an E14.5 female, the mouse are sacrificed and dipped in 70% ethanol. Embryos are collected, and placed in a sterile Petri dish with PBS, remove heads and all the internal organs (liver, heart, kidney, lung, and intestine) from embryos and after that wash with PBS twice. Place the tissues in ˜5 ml of DMEM culture medium, pass it through a 22 gauge needle a few times. Transfer the minced tissue into a 25 cm2 tissue culture flask which contains 10 ml medium, and culture overnight at 37° C. (5% CO2). Change the medium after 25 h to remove unattached cells and debris. After 2 or 3 days of culture the MEF cells form a confluent monolayer; then trypsinize each plate and split them 1/5. All the experiments are finished before P4.

(ii) Retroviral-Mediated Gene Transfer:

To restore TR4 expression in TR4 KO MEFs, pBabe-hTR4 are used for retroviral infection. Ecotropic packaging cells are plated for 24 h and then transfected with SuperFect (Quigen) with pBabe-pur/2 or pBabe-TR4. After 48 h the viral containing medium are filtered (0.45 mM filter, Millipore) to obtain viral-containing supernatant. Targeted MEF cells are plated and the culture medium replaced with a mix of the viral-containing supernatant and culture medium, supplemented with 4 μg/ml polybrene, and the cells are incubated at 370 C. MEF cells infected with the empty vector (pBabe-puro) are used as control.

(iii) Immortalization of MEFs:

Once MEFs cells are generated, they are immortalized by expressing SV40LT in the MEFs. Viral vector encoding SV40LT cDNA (pBabe-SV40LT) are transduced with retrovirus and purified with puromycin (1-2 μg/ml) for 2-4 days, and the changed into cultured medium. The survival cells are tested for the expression of LT Ag.

(iv) Ras Oncogene Overexpression.

The immortalized MEFs and human fibroblast WI-38 VA-13 cells are infected with retrovirus containing Ras oncogene (pWZL hygro-H-RasV12). To eliminate the uninfected population, cells are selected with hygromycin B (100 μg/ml) for 2-4 days. After selection is complete, cells are examined for expression/activity of Ras and its down-stream activators, such as Raf, MEKK, and ERK. The cells then are tested for the tumorigenicity by anchorage-independent growth assay.

(v) Senescence-Associated β-Galactosidase Staining:

Cells are washed with PBS and fixed for 5 min in 2% formaldehyde and 0.2% glutaraldehyde. Fixed cells are washed with PBS and incubated with fresh senescence-associated β-galactosidase stain solution (sodium phosphate buffer, pH 6.0 containing 1 mg of X-gal/ml 40 mM citric acid, 5 mM potassium ferrocyanide, 5 mM potassium ferricyanide, 150 mM NaCl, 2 mM MgCl2). Staining is detected by light microscopy following overnight incubation.

(vi) Anchorage-Independent Growth Assay:

Soft agar assays are performed as described previously. Briefly, individual cell lines are seeded in triplicate at three different dilutions ranging between 1×10³ and 5×10⁵. Each experiment are repeated at least once. Colonies are counted and photographed between 18-24 days under phase contrast microscopy.

(vii) Tumorigenicity Assay:

6-8 week old athymic nude mice are injected subcutaneously, bilaterally into dorsal-lateral flanks with 0.2 mixture of 2×10⁶ cells mixed with Matrigel (v:v=1:1). The growth of tumors is monitored by measuring the tumor volume. Tumor volume is calculated as v=L×12×0.52, where L and 1 represent the larger and the smaller tumor diameter, respectively, measured daily.

(9) Examination of TR4 Expression Status in Prostate Cancers by Tissue Array Analysis, and Determination of TR4 Roles in Mediating Cancer Progression.

Aging is one of the major risk factors for prostate cancer, therefore, examination of TR4 expression in normal prostate vs. prostate cancer can provide information of how TR4, as a caretaker, is involved in cancer development. TMA analysis of TR4 protein abundance in clinical prostate specimens reveals numerous abnormalities. First, TR4 expression was significantly increased in PIN, LG and HG tumors as compared to normal prostate specimens, in which TR4 is expressed mainly in the nucleus of basal cells (FIG. 17). Interestingly, in contrast to nuclear basal cell staining of TR4 in normal prostate, a shifted TR4 staining from nucleus to cytoplasm was increased proportionately to the degree of disease. TR4, as a transcriptional factor, moved into the nucleus when responding to stress (FIG. 14), the increase of cytoplasm retention of TR4 prostate carcinoma indicating a dysregulation of TR4 in the prostate carcinoma. These results indicate TR4 becomes activated in the early stages of tumorigenesis and a shifted TR4 staining from nucleus to cytoplasm during tumor progression indicates that caretaking functions/activities of TR4 are dependent on its nuclear import/export. It is possible that cancer cells escape from TR4 protective effects by inactivation of TR4 through either mutation or other modulations on TR4 activity, which retains TR4 in the cytoplasm. Similar to a recent paper showing that DNA damage response is an anti-cancer barrier which is activated in early tumorigenesis to delay/prevent cancer, and mutations compromising this checkpoint increases genomic instability and tumor progression (Bartkova J, et al. 2005 Nature 434:864-70). Whether TR4 is mutated in prostate cancer tissues in which TR4 is inactivated due to its cytoplasmic retention is determined as well as whether such TR4 mutation(s) precedes the mutations or loss of p53 that are found almost exclusively in advanced prostate cancer. Furthermore, how TR4 mRNA expression, protein expression/stability, and nuclear transport are regulated in response to genomic and cellular stresses, such as UV, ionic irradiation (IR), and reactive oxygen species (ROS) is investigated.

(10) Determination of TR4 and p53 Status by the Prostate Cancer TV Analysis.

The tissue microarray analysis (TMA) is an advancement that can analyze multiple different tissue samples on one slide, and subject to joint analysis by immunohistochemical (IHC) staining. The abnormal expression patterns of TR4 in the prostate cancer is tempting for an examination to determine if this “dysfunction” of TR4 precedes the inactivation of p53, the most predominant tumor suppressor gene which is found lost or mutated almost exclusively in advanced prostate cancer. Also, TR4 function is examined in prostate cancer by investigating Gadd45a, a target gene of TR4. The correlation between TR4, p53, and Gadd45 with the progression of prostate cancer reveals the roles of TR4 in this DNA-damage-repair network and its contribution to the tumor progression, if TR4 dysfunction. TMA analyses on p53 (total and Ser-15-phosphorylated p53), Gadd45 and TR4 is continued and then their expression density and expression pattern is correlated with prostate cancer progression status. In addition, TR4 IHC is performed on the invasive prostate cancer specimens in continue collaboration with Department of Pathology. The methods disclosed herein provide the status of TR4, its correlation with p53, and down-stream targets, which can lead to a better diagnosis of prostate cancer progression and better prediction of clinical outcomes.

(a) Detailed Methods:

(i) Prostate Cancer Tissue Microarray:

Prostatic adenocarcinoma cases over a 2-year period were reviewed at the University of Rochester Medical Center-Strong Memorial Hospital and eighty cases were selected. Areas for sampling were designated as normal (N), hyperplastic (BPH), high-grade prostatic intraepithelial neoplasia (PIN), low-grade adenocarcinoma (LG), and high-grade adenocarcinoma (HG). Tumors were classified as follows: Gleason pattern 1, 2 and 3 were labeled low-grade and Gleason pattern 4 and 5 were labeled high-grade. A total of 50 N, 82 BPH, 35 HGPIN, 104 LG and 82 HG areas were chosen for sampling, averaging 4-6 cores per case. Each core are examined under a light microscope and separately scored. Cores that had less than 50% of original tissue present are disregarded.

(ii) Immunostaining of TR4/p53/Gadd45a:

Immunocytochemical stains using a monoclonal antibody to TR4 #15 (Lee Y F, et al. 1998 J Biol Chem 273:13437-43; Lee Y F, et al. 1999 J Biol Chem 274:16198-205) at 1-400 dilution are performed on tissue microarray sections constructed as described above. For p53, immunohistochemistry is a major method of investigation p53, based on the observation that mutant p53 protein is frequently stabilized, antibodies against p53 (Chen Z, et al. 2005 Nature 436:725-30) (rabbit polyclonal anti-p53, CM5; Novocastra) are used. In addition, the p53 phosphorylation as a critical component of activation of p53, the Ser-15-phosphorylated p53 (Bartkova J, et al. 2005 Nature 434:864-70) (Calbiochem) are applied. The dilution fold for antibodies is tested to optimize the staining conditions before performing TMA. For Gadd45α IHC, the rabbit anti-human polyclonal GADD45α, antibody (200 μg/ml, Santa Cruz Biotechnology) is used. Paraffin tissue blocks are cut at 4 to 5 microns and floated on distilled water at a temperature of 52° C. Sections are mounted on chemically charged slides followed by room temperature drying until opaque then are placed in the oven at 58-60° C. overnight. Sections are deparaffinized according to established procedures. Sections are quenched with 3% hydrogen peroxide for 6 minutes, then cleared in running water followed by TBS (50 mM Tris-HCL, 150 mM NaCl, 0.05% Tween 20 at pH 7.6). Antigen unmasking was performed by the following method: Slides are heat treated with Dako antigen retrieval solution (Citrate Buffer pH 6.1) in a Biocare Medical Decloaking Chamber for 12 minutes at 120° C. The slides are then rinsed with Tris Buffered Saline (TBS) for 5 minutes. Sections are stained for 60-minutes at the specified titer. Follow by 30-minute incubations in both Goat Anti-Rabbit IgG-Biotin (Vector Laboratories, Inc. Burlingame, Calif.) and Streptavidin-HRP. All slides are developed with AEC+ (Dako) for 10 mins. Modified Mayer's Hematoxylin are used as a counterstain and slides are blued in 0.3% ammonia water followed by a tap water rinse. Cover slips are mounted using an aqueous media.

(iii) Scoring and Statistic Analyses:

The slides are examined under 20× power with a light microscope. Cores with less than 50% of the tissue of interest remaining after processing are disregarded. Staining intensity are recorded as a range from 0 to 3 (0=no, 1=weak, 2=moderate, 3=strong staining), compared to the control tissue staining. The IHC score are derived by multiplying staining intensity (0-3) by the percentage of cells stained resulting in a product between 0-300. The mean score from each group of related tissue cores (i.e. cores from the same case) are used to provide a final IHC score. A cutoff IHC score of 100 are used to determine positive versus negative results (Kreisberg J I, et al. 2004 Cancer Res 64:5232-6). The results are then tabulated and tested statistically using Fisher Exact test. The nucleus vs cytoplasmic staining is recorded.

(11) To Determine the Molecular Status of TR4 and its Relationship and Genomic (in)Stability in Human Prostate Cancer.

Disclosed herein, TR4 is involved directly in DNA damage repair networks to promote DNA repair and maintain genome stability. TMA analysis reveals an over-expressed and abnormal increasing cytoplasmic stainings of TR4 in tumor stages. As a transcriptional factor, TR4 moves into the nucleus when cells were treated with genotoxic stress such as H₂O₂. Thus, TR4, as a caretaker, can be up-regulated in response to the genomic instability in the early tumor development stage such as PIN; however, cancer escapes from TR4 protective effect, where most of TR4 is retain in the cytoplasm and non-functional. Therefore whether TR4 is mutated, especially in the nuclear translocation signals, in prostate cancers in which TR4 is inactivated and retained in the cytoplasm, and/or TR4 is associated with other cytoplasmic factors that prevent TR4 from getting into the nucleus is determined. A determination is made as to whether such TR4 mutation(s) precede mutations or loss of p53 that are found almost exclusively in advanced prostate cancer. TR4 is isolated from prostate cancer vs normal from a urological tissue bank. More than 250 cases of prostate cancer samples were collected for the past several years. First, TR4 is cloned from 10 tumor samples, in which more than 90% tissue population are identified as tumor by the histological analysis. The normal prostate tissue is a control. Meanwhile, TR4 from prostate epithelial cells, like BPH-1, RWP1, and prostate cancer cells, LNCaP, PC-3, DU145, and CWR22rv-1 is cloned and sequenced. In addition, genome-wide SNP arrays are performed on these prostate tissues/cells to determine whether there is loss of homozygosity in the TR4 locus and other loci. To correlate with p53 status, p53 from those tissues and cell lines is cloned and sequenced. From TR4 and p53 sequencing and SNPs data analysis, it is determined (a) whether there is TR4 mutation(s) in prostate cancer, (b) whether TR4 mutation(s) precedes p53 mutation(s) and (c) whether mutation(s) of TR4 correlates with progression of tumors.

(i) Cloning and Sequencing

TR4 from normal vs prostate tumor tissue and cells. Total RNA are isolated and cDNA are generated by RT-PCR. cDNA sequences of TR4 are determined by automatic DNA sequencing. To correlate TR4 with p53 status in the analysis samples, p53 is cloned and sequenced.

(ii) Single-Nucleotide Polymorphisms Assay:

Genome-wide SNP arrays are performed on the Affymetrix GeneChip Human Mapping 10K Array from these tissues to determine whether loss of homozygosity in the TR4 locus and other loci. These arrays contain 10,204 unique SNPs with the median physical distance between adjacent SNPs of 105 kilobases. Heterozygosity for the array averages 0.37. Each SNP is represented by sense and anti-sense oligonucleotides for each SNP variant as well as single basepair mismatches. Genomic DNA are extracted from each cell line/tissue using Qiagen Qiamp DNA Mini Kit according to the manufacturers directions. Genomic DNA are digested with XbaI and then ligated to adapter oligonucleotides, which serve as priming sites for sequence independent PCR amplification. For the quality control, an aliquot of the PCR reaction are analyzed by gel electrophoresis. Three distinct bands from repetitive sequence DNA and a smear from single copy DNA are anticipated and observed to confirm the successful amplification of sample DNA. Following fragmentation with DNAse I and end-labeling with biotin, the DNA are hybridized to the SNP array and detected with fluorescently labeled avidin. Using Affymetrix GeneChip DNA Analysis Software each locus are assigned one of three genotypes, AA or BB homozygous, or AB heterozygous. Ambiguous values receive a “No Call” assignment.

(12) To Investigate the Molecular Mechanisms by which DNA Damage Signals Modulate TR4 Expression and Activity.

The expression of TR4 is induced by DNA-damage signals, such as IR, UV, and oxidative stress, and following by nuclear translocation of TR4, and then “activated” TR4 suppresses cellular ROS and reduces DNA damage; therefore the inactivation of TR4 results in genomic instability and leads to premature aging. All of this eventually can lead to cancer, if a mutation or activated oncogene were involved. Prostate TMA analyses reveal strong correlations between TR4 expression levels, and nuclear-cytoplasmic shifted localization of TR4 with progression of prostate cancer. Taking both in vitro and clinical data together, they indicate that TR4 is a stress-induced molecule whose expression is promoted under genotoxic stresses and thereby constrains tumor development. Deregulation of TR4 can contribute to the tumor progression. Herein, how TR4 responds to stresses, such as UV, IR, and oxidative stresses is tested, as well as, a determination made if cellular localization of TR4 contributes to TR4 activity. Stresses induce TR4 expression in multiple ways, through transcriptional, post-transcriptional regulation, translational and post-translational modification. Therefore, 5′TR4 is studied to reveal how stress-induced regulatory factors change TR4 expression, as well as to determine how TR4 is modified by stress-induced kinase cascade in response to stresses. The cellular localization of TR4 in response to stress is also studied.

(a) To Determine Whether TR4 mRNA is Up-Regulated at the Transcriptional and/or Post-Transcriptional Levels (mRNA Stability).

To test whether UV and/or ionic irradiation, and/or ROS at the level of transcription, nuclear run-on assays are performed. TR4 transcription rate are measured using nuclei from both irradiated or H₂O₂-treated cells and nonirradiated or H2O₂-treated control cells. In addition, to measuring TR4 mRNA stability, the cells are treated with transcriptional inhibitors, actinomycin or Amanitin, and quantitate TR4 mRNA expression by real-time PCR. Furthermore, TR4 gene regulation is studied by dissecting the 6 kb TR4 promoter.

(b) To Study Whether TR4 Protein is Up-Regulated at the Level of Translation and/or Post-Translation (Protein Stability).

To determine whether up-regulation of TR4 protein upon UV, IR, and H₂O₂ treatment is at the translational level, TR4 protein expression is measured after cells are treated with or without irradiation, and with a translation inhibitor, cycloheximide.

(c) To Investigate Whether TR4 Activity in DNA Repair is Modulated Through Protein Phosphorylation using TCR and NHEJ Assays and Global Genomic NER Immunoassay.

It is disclosed herein that TR4 is phosphoprotein and is dephosphorylated at least upon UV irradiation. Furthermore, TR4 contains highly stringent phosphorylation sites on Ser-144 and Ser-351 by 14-3-3, and PKCα, β, γ, and ξ. Mutation analyses showed that dephosphorylation/phosphorylation of TR4 affects transcriptional couple repair (TCR) ability (FIG. 15), therefore TR4 can be modulated by kinase/phosphotase cascades involved in cell DNA-damage repair systems. This can be shown by activating or inactivating putative kinases/phosphatase using activators or inhibitors, as well as constitutively active and dominant negative forms of kinases, and test activity of TR4 in TCR and NHEJ assays. Furthermore, in vivo and in vitro interaction and kinase assays between TR4 and putative kinases/phosphatases are performed by CoIP. When the interaction between TR4 and kinases is found, in vitro kinase assays are performed. Herein it is disclosed how the upstream stress regulates TR4 activity via phosphorylation signals, and disruption of this regulation contributes, partly, to the dysfunction of TR4 that is found in cancer.

(d) To Determine Whether TR4 is Upstream or Downstream of DNA Damage Signals.

Whether DNA damage checkpoint signals indicative of phosphorylation of p53, CHK2, H2AX, and ATM are impaired in TR4 KO cells is examined using specific phospho-antibodies against P53, CHK2, H2AX, and ATM upon UV and IR treatment. If these DNA damage checkpoint signals are lost in TR4 KO cells, it can concluded that TR4 can be upstream of p53, CHK2, H2AX, and/or ATM. Alternatively, genomic instability caused by loss of TR4 in TR4 KO cells leads to activation of p53, CHK2, H2AX, and/or ATM; in this scenario, TR4 is downstream of p53, CHK2, H2AX, and/or ATM.

(e) To Investigate Whether DNA Damage Signals Regulate TR4 Nuclear Translocation.

Predominant expression of TR4 in cytoplasm in LG and HG cancer as compared to its nuclear expression in normal prostate indicates that nuclear import/export of TR4 plays an important role in prostate cancer. A determination as to whether DNA damage signals induce TR4 nuclear translocation is made. Chamber-slide seeded wt MEFs and H1299 cells are treated with or without IR (6 Gy), or UV (100 J/m2), or H₂O₂ (200 mM) and fixed with 3% paraformaldehyde 0, 1, 4, 8, and 12 hs after the treatment. Cells are immunostained using a specific mouse monoclonal anti-TR4 antibody and a FITC-conjugated secondary antibody to determine nuclear/cytoplasmic localization. Western blotting using nuclear and cytoplasmic extracts is used to determine nuclear/cytoplasmic localization of TR4 upon the stresses. To determine whether the phosphorylation status of TR4 affects TR4 subcellular localization, the kinase/phosphotase activator/inhibitors are applied. To confirm this, GFP-TR4 and GFP-TR4 mutants at Ser-351 and Ser-144 sites are constructed and tested for their cellular localization under stresses.

(f) Detailed Methods:

(i) Nuclear Run-On Assay.

Wt MEFs and H1299 cells are treated with or without IR (6 Gy), or U (100 J/m2) or H₂O₂ (200 mM), and the nuclei are harvested 4, 8, and 12 hs after treatment. RNA transcripts are labeled by 32P and isolated using a Qiagen RNeasy kit and hybridization are carried out at 55° C. Mouse and human TR4 cDNA are prepared by PCR and full-length mouse or human β-actin cDNA are spotted onto Hybond N+ membrane (Amersham) using a dot-blot apparatus. The TR4 hybridization signals are quantified using a phosphorimager (Bio-Rad) and normalized with the β-actin signal.

(ii) Actinomycin D and -Amanitin.

Wt MEFs and H1299 cells are treated with or without IR (6 Gy), U (100 J/m2), or H₂O₂ (200 μM), and with 5 g/ml of Actinomycin D or 100 ng/ml of b-Amanitin. Cells are harvested at 4, 8, and 12 hs after treatment and total RNA are isolated. TR4 mRNA levels are quantified by Q-PCR and normalized with the β-actin signal.

(iii) A 6 kb TR4 Promoter.

The modulation of TR4 activity can be achieved via regulation of TR4 expression levels, therefore study of the 5′TR4 reveals how genotoxic stress influences TR4 activity. Based on the findings, TR4 mRNA was up-regulated upon IR, which indicated that the TR4 promoter contains a stress-responsive element (SRE) corresponding to the stress. A 6 kb TR45′-flanking region and its serial deletions have been cloned and constructed into Luciferase reporter genes. The transcriptional activity on the 5′TR4-Luc and its serial deletions upon stresses (UV, IR, and H₂O₂) are tested to determine the SREs.

(iv) Cycloheximide.

Wt MEFs and H1299 cells are treated with or without IR (6 Gy), or U (100 J/m2), or H₂O₂ (200) in the presence of cycloheximide (5 μg/ml). The cells are harvested 4, 8, and 12 hours after IR, UV irradiation or H₂O₂ (200 μM), and total protein are isolated and TR4 protein expression are analyzed by Western blotting using a monoclonal TR4 antibody. Quantification are done using ECL-chemiluminescence (BioRad) and normalized with the β-actin signal.

(v) Determination of Nucleic- and Cytoplasic-TR4 by Western Blotting:

Cells are suspended in 2 ml MS buffer (210 mM mannitol, 70 mM sucrose, 5 mM Tris-HCl/pH 7.5 and 1 mM EDTA) containing protease inhibitor cocktail (Boehringer Mannheim GmbH), homogenized using a homogenizer and then centrifuged at 1300 g at 4 C for 10 mins to get pellet nuclei and unbroken cells with cytoplasm in the supernatant. The pellet are washed with ice-cold PBS and resuspended in MS buffer. The nuclei are further purified by a second round of centrifugation at 1300 g. The protein concentration are determined and subjected into Western blotting analysis for TR4 expression.

(13) Summary:

The goal for this study is to identify the role of TR4, a longevity assurance gene, in tumorigenesis through maintaining the genomic stability. TR4 represents the first member of the nuclear receptor superfamily whose physiological functions directly link to aging and cancer. TR4, like other members of steroid receptor family, can be activated/modulated by their up-stream regulators.

### Example 3

### Modulation of Radiation Sensitivity by Testicular Orphan Receptor 4

Radiation therapy relies on the free radical disruption of cellular DNA. Living organisms are armed with different strategies to respond to radiation-induced DNA damages and the outcome of such results in radiation sensitivity. Prediction of radiation sensitivities of cancer cells is desired to determine the therapeutic course before radiation therapy. Therefore, understanding the mechanisms of DNA repair and the signaling pathways involved in radiation sensitivity are the key elements to modulate cellular radiation sensitivity. With advanced technology, genomic approaches have been extensively applied to identify novel molecular markers which can serve as an index of intrinsic cellular radiation sensitivity. However, the use of microarray technology in the process of gene discovery has been hindered by the complexities of gene network systems in response to IR. Different approaches such as the combination of gene profile analysis and genetic modified animal models can enhance the understating of IR response and better prediction of IR clinical outcomes, even lead to the development of a novel strategy to increase the IR sensitivity.

(1) Radiation-Sensitivity.

Cells have evolved several protective responses to counteract the harmful effects of IR-induced DNA damage. Extensive DNA damage can result in cell death (radiation-sensitivity), which can occur through several routes. Cells can choose to enter a state of irreversible growth arrest (replicative death) as measured by clonogenic survival, or apoptosis, which is a tightly regulated process that involves the interactions of a variety of proteins. An alternative protection mechanism involves the combination of cell-cycle checkpoints and DNA-damage repair. Following DNA damage, cells can activate cell cycle checkpoints that allow the cell to pause at the cell cycle G1/S or G2/M boundary, preventing replication of damaged DNA. DNA-repair pathways can restore the integrity of the DNA during this time. Fidelity of repair is important to the fate of the cell, as inaccurate repair can lead to mutations and genomic instability that can contribute to carcinogenesis.

It is thought that DNA repair is related to cell cycle progression, and cell cycle arrest in response to IR determines sensitivity to damage. The cell cycle plays an important role in the determination of radiation survival, as cycling cells exhibit various degrees of sensitivity that appear to be cell cycle-phase specific as well as tissue specific. It has been established that activation of the G1 and G2/M phase checkpoints in response to DNA damage alter radiation sensitivity (Little J B 1994 Radiat Res 140:299-311). Radiation sensitivity is a complex biological phenomenon that is influenced by a variety of factors including DNA repair, and changes in cellular metabolism and interactions. Tumor oxygenation, growth rate, cell-cycle distribution, and gross repair capacity also affect survival to radiation treatment.

(2) IR Induced Double-Strand DNA Break Repair.

Two types of repair exist in response to DSBs produced by IR-induced DNA damage. They are homologous recombinational repair (HRR) and non-homologous end-joining (NHEJ).

(a) NHEJ:

NHEJ requires the activities of Ku70/Ku80(86), DNA-PKcs, Lig4, and XRCC4, and is intrinsically more error prone than HRR, which utilizes an undamaged sister-chromatid as a template. DSBR usually follows bi-phasic kinetics with a fast (t50: 5-30 min) DNA-PK dependent component and a slow (t50: 1-10 h) relatively uncharacterized component, that is dependent on the Rad52 epistasis group of proteins (Wang et al., 2001). It is also thought that NHEJ predominates in the early phases of the cell cycle, namely G1 and S, while HRR functions in replicating cells (Takata M, et al. 1998 Embo J 17:5497-508).

The catalytic component of NHEJ, DNA-PK holoenzyme, consists of a 350 kDa catalytic subunit (DNA-PKcs) and a heterodimer of Ku proteins. DNA-PKcs is a protein Ser/Thr kinase whose activity is dependent on the availability of dsDNA ends. Ku is an abundant nuclear heterodimer of 70 and 80(86) kDa that binds to free DNA ends. The association of Ku 70/80 with broken DNA appears to recruit DNA-PKcs to the damaged ends of DNA resulting in the formation of active DNA-PK repair machinery at the site of DNA lesions (Salles-Passador I, et al. 1999 C R Acad Sci III 322:113-20). Mutations in DNA-PK observed in M059J, xrs5 and CHOV3 cell lines, and SCID (severe combined immuno-deficient) mice lead to increased radiation-sensitivity (Peterson S R et al. 1997 J Biol Chem 272:10227-31). In higher eukaryotes, mutations in Ku70, Ku80, and DNA ligase IV increase cell radiation-sensitivity to killing and compromise rejoining of IR-induced DSBs (Takata M, et al. 1998 Embo J 17:5497-508)

(b) HRR:

In addition to NHEJ, HRR has an important role in the repair of DSBs. In contrast to NHEJ, conservation HRR utilizes Holiday junction formation to facilitate strand transfer exchange between sister chromatids, and is therefore less error-prone (Thompson LH, Schild D 2001 Mutat Res 477:131-53). There are two types of HRR: single-strand annealing (SSA) and homology-directed conservative recombination. SSA is Rad51-independent, and involves the Rad50/Mre11/NBS1 complex, while conservative recombination requires the action of members of the Rad52 epistasis group, including Rad51/Rad52/Rad54, in addition to the involvement of XRCC2 and XRCC3 (Grenon M, et al. 2001 Nat Cell Biol 3:844-73; Liu Y, Kulesz-Martin M 2001 Carcinogenesis 22:851-60; Krejci L, et al. 2001 Mol Cell Biol 21:966-76). Recently, it has been implicated that a variety of other proteins, including tumor suppressor gene proteins BRCA1, and BRCA2, are involved in Rad51 binding and nuclear foci formation (Marmorstein L Y, et al. 1998 Proc Natl Acad Sci USA 95:13869-74; Haaf T, et al. 1999 J Cell Biol 144:11-20; Chen Y, et al. 1999 J Cell Physiol 181:385-92; Tashiro S, et al. 2000 J Cell Biol 150:283-91).

Rad51 is a ssDNA-dependent ATPase involved in DNA binding, co-localization to mitotic nuclei, and the formation of filaments on ssDNA that allow strand exchange (Thompson L H, Schild D 2001 Mutat Res 477:131-53). Rad51 also exhibits 5′ to 3′ exonuclease activity and requires the action of the ssDNA binding protein, Replication Protein A (RPA), to catalyze the strand transfer reaction (Sturzbecher H W, et al. 1996 Embo J 15:1992-2002). In cells lacking BRCA1, no Rad51 foci were observed in S phase cells (Scully R, et al. 1997 Cell 88:265-75). Rad51 foci also do not form in XRCC3 mutant cells lines, confirming the involvement of a variety of proteins in HRR (O'Regan P, et al. 2001 J. Biol Chem 276:22148-53).

Several lines of evidence indicate a role for BRCA1 in DNA damage-induced repair. BRCA1 has been found to interact with various proteins involved in DSBR, including Rad50, Rad51 and BRCA2. BRCA1 is a known transcription factor that has been found to interact with p53 and stimulate p53-dependent transcription of the p21 promoter (Deng C X, Brodie S G 2000 Bioessays 22:728-37). In addition, BRCA1-deficient cells are also hypersensitive to IR, which can be partially rescued by a mutation in p53, implicating the involvement of p53 in this process (Moynahan M E, et al. 2001 Cancer Res 61:4842-50; Xu X, et al. 2001 Nat Genet. 28:266-71). Other factors involved in HRR include the Rad51 paralogs Rad51b/c/d and XRCC2/3, which share approximately 30% homology with the Rad51 protein (Thompson L H, Schild D 2001 Mutat Res 477:131-53). These proteins have similar but non-overlapping functions with Rad51. Rad51b, which is induced by both gamma- and UV-irradiation, has not been shown to have recombinase activity, although over-expression leads to a G1 delay. Interestingly, Rad51b has kinase activity and can phosphorylate kemptide, myelin basic protein, p53, cyclinE and cdk2, supporting its involvement in the cell cycle response to damage (Havre P A, et al. 2000 Exp Cell Res 254:33-44). Rad51d is a DNA-stimulated ATPase that binds ssDNA and forms a complex with XRCC2 directly (Braybrooke J P, et al. 2000 J Biol Chem 275:29100-6) Less is known about Rad51c, which has been found to interact with XRCC2 in 1:1 stoichiometry from two hybrid screening using a cDNA library (Kurumizaka H, et al. 2001 Proc Natl Acad Sci U S A 98:5538-43).

Another member of the HRR group, Rad52, shows annealing activities and promotes the exchange of RPA for Rad51 protein on ssDNA (Sung P 1997 J Biol Chem 272:28194-7). Human Rad52 has been shown to bind DSBs (Krejci L, et al. 2001 Mol Cell Biol 21:966-76). In addition, Rad54 belongs to a SWI2/SNF2 protein family, whose members are involved in the modulation of chromatin structure (Krejci L, et al. 2001 Mol Cell Biol 21:966-76). Biochemical studies show that Rad54 binds DNA and promotes Rad51-dependent homologous DNA pairing through changes in DNA double-helix conformation (Sung P 1997 J Biol Chem 272:28194-7). Together, the interactions between Rad54 and Rad52 and the binding of p53 to Rad51 allow the HRR machinery to localize at the site of DNA damage (Thompson L H, Schild D 2001 Mutat Res 477:131-53).

(3) Role of p53 in DSBR.

p53 is thought to function in the maintenance of genomic stability by sequence non-specific DNA binding to sites of damage and subsequent interaction with a variety of cellular proteins involved in the repair of DNA damage (Liu Y, Kulesz-Martin M 2001 Carcinogenesis 22:851-60). Due to the large variety of environmental, chemical, and physiological DNA damaging agents, it is important to understand the mechanism by which the cell responds. It has been shown that p53 suppresses HRR and activates NHEJ, both in vitro and in vivo, suggesting multiple roles for p53 in DNA repair regulation (Bill C A, et al. 1997 Mutat Res 385:21-9; Brown K D, et al. 2000 J Biol Chem 275:6651-6; Mekeel K L, et al. 1997 Oncogene 14:1847-57; Tang W, et al 1999 Cancer Res 59:2562-5; Wiesmuller L, et al. 1996 J Virol 70:737-44).

It has recently been shown that ATM kinase activity is necessary for Rad51 and Rad54 foci formation in response to IR(Morrison C, et al. 2000 Embo J 19:463-71). Foci were not formed at the sites of DNA damage in ATM null cells, implicating the importance of ATM in damage recognition. ATM is necessary for recognition of DSBs, and initiates a series of key phosphorylation events that result in the activation of p53, and subsequent binding of p53 through its carboxy-terminus to broken DNA. In this manner, p53 can localize to the sites of DNA damage and interact with the machinery involved in DSBR.

Recent data also indicate that wild-type p53 is necessary for DNA-PK activity in NHEJ. It has been shown that p53 is required for the post-irradiation rise in cellular Ku70 protein levels, as well as nuclear localization of the protein in asynchronous cells (Brown K D, et al. 2000 J Biol Chem 275:6651-6). In addition, p53 has been found to directly enhance rejoining in asynchronous IR treated fibroblasts (Tang W, et al 1999 Cancer Res 59:2562-5). The role of p53 in NHEJ has not been studied in synchronized cell cycle-phase populations.

Recent studies suggests that proteins involved in DSBR may be inactivated during the onset of apoptosis, further providing a role for p53 in the regulation of DSBR. It has been shown that the Rad51 recombinase is cleaved in mammalian cells by caspase 3, one of the main executors of apoptosis, following IR exposure (Huang Y, et al. 1999 Mol Cell Biol 19:2986-97). In addition, it has been shown that protein kinase C8 is activated at the onset of apoptosis and inhibits DNA-PKcs by phosphorylation (Bharti A, et al. 1998 Mol Cell Biol 18:6719-28). This event prevents association of DNA-PKcs with DNA and inhibits its kinase activity, providing a mechanism for the regulation of DNA repair by p53. These data support the model by which p53 regulates the balance between repair and death in H1299/p53 cells.

(4) UV-Induced Nucleotide Excision Repair:

Nucleotide excision repair (NER) is a major pathway for repairing bulky DNA lesions upon exposure to UV and chemicals. Repair of damaged DNA includes (a) recognition, (b) excision and removal of DNA lesions, (c) new nucleotide synthesis, and (d) ligation of newly synthesized DNA back to the genome. Key proteins in the human NER pathways include: (a) XPA, RPA, and XPC that are responsible for DNA binding and damage recognition, with the aid of XPB, XPD, and several proteins in the TFIIH complex that unwind the DNA duplex and are involved in kinetic proofreading, (b) XPG and XPF-ERCC1 complex that are nucleases in incising DNA damaged lesions at 5′ and 3′ phosphodiester bonds, (c) DNA Polδ/ε, PCNA, and RCF that fill in the gap with new DNA synthesis, and (d) DNA ligase I. In addition, CSA and CSB mediate transcription-coupled DNA repair in which transcribing strand is repaired at a faster rate.

Given the importance of NER in defending genome integrity, it is not surprising that mutants in the NER pathway lead to serious diseases. Indeed, hereditary disease xeroderma pimentosum (XP) is characterized by a 10,000-fold increased risk of skin cancer. Mutations in both human and murine XPD gene also lead to trichothiodystrophy, characterized with brittle hair and nails, and a shorter lifespan.

b) Results:

(1) TR4 KO Cells Show More Sensitivity to IR than TR4 Wt Cells.

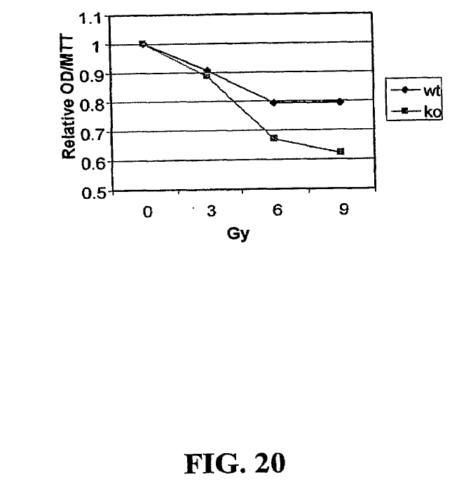

To reduce the ability to tolerate IR is a hallmark of radiotherapy. To test whether loss of TR4 change the sensitivity of cells to IR how wt, TR4 heterozygous and TR4 KO MEFs respond to IR was examined. As shown in FIG. 20, TR4 KO MEFs are more sensitive to IR, and fewer TR4 KO MEFs survive compared with wt MEFs.

(2) TR4 Expression is Induced While Cells Expose to IR.

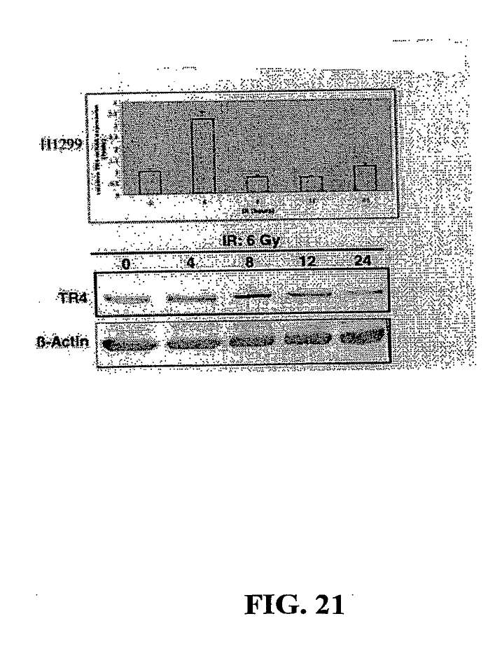

If TR4 mediates repair of DSBs, it is possible that TR4 expression is upregulated in response to IR. Indeed, it was found that TR4 mRNA and protein levels increased between 4 and 8 hours after IR in H1299 cells (FIG. 21). This result indicates that TR4 expression is tightly regulated in response to IR. The molecular mechanisms by which TR4 mRNA and protein are upregulated by IR is disclosed herein.

(3) Expression of a Hypo-Phosphorylated TR4 was Increased in a p53 Independent Manner Upon UV Irradiation.

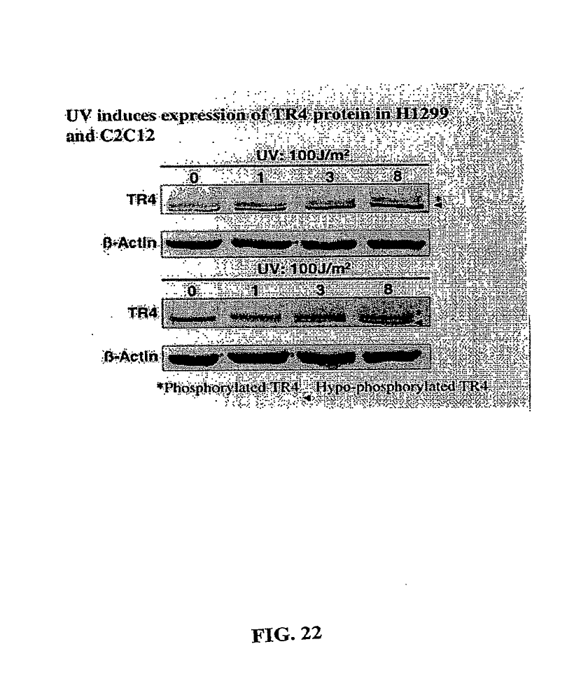

The results showing that TR4 induces repair of UV-damaged DNA led to testing whether TR4 expression is induced upon UV irradiation. Using a specific monoclonal TR4 antibody, it was found that TR4 protein expression is increased significantly one hour after UV irradiation in both human H1299 and mouse C2C12 cells (FIG. 22). Notably, two bands were recognized by the TR4 antibody and it is the faster migration band that shows increased expression in response to UV (FIG. 22). This indicates that the detection of two bands by the TR4 antibody is due to differently phosphorylated forms of TR4. Indeed, when cell extracts were treated with alkaline phosphatase, a single and faster migration band was detected by the TR4 antibody. These results indicate that TR4 is a phosphorylated protein and a hypophosphorylated form of TR4 is induced upon UV irradiation. Furthermore, H1299 is a p53 null cell line, indicating that the hypophosphorylated TR4 was induced by UV in a p53 independent manner,

(4) Dephosphorylation of TR4 at S351 is Required for UV-Induced DNA Repair.

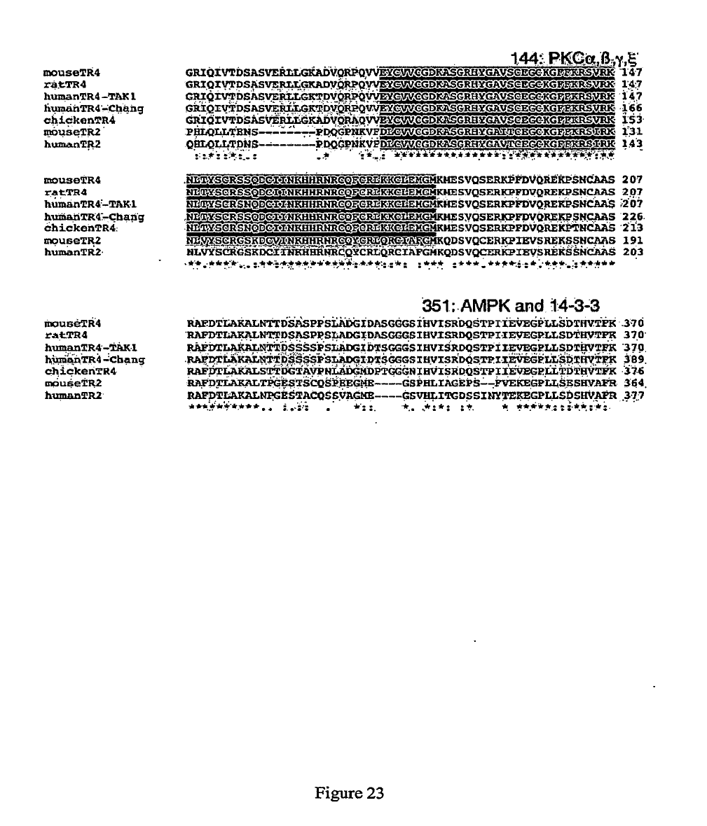

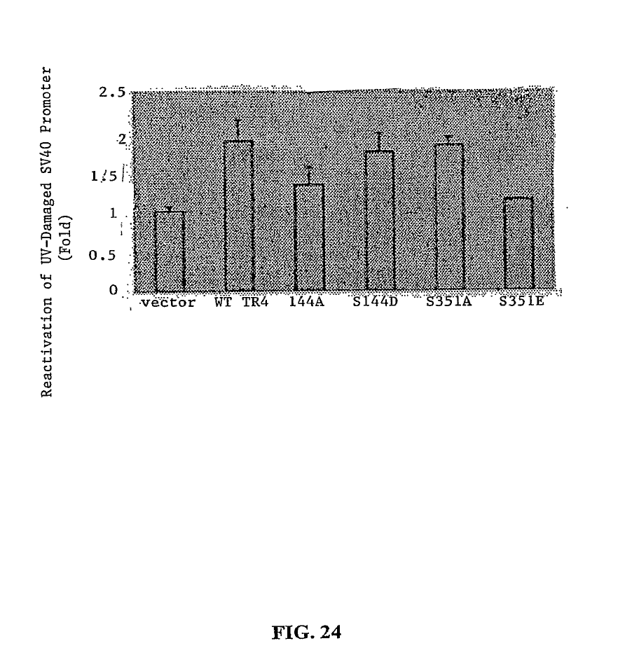

To study how TR4 activity is regulated by phosphorylation, putative phosphorylation sites were found using a computer program at MIT (http://scansite.mit.edu). It was observed that Serine at 351 (Ser-351) is a highly stringent binding site for 14-3-3 and is conserved from mouse to human (FIG. 23). To test whether phosphorylation of Ser-351 in TR4 plays a role in UV-damaged DNA repair, Ser-351 was mutated to Alaline (S351A) that mimics a dephosphorylated form of TR4 at Ser-351 and to Glutamic acid (S351E) that mimics a phosphorylated form of TR4 at Ser-351, and measured their efficiency in repairing UV-damaged DNA. S351A mutant induced DNA repair effectively and conversely, the DNA repair is abolished with the S351E mutant (FIG. 24). These results indicate that dephosphorylation of TR4 at Ser-351 is essential for inducing UV-damaged DNA repair.

(5) Phosphorylation of TR4 at S144 Induces Repair of UV-Damaged DNA

The scansite program also identified Serine-144 (Ser-144) of TR4 as a highly stringent phosphorylation site for PKCα, β, γ, and ξ, (FIG. 24), which has been implicated in playing a role in NER and NER pathways. However, molecular mechanisms by which for PKCα, β, γ, and ξ regulate BER and NER are not clear. To test whether phosphorylation of Ser-144 is required in UV-damaged DNA repair, a S144A in which Ser-144 is mutated to Alanine was made and a S144D mutant that mimics constitutively phosphorylated form of TR4 at Ser-144 generated by changing Ser to aspartic acid (1), and analyzed the capacity of S144A and S144D in UV-damaged DNA repair. Expression of S144A reduces DNA repair efficiency as compared to wt TR4; in contrast, S144D effectively induced DNA repair (FIG. 24). These results indicate that phosphorylation Ser-144 is required for repairing UV-induced DNA damage. Using both in vivo and in vitro immunoprecipitation and GST pull down assays, whether Ser-144 is phosphorylated by PKCα, β, γ, and ξ, in response to UV is tested.

(6) TR4 Induces CSB Expression in Transcription Coupled Nucleotide Excision Repair.

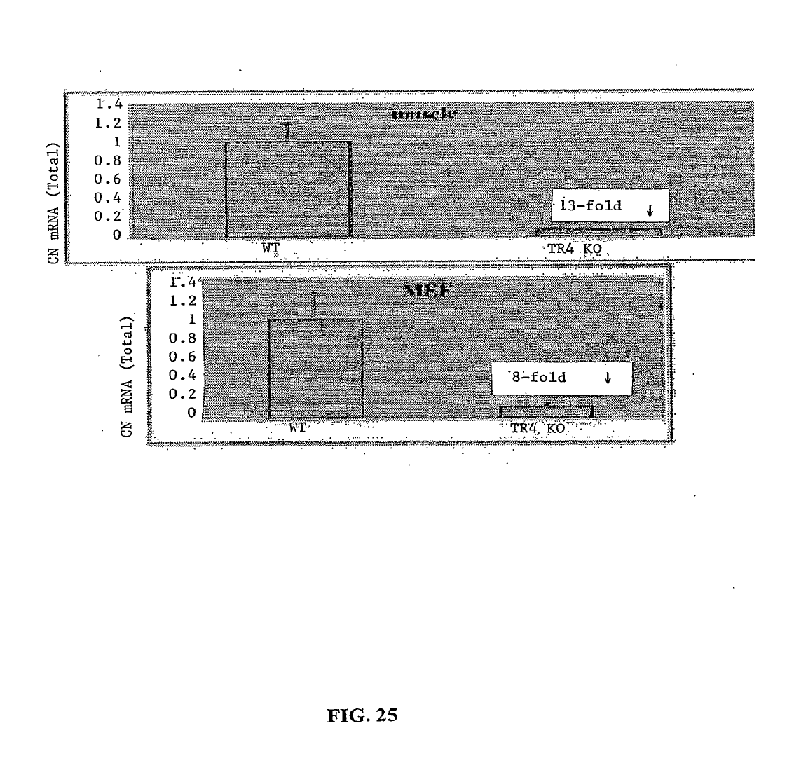

The above results indicate that TR4 regulates UV-damaged DNA repair, presumably by activating the nucleotide excision repair (NER) pathway. To understand the molecular mechanisms by which TR4 regulates the NER pathway, the expression of a battery of NER genes was examined in TR4 KO tissues and MEF cells. Cockayne syndrome protein B (CSB) but not other NER genes, including CSA, XPA, XPD, XPC XPF, XPG, ERCC1, and DDB2, was dramatically reduced in TR4 KO MEFs and 5-week-old TR4 KO muscle as compared to the wt mice controls (FIG. 25). This result indicates that CSB expression is dependent on TR4. Activation of CSB expression by TR4 could also explain that the role of TR4 in UV-damaged DNA repair is primarily involved in transcription coupled repair (TCR), but not global genomic repair (GGR) (FIG. 25). The role of TR4 in TCR or GGR is confirmed by using GGR immunoassay (CPDs) and strand-specific repair assay. Furthermore, whether TR4 directly regulates CSB expression is tested by studying the CSB promoter and an inducible TR4 and TR4 RNAi system in CV-1 and H1299 cells.

(7) TR4 Regulates Nonhomologous End Jointing.

To test whether TR4 is involved in nonhomologous end jointing (NHEJ) in response to DSBs, a newly developed NHEJ assay system was used (Seluanov A, et al. 2004 Proc Natl Acad Sci USA 101:7624-9). As shown in FIG. 16 it was found that cells expressing TR4 had a slightly higher NHEJ activity than the control cells expressing empty vector only, indicating that TR4 mediates DNA repair in response DSBs. Also, to improve the sensitivity of this NHEJ assay in primary cells such as MEFs, an improved cell transfection system, Amaxa Nucleofactor, is used to increase cell transfection efficiency. Ultimately, whether loss of TR4 reduced NHEJ activity is tested by using TR4 KO MEFs and/or wt MEF cells expressed with TR4 RNAi. Furthermore, whether replacing TR4 expression through the use of retrovirus system in TR4 KO cells restores NHEJ repair is tested.

(8) The Structure and Functional Study of TR45′ Promoter:

To investigate how stress influences TR4 expression at transcriptional level, a 6.0 kb genomic DNA fragment containing the TR4 gene promoter region was cloned, sequenced, and characterized. Sequence homology search within this promoter region revealed potential cis-acting elements that can be recognized by several transcriptional factors such as GR, C/EBP, SP1, YY1, and MyoD. Deletion analyses and Luciferase assay showed a potential enhancer element, within 216 to 167 bp upstream of the transcription start site (FIG. 11), which is associated with the TR4 transcriptional activity.

(9) Construction of TR4 RNAi:

As shown in FIG. 16, three TR4 RNAi were constructed into pSuperior.retro.puro (OligoEngine) vector, and their ability to suppress TR4-mediated TR4RE-Luc activity was tested. Clone 2-9 TR4 RNAi showed a better suppression effect, and are used in the studies.

c) Experimental Design:

(1) Identification of the Signaling Pathways by which Radiation Modulates TR4 Expression and Activity.

Loss of genome stability due to malfunctioned DNA repair machineries can lead to premature aging. The expression of TR4, a transcriptional factor, is induced by radiation, and the activation of TR4 reduces DNA damage; therefore the inactivation of TR4 results in genomic instability and leads to premature aging. It is disclosed herein that TR4 is a stress responsive molecule that is up-regulated in response to DNA damage checkpoint signals, that it promotes cellular defense signals to protect cells from DNA damage. In order to investigate TR4 roles on the cellular surveillance defense systems, the alterations of TR4 expression in response to radiation is measured, and these changes to TR4 transactivation activity and to TR4-mediated biochemical signal transduction pathways within the cell defense systems are correlated. Moreover, the molecular mechanisms underlying how radiation stimulates TR4 activity are determined. The regulation of TR4 activity can be achieved via regulation of TR4 expression levels as well as via post-translational modification of TR4. Therefore, the 5′-promoter of TR4 is studied to reveal how IR-induced regulatory factors change TR4 expression, as well as to determine how TR4 is modified by IR-induced kinase cascade in response to IR.

(2) TR4 mRNA is Up-Regulated at the Transcriptional and/or Post-Transcriptional Levels (mRNA Stability).

Nuclear run-on assays are performed to test whether UV and/or IR affects TR4 at transcriptional level. TR4 transcription rate are measured using nuclei from both irradiated cells and non-irradiated control cells. In addition, to measure TR4 mRNA stability, the cells are treated with transcriptional inhibitors, actinomycin or α-Amanitin, and TR4 mRNA expression quantitated by real-time PCR. Furthermore, TR4 gene regulation is studied by dissecting the 6 kb of TR45′-promoter.

(a) Detailed Methods:

(i) Nuclear Run-on Assay.

Wt MEFs and H1299 cells are treated with or without IR (6 Gy), or U (100 J/m²), and the nuclei are harvested 4, 8, and 12 hours after IR or UV irradiation. RNA transcripts are labeled by P³² and isolated using a Qiagen RNeasy kit and hybridization are carried out at 55° C. Mouse and human TR4 cDNA are prepared by PCR and full-length mouse or human β-actin cDNA are spotted onto Hybond N+ membrane (Amersham) using a dot-blot apparatus. The TR4 hybridization signals are quantitated using a phosphorimager (Bio-Rad) and normalized with the β-actin signal.

(ii) Actinomycin D and α-Amanitin.

Wt MEFs and H1299 cells are treated with or without IR (6 Gy), or U (100 J/m²), and with 5 μg/ml of Actinomycin D or 100 ng/ml of α-Amanitin. Cells are harvested 4, 8, and 12 hours after IR or UV irradiation and total RNA are isolated. TR4 mRNA levels are quantitated by real-time PCR using a Q-PCR machine (Bio-Rad) and normalized with the β-actin signal.

(iii) A 6 kb TR4 Promoter.

The modulation of TR4 activity can be achieved via regulation of TR4 expression levels, therefore study of the 5′ promoter of TR4 reveals how genotoxic stress influences TR4 activity. Based on the preliminary findings, TR4 mRNA was up-regulated upon IR, which indicated that the TR4 promoter contains a stress-responsive element (SRE) corresponding to the stress. A 6 kb TR45′-promoter region and its serial deletions have been cloned and constructed into Luciferase reporter genes. The transcriptional activity on the 5′-TR4-Luc and its serial deletions upon irradiation (UV, and IR) is tested to determine the SREs.

(3) TR4 Protein is Up-Regulated at the Level of Translation and/or Post-Translation (Protein Stability).

TR4 protein expression is measured after cells are treated with or without irradiation, and with a translation inhibitor, cycloheximide to determine whether up-regulation of TR4 protein upon UV radiation and IR is at the translational level.

(a) Detailed Methods:

(i) Cycloheximide.

Wt MEFs and H1299 cells are treated with or without IR (6 Gy), or UV (100 J/m²) and then with cycloheximide (5 μg/ml). The cells are harvested 4, 8, and 12 hours after IR or UV irradiation and total protein are isolated and TR4 protein expression are analyzed by Western blotting using a monoclonal TR4 antibody. Quantitation are done using ECL-chemiluminescence (Biorad) and normalized with the β-actin signal.

(4) TR4 Activity in DNA Repair is Modulated Through Protein Phosphorylation Observed Using TCR and NHEJ Assays and Global Genomic NER Immunoassay.

Herein is disclosed that TR4 is a phosphoprotein and is dephosphorylated at least upon UV irradiation. Furthermore, the TR4 mutation analysis indicates that phosphorylation of Ser-144 and dephosphorylation of Ser-351 increase TR4 DNA repair activity in TCR. Ser-351 is a highly stringent binding site for 14-3-3, and Ser-144 is a highly stringent phosphorylation site for PKCα, β, γ, ξ. It is disclosed herein that TR4 can be modulated by kinase/phosphotase cascades involved in cell DNA-damage repair systems. This is shown by activating or inactivating putative kinases/phosphatases using pBabe, RNAi systems, and constitutively active and dominant negative forms of kinases, and testing the activity of TR4 in TCR and NHEJ. Furthermore, in vivo and in vitro interaction and kinase assays between TR4 and putative kinases/phosphatases are performed. First, CoIP of TR4 and kinases/phosphatases/interaction-proteins are performed to test their interaction. When the interaction between TR4 and kinases is found, in vitro kinase assays are performed.

(a) Detailed Methods:

(i) Determination of TR4 Expression by Real-Time PCR (Q-PCR) and Western Blotting Analyses.

MEFs from wt mice are treated with different doses of H₂O₂, V, and IR, and harvested at different time according to the designs described herein. The RNA samples are obtained by Trizol reagents, and total RNA are converted into first strand cDNA by SuperScript III reverse transcriptase (Invitrogen). Primers for amplification of TR4 are designed by the Becon Primer Designs software. Q-PCR is performed using Bio-Rad iQ cycler. CT values are calculated and normalized to the level of the housekeeping gene α-microglobulin. Relative gene expression are calculated according to 2^{-ΔΔCT} from three independent experiments. To confirm the expression changes in protein level, cells are lysed by RIPA buffer and quantified. Proteins are separated by 12% SDS-PAGE and blotted with anti-TR4 antibody (#15 monoclonal antibody) to detect changes in TR4 expression levels upon stress. In addition to MEFs, TR4 expression level is examined in response to a DNA-damage inducer in H1299 cells (expresses high levels of TR4) and CV-1, and C2C12 (express low amounts of TR4).

(ii) Cell Proliferation Assays:

Cell proliferation rate are determined by ³H-tymidine incorporation analyses and MTT assays. The response to stress between the TR4 KO MEFs and wt MEFs are compared as shown by percentage of cell survival upon low to high doses of stress treatment. Stress-treated surviving cells are calculated as the ratio of cell number in treated group to non-treated group. For ³H-tymidine incorporation analysis, cells are incubated for 24 h with medium containing 0.25 μCi/ml ³H-thymidine. The radioactivity incorporated is measured by liquid scintillation counting. For MTT assay, the conversion of a colorless substrate to reduced tetrazolium by the mitochondrial dehydrogenase, are used to assess cell viability and growth. After each treatment period, 10% volume of medium of thiazolyl blue (5 mg/ml, Sigma) are added into each well for 2-3 h at 37° C. The resultant precipitate is dissolved in 0.04 M HCl in isopropanol and absorbency are read at a wavelength of 570 nm with background wavelength at 660 nm.

(iii) DNA Damage Assays:

DNA single-strand breaks: A DNA precipitation assay is used for DNA-strand-breaks detection. Confluent MEF cells are labeled with 0.25 μCi/ml [3H] methylthymidine for 24 h. Cells are treated with various DNA-damage inducers. After treatment, the cells are washed with PBS and lysed with lysis buffer (10 mM Tris/HCl/10 mM EDTA/50 mM NaOH/2% SDS) followed by addition of 0.12 M KCl. The lysate are incubated for 10 min at 65° C. followed by a 5 min cooling-and-precipitation period on ice. A DNA-protein K-SDS precipitate is formed under these conditions, from which low-molecular-mass broken DNA is released. This DNA are recovered in the supernatant from a 10 min centrifugation at 200 g, 10° C., and transferred into a liquid scintillation vial containing 1 ml of 50 mM HCl. The precipitated pellet (intact double-stranded DNA) are solubilized in 1 ml of water at 65° C., the tube rinsed with 1 ml of water, and 8 ml of scintillation fluid added to each vial. The amount of double-stranded DNA remaining is calculated for each sample by dividing the d.p.m. value of the pellet by the total d.p.m. value of the pellet+supernatant and multiplying by 100. The results representing the extent of DNA damage are calculated as (Dt/Dc)×100, where Dt represents double-stranded DNA in treated cells and Dc represents double-stranded DNA in the respective control cells. In control cells (cells incubated in Ca2+-containing or Ca2+-free/EGTA), the level of total double-stranded DNA is around 75%. Pretreatment with various chelators did not affect this level (Jornot L, et al. 1998 Biochem J 335 (Pt 1):85-94).

(iv) Comet Assay:

An Fpg-FLARE (fragment length analysis using repair enzymes) comet assay kit is used in accordance with the manufacturer's instructions (Trevign, Ginthersberg, Mo.). This kit specifically detects oxidative DNA lesions such as 8-oxo-2-deoxyguanosine and formamidopyrimidines. Images of 50 randomly chosen nuclei per sample are captured using a CCD camera coupled to an epifluorescence microscope. Comet tail lengths are measured using the comet macro from NH public domain image analysis program.

(v) Transfection Assay and Luciferase Assays.

The 6 kb and serial deleted constructs with Luc reporter are transfected into CV1 cells, and then cells are treated with H₂O₂ (250 μM), UV, and IR. The region(s) that lose the response to H₂O₂-induced 5′-TR4-Luc activity are potential SREs and need to be analyzed further. More stress challenges, such as UV-irradiation, ionizing radiation, and low glucose are applied to determine the SREs within the TR45′-promoter. The putative SRE regions that are critical for stress response are further narrowed down by site-directed mutagenesis. The goal is to identify the minimal regions, around 30-50 bp, responsible for the stress-induced TR4. Transient transfection is performed by using SuperFect according to the manufacturer's suggested procedure (Qiagen). After transfection, cells are treated with 250 μM H₂O₂ for 2 hour, and then medium are replaced with fresh culture medium for 48 hour. Cell lysates are prepared and the luciferase activity is normalized for transfection efficiency using pRL-CMV as an internal control. Luciferase assays are performed using the dual-luciferase reporter system (Promega).

(vi) Site-Directed Mutagenesis of Potential SRE in TR4 Promoter.

If putative SREs, identified from the serial deletion TR45′-promoter study, contain some known cis-acting elements, the sequences in these cis-acting elements are mutated by using QuickChange Site-Directed mutagenesis kit (Strategene). If the regions identified contain no known cis-acting elements, the regions are mutated every 15-20 bp to narrow down the minimal regions for TR4 activation.

(vii) Construction of Gadd45 RNAi.

To generate Gadd45 RANi, the system is applied through OligoEngine (www.oligoengine.com) for specialized design software. The pSUPER vector is used to express the small RNA molecules to achieve long-term silencing of endogenous Gadd45a. Synthetic DNA oligos encoding two 19-nt reverse complements homologous to a portion of Gadd45a, separated by a short spacer region, are inserted into the vector. When expressed under the control of the polymerase-III based expression system, the RNA transcript forms a short hairpin structure with a 19 bp double-stranded region and two final uridines overhanging the 3′ end to generate siRNA for Gadd45 knockdown. After sequence confirmation, Gadd45 RANi are transfected and endogenous Gadd45 expressions (mRNA, and protein) are examined to determine RNAi efficiency. Two to three RNAi are designed and tested.

(viii) Site-Directed Mutagenesis to Generate TR4 Phosphorylation Site Mutants.

The putative phosphorylation site on TR4 is mutated by using QuickChange XL Site-Directed Mutagenesis kit (Stratagene). pCMX-TR4 are used as a template to be amplified by two primers containing the desired mutation by PfuTurbo DNA polymerase. Following the PCR cycle, the product is treated with Dpn I, which is used to digest the parental DNA template. The nicked vector incorporating the desired mutations is transformed into XL10-Gold competent cells, and clones are amplified and sequenced.

(ix) ChIP Assay:

CHIP is carried out using the Upstate Biotechnology (Charlottesville, Va.) ChIP assay kit with modifications. In brief, TR4-transfected cells are lyzed, cross-linked with 1% formaldehyde, and chromatin pellets are sonicated to an average of 200- to 1000-bp fragments of DNA. The chromatin fragments are subjected to immunoprecipitation with 2 μg TR4 antibody overnight at 4° C. The precipitates are eluted into the elution buffer containing 1% SDS, 100 mM NaHCO₃, and 10 mM DTT. The cross-links are reversed with a 4 h incubation at 65° C. in the elution buffer with addition of 200 mM NaCl. The immunoprecipitated DNA fragments are purified using QIAGEN MiniElute Reaction Cleanup kits and subjected to PCR using a pair of primers which were designed to amplify the Gadd45a promoter sequence containing DR3-VDRE.

(x) NHEJ Assay:

5 μg of GFP-Pem-Ad2 (NHEJ substrate) plasmid are digested by HindIII and are transfected into cells together with 1 μg of pDsRed (Clontech) by superfect (Qiagen) kit or Amaxa Nucleofector kit. Cells are incubated for 72 hours for GFP protein maturation, harvested and analyzed by flow cytometry using a FACS machine (Meckman Coulter, EPICS ELITE ESP). The ratio of GFP positive and DsRed positive cells are analyzed.

(xi) Global Genomic NER Immunoassay:

Repair of cytobutane pyridine dimers (CPDs) and 6-4 photoproducts are measured by an immunoblot assay using monoclonal antibodies specific for either CPDs or 6-4 photoproducts. Statistical analysis of differences in DNA repair is performed using the unpaired t-test.

(xii) Strand-Specific DNA Repair Assay:

Removal of CPDs between the transcribed and non-transcribed is determined by studying the dihydrofolate reductase (DHFR) gene. Strand specific RNA probes are used to measure the frequency of CPDs in a 17 kb and a 7 kb fragment in the central region of the DHFR gene, which are digested by HpaI and PstI restriction enzyme with or without bactriophage T4 endonuclease, respectively. Southern blotting analysis is used to measure DNA repair efficiency: better repair is indicated by the presence of more 17 kb and 7 kb fragments.

EMSA, and DNA pull-down assays follow the protocols described previously (Lee Y F, et al. 1998 J Biol Chem 273:13437-43; Bao B Y, et al. 2004 Oncogene 23:3350-60).

(5) To Determine the Molecular Mechanisms by which TR4 Regulates UV-Damaged DNA Repair.

Nucleotide excision repair is a major pathway for repairing bulky DNA lesions upon exposure to UV and chemicals. There are two pathways in NER: Little J B 1994 Radiat Res 140:299-311 transcription-coupled repair (TCR) repairs the engaging DNA strand during transcription and Takata M, et al. 1998 Embo J 17:5497-508 global genomic repair (GGR) regulates DNA repair independently of the transcription status of damaged DNA. In the previous studies using a DNA-damage reporter gene assay it was observed that TR4 induced the UV-damaged DNA repair, especially in the TCR pathway. By screening the genes involved in the NER pathway, it was found that some of the genes are reduced in TR4 KO mice, therefore, TR4 induces gene expression in nucleotide excision (NER) in response to UV irradiation.

(6) To investigate Whether Gadd45 and CSB Expression s Induced Through TR4 in Response to UV in a p53-Independent Manner.

The data indicate that TR4 is necessary for Gadd45 and CSB expression. To determine whether Gadd45 and CSB mRNA expression is dependent on TR4 upon irradiation, an inducible system is constructed to express TR4 cDNA or TR4 RNAi in order to induce TR4 expression and knock down TR4 expression, respectively. Furthermore, p53 is an important protein that mediates various DNA damage responses and the data indicate that TR4 expression can be induced in a P53 independent manner. Therefore, to further determine whether induction of Gadd45 and CSB by TR4 is p53-dependent, stable clones of pBIG-2I-TR4 (TR4 cDNA expression), pBIG-2I-TR4-RNAi (TR4 knockdown), or pBIG-2I (control empty vector) are selected by Hygromycin B (100 μg/ml) in CV-1 cells (low levels of endogenous TR4 and WT p53) and H1299 cells (high levels of endogenous TR4 and mutant dysfunctional p53). Stable clones are irradiated by UV (100 J/m²) with or without doxycycline (2 g/ml) to induce expression of TR4 cDNA or RNAi. The cells are harvested 4, 8, 12, 24 hs after UV irradiation and TR4 mRNA and protein expression are analyzed by real-time PCR and Western blotting using a TR4 monoclonal antibody.

(7) To Study Whether TR4 Directly Regulates CSB by Studying the CSB Promoter.

Both mouse and human CSB promoters are isolated, analyzed and tested whether they respond to TR4 overexpression or knockdown using reporter gene assay system in which CSB-promoter-luciferase (CSB-luc) are constructed. CV-1 cells are transfected with a CSB-luc plasmid together with PCMX or PCMX-TR4 and the luciferase activity are analyzed using a luciferase reader. Furthermore, EMSA and ChIP assays are performed to test whether CSB is directly regulated by TR4.

(8) To Confirm the Role of TR4 in DNA Repair Using TR4 RNAi.

The data indicate that TR4 is involved in TCR and NHEJ. Whether knockdown of TR4 using TR4 RNAi results in reduction of DNA repair is tested. TCR assay are performed on stable clones of CV-1 or H1299 cells carrying pBIG-2I-TR4-RNAi (TR4 knockdown), or pBIG-2I (control empty vector) transfected with UV-damaged SV40-luciferase and treated with or without doxycycline (2 μg/ml). Reactivation of luciferase activity is analyzed as an indicator of DNA repair efficiency.

(9) To Confirm the Role of TR4 in TCR or GGR by Using GGR Immunoassay (CPDs) and Strand-Specific Repair Assay.

Also, the role of TR4 in TCR is confirmed using GGR immunoassay (CPDs) and strand-specific repair assay. Repair of the transcribed and non-transcribed strands in TCR as well as GGR are analyzed. Both assays are performed using TR4 KO as well as wt MEFs.

(10) To Determine Whether TR4 Genetically Interacts with Gadd45 and CSB in DNA Repair.

(a) To Determine Whether DNA Repair Phenotypes of TR4 KO Cells are Rescued by Expressing Gadd45 and/or CSB.

As shown herein, TR4 regulates Gadd45a-5′-promoter containing Luc reporter gene activities in a TR4-dose dependent manner in transient transfection assays. It is disclosed herein that TR4 can protect cell from DNA-damage through, at least, partial mediated up-regulation of Gadd45a. Whether the decreased DNA damage protective effects in TR4 KO can be restored is tested by restoring Gadd45a in TR4 KO cells using TCR and GGR assays. Meanwhile, whether CSB restoration in TR4 KO cells results in better DNA repair activity is tested.

(b) To Determine Whether Loss of Gadd45 and/or CSB in TR4 KO Cells Compromise DNA Repair Mediated by TR4.

TR4 can protect cell from DNA-damage through, at least, partial mediated up-regulation of Gadd45a and CSB. To further confirm this, blocking endogenous Gadd45a and/or CSB by RNAi are used to test if the cells lose the TR4 protecting effects. MEFs from wt cells are stably transfected with Gadd45a and/or CSB RNAi (p-super vector) and scramble RNAi control and then test their response to genotoxic challenge. If TR4, Gadd45, and CSB mediate DNA repair in the same linear pathway, it is likely that DNA repair activities are not further compromised when these proteins are all knocked out and/or down in the same cells. Alternatively, Gadd45 and CSB may be two out of many downstream targets of TR4 in DNA repair; in this case, triple knockdown or knockout of these three genes result in more severe deficiency in DNA repair than the double knockdown or knockout of any two of these three genes. TCR and GGR assays are used. Alternatively, Gadd45 KO and CSB cells expressed with TR4 RNAi can be used.

(c) Detailed Methods:

(i) Establishment of Inducible TR4 and TR4 RNAi Stable Cell Lines.

Stable clones of pBIG-2I-TR4 (TR4 cDNA expression), pBIG-2I-TR4-RNAi (TR4 knockdown), or pBIG-2I (control empty vector) are selected by Hygromycin B (300 g/ml) in CV-1 cells (low levels of endogenous TR4 and wt P53), COS-1 (modest levels of TR4) and H1299 cells (high levels of endogenous TR4 and mutant dysfunctional P53). Stable clones are treated with doxycycline (2 μg/ml) to induce expression of TR4 cDNA or RNAi.

(ii) UV-Irradiation Cells:

Cells which are described in the previously are seeded and UV-irradiated 30, 100, or 300 J/m2 of 254 nm UV.

(iii) RT-PCR and Q-PCR for Quantification of CSB:

Total RNA are isolated using Trizol reagents, and are converted into first strand cDNA by SuperScript III reverse transcriptase (Invitrogen). Mouse CSB primer sequences of sense 5′-GGTAGCCAGCCTGTCTTC-3′ (SEQ ID NO: 8) and antisense 5′-CCTCCTCTTCCTTCCATAGC-3′ (SEQ ID NO: 9) were designed by the Becon Primer Designs software. Q-PCR are performed using Bio-Rad iQ cycler. CT values are calculated and normalized to the level of the housekeeping gene microglobulin. Relative gene expression is calculated according to 2-^{ΔΔ}CT from three independent experiments.

(iv) CHIP Assay/EMSA Assay to Test the Interaction Between CSB Promoter:

Chromatin ChIP is carried out using the Upstate Biotechnology (Charlottesville, Va.) ChIP assay kit with modifications. TR4-transfected cells are lyzed, cross-linked with 1% formaldehyde, and chromatin pellets are sonicated to an average of 200 to 1000-bp fragments of DNA. The chromatin fragments are subjected to immunoprecipitation with TR4 antibody overnight at 40 C. The precipitates are eluted into the elution buffer containing 1% SDS, 100 mM NaHCO3, and 10 mM DTT. The cross-links are reversed with 4 h incubation at 650 C in the elution buffer with addition of 200 mM NaCl. The immunoprecipitated DNA fragments are purified using QIAGEN MiniElute Reaction Cleanup kits and subjected to PCR using a pair of primers which are designed to amplify the mouse and human CSB promoter sequences containing putative direct repeat sequences. Electrophoretic Mobility Shift Assay (EMSA) is performed by incubating the ³²P-end-labeled mouse and human CSB probes with the cell nuclear extracts or in vitro translated protein. For antibody supershift assay, monoclonal antibodies specific for TR4 are incubated with the reactions for 15 min at 25° C. prior to loading on a 5% native gel.

(v) UV-Induced DNA Damage Repair Assay (Transcription-Coupled Repair):

Cells are transfected with 0.5 μg of the SV40-renilla damaged by 5000 J/m2 of UV irradiation and 0.1 μg of the undamaged SV40-lacZ, and transfected with PCMX-TR4 transfected cells or the pCMX in mammalian cells, including CV-1, C2C12, and mouse MEF cells. 24 hours after transfection, luciferase and β-galactosidase assays are performed. DNA repair was assayed by the luciferase activities. SV40-renilla luciferase activities were normalized to that of SV40-lacZ. Fold repair are calculated by dividing by the normalized luciferase activities by that of the empty vector.

(vi) GGR Assay:

Cells are transfected with 0.5 μg of the pBluescript vector (Stratagene) damaged by 5000 J/m2 of UV irradiation, 0.1 μg of the undamaged pGL3-Basic vector (Promega), and 0.4 μg of the pCMX-TR4 construct or 0.4 μg of the PCMX (an empty vector). DNA repair are assayed by quantitative real-time PCR using T3 and T7 primers for the pBluescript vector, and GL2 and RV3 primers for the pGL3-Basic vector. pBluescript PCR quantities are normalized to pGL3-Basic PCR quantities. Fold repair are calculated from the normalized PCR quantities divided by those of the empty vector.

(vii) Rescue the DNA Repair Deficient by Sending Back Gadd45 and CS-B Genes.

TR4 KO MEFs are infected by retroviral p-BabeGadd45a and p-Babe CSB. DNA repair efficiency are assayed by using TCR and GGR assays.

(11) The Roles of TR4 Involved in Double-Strand DNA Breaks Repair to Control Gamma-Irradiation Sensitivity.

Double-strand DNA breaks (DSBs) are mostly caused by ionizing irradiation. It is extremely important for cells to repair this kind of damage as DSBs are susceptible to exonucleases, leading to loss of large genomic regions. Three repair pathways are involved in repairing DSBs: a true repair by homologous recombination (HR), a less accurate repair by non-homologous send jointing (NHEJ), and a transitional pathway between HR and NHEJ: single-strand annealing (SSA). It was found herein that TR4 KO cells are more sensitive to the gamma irradiation than cells from wt mice, and protein extracts from the TR4 KO mice show less capacity in repair DSB by the End-Joint assay. One IR-responsive gene, Gadd45a was significant reduced IR response in TR4 KO cells. Therefore, TR4 facilitates IR-induced DSB repair by inducing gene expression, such as Gadd45a in repairing DSBs and/or participating with the IR-inducing DNA repair protein complex. Herein, the roles of TR4 in modulating the IR sensitivity are determined and the molecular mechanism of how TR4 regulates the DSB-induced repair defined.

(12) To Characterize the Relationship Between DNA Repair, Fidelity, and Radiation Sensitivity, and to Determine if this is a Result of Wild-Type TR4 Expression.

The data indicate increased DSBR gene expression, as well as TR4 protein expression in irradiated cells, which indicates that TR4 is involved in the machinery of a variety of repair processes, including both base excision repair (BER) and nucleotide excision repair (NER). The interaction of TR4 with proteins involved in HRR, DNA-PK and other NHEJ components activates the machinery and wt TR4 allows the up-regulation of proteins involved in double-strand break repair (DSBR) in response to IR. In addition to DSBs repair machinery, multiple repair proteins are involved in repair processes, therefore it is important to know the effects of TR4 on global repair. Therefore, the comet assay are performed on TR4 KO and TR4 wt cells to determine if differences in radiation-sensitivity correlate with decreased DNA repair ability. Fidelity of repair is important to the fate of the cell, as inaccurate repair can lead to mutations and genomic instability that can contribute to carcinogenesis. Chromosomal lesions include gene amplifications, translocations, and aneuploidy. Some of these lesions are the direct result of mechanisms that involve recombinatorial processes (Livingstone L R, et al. 1992 Cell 70:923-35; Greenwald B D, et al. 1992 Cancer Res 52:741-5). Proper DSBR is necessary for the maintenance of the genome and affects survival in response to DNA damaging agents. The data indicate that TR4 is involved in DSBR and radiation sensitivity. Decreased fidelity in repair leads to increased radiation sensitivity as damaged cells undergo cell death. Therefore, fidelity in DSBR is measured to determine if a difference is observed in repair between TR4 KO and TR4 wt cells, contributing to radiation survival.

(a) Detailed Methods:

(i) Protein Analysis.

Proteins are extracted from TR4 KO and TR4 wt MEFs at 0, 3, 6, 9, and 12 h post-9Gy irradiation, and analyzed on SDS-PAGE gels. Protein samples are transferred to nitrocellulose membrane, and immunoblotted with anti-Rad52, -Rad54, -Rad51 ABs, and anti-DNA-PKcs, -Ku70, -Ku86 ABs. Proteins are detected using enhanced chemiluminescence (ECL) (Amersham).

Rad51 cleavage during the induction of apoptosis from a 36 kDa protein to 21kDa fragment in response to IR results in the loss of recombinase activity (Huang Y, et al. 1999 Mol Cell Biol 19:2996-97). Therefore, in order to study the activity of Rad51 in TR4 KO and TR4 wt cells, immunoblot analysis are performed. Protein extracts are generated as previously described, and TR4 KO and wt samples are run on SDS-PAGE gels. Protein are transferred to nitrocellulose, and probed with rabbit anti-Rad51 antibody. Goat anti-rabbit-IgG secondary antibody conjugated to HRP and ECL are used to detect signal. Cleaved Rad51 protein fragments are indicative of loss of Rad51 activity in cell extracts.

(ii) DNA-PK activity.

DNA-PKcs is a 450 kDa protein that has extensive homology to the phosphotidylinositol-3-kinase protein kinase family. This family includes ATM and ATR, in addition to DNA-PK (Yin et al., 1992). It has been shown that DNA-PK activity is reduced during apoptosis (Bharti et al., 1998), it is possible that DNA-PK activity was inhibited in TR4 KO cells therefore affect radiation sensitivity in these cells. To further characterize the repair capacity in these cell lines, the activity of DNA-PK in its ability to phosphorylate known substrate p53 are assessed (Bharti A, et al. 1998 Mol Cell Biol 18:6719-28; Douglas P, et al. 2001 J Biol Chem 276:18992-8) In vitro kinase reactions are performed as previously described (Brown K D, et al. 2000 J Biol Chem 275:6651-6; Kurimasa A, et al. 1999 Mol Cell Biol 19:3877-84). Cells treated with 9 Gy irradiation are collected at 0, 3, 6, 9, 12 hours post-irradiation. Cells are washed with 1×PBS, and lysed in lysis buffer. Protease inhibitors are added to the lysis buffer. The lysate are adjusted for equal protein content and DNA-PKcs antibody are added to the lysate at a concentration of 5.0 μg/mg lysate and incubated for 2 h on ice. 15 μl of a 50% slurry of protein A/G-Sepharose beads (Amersham) are added, and the incubation continues for 1 h on an end-over-end rotor at 4° C. The immune complexes are washed three times with lysis buffer containing 500 mM NaCl and twice with kinase buffer. The beads are suspended in a minimal volume of kinase buffer and used in kinase reactions.

Kinase reactions are carried out in a final reaction volume of 35 μl. 1 μg of bacterially synthesized, purified recombinant GST-p53 protein, 5 μM cold ATP, 30 μCi γP-32 ATP, and 500 ng of sonicated salmon sperm DNA are added to the slurry of beads containing immuno-precipitated DNA-PKcs. This reaction are incubated at room temperature for 30 min and terminated by adding an equal volume of 3×SDS sample buffer, followed by boiling. The final reaction products are resolved on an 8% polyacrylamide gel and the gel are dried on a slab gel drier (Hoefer Scientific Products). The dried gel are exposed to X-ray X-OMAT-AR film (Kodak), and kinase activity are quantified by phosphorimaging.

(iii) Rad51 Co-Localization.

Following exposure to IR, Rad51 foci must be redistributed to the sites of nuclear DNA damage (Tashiro S, et al. 2000 J Cell Biol 150:283-91). Strand breaks leaving 3′ hydroxyl ends can be labeled with bromylated deoxyuridine triphosphate nucleotides (Br-dUTP) by using the terminal deoxynucleotidyl transferase (TdT) enzyme. Double staining with Rad51 allows the direct visualization of cells that have accumulated Rad51 at the site of DNA damage. TR4 KO and TR4 wt cells are fixed in 4% paraformaldehyde at 0, 3, 6, 9, 12, and 24 h post-9Gy IR. Next, nuclei are permeabilized with 1% SDS/0.5% Triton X-100/1×PBS for 10 minutes. For the detection of strand breaks, fixed nuclei are incubated with mouse or rat anti-BrdU antibody diluted in 1.0% BSA/1×PBS. Rabbit anti-Rad51 antibody is mixed to this primary antibody for the simultaneous detection of Rad51. FITC sheep/goat anti-mouse or anti-rabbit, and Cy3-conjugated goat/sheep anti-rabbit or anti-mouse AB are used as secondary antibodies. Fluorescence are detected by confocal microscopy, and FITC and Cy3 double-positive stained cells are analyzed to determine Rad51 activity in TR4 KO and TR4 wt cells.

(iv) Localization of TR4 During the DSB Repair.

To determine if TR4 binds directly at the site of DSB to interacts with HRR machinery, three color microscopy are performed to observe the co-localization of Rad51 and p53 at strand breaks in response to treatment with IR. Cell collection, fixation and staining are performed as described above (Haaf T, et al. 1999 J Cell Biol 144:11-20; Scully R, et al. 1997 Cell 88:265-75) TR4 KO and TR4 wt MEFs or primary dermis-derived fibroblasts are stained with mouse anti-TR4, rabbit anti-Rad51 anti-Rad52 and rat anti-BrdU ABs. Secondary antibodies containing fluorescent conjugates to PE, FITC and Cy3 are available to each primary antibody. Immunofluorescence is recorded using a confocal microscope. In addition DAPI counterstaining are performed following washing with PBS (Haaf T, et al. 1999 J Cell Biol 144:11-20) The colocalization and interaction of TR4 with Rad51 at DSBs are indicative of suppression of HRR.

(v) Comet Assay.

The comet assay, also called single-cell gel electrophoresis assay (SCGE) performed under neutral conditions allows for the detection and kinetics of repair of DNA DSBs (Fairbairn D W, et al 1995 Mutat Res 339:37-59). It has been shown that cell lines deficient in DNA repair exhibit slower rejoining and contain more residual damage (Olive PL 1999 Int J Radiat Biol 75:395-405). In addition, cells that are unable to repair DSBs exhibit increased sensitivity to DNA damaging agents. The neutral comet assay are performed as previously described (Lips J, Kaina B 2001 Carcinogenesis 22:579-85), using the following modifications. After 9 Gy IR treatment, TR4 KO and TR4 wt cells are harvested at 0, 3, 6, 9, 12, 16 and 24 h by trypsinization. Agarose-coated (1.5% in PBS) and dried slides (Trevigen Comet slides) are prepared. 1×103 to 1×104 cells (in 10 μl) are embedded in 120 μl low-melting point agarose on these slides (0.5% in ddH2O at 37° C.). The slides are submersed for 1 h in pre-cooled lysis buffer. One hour before electrophoresis 1 ml Triton X-100 and 10 ml DMSO/100 ml are added. Slides are electrophoresed at 25V for 15 min. After EtOH fixation, drying, and SYBR Gold (Molecular Probes) staining, cells are analyzed by epi-fluorescence microscopy using a FITC filter, and tail moment is determined by measuring the fluorescence intensity using MetaMorph Imaging software (Universal Imaging Corporation). 25 individual cells for each treatment are scored and average length of tail moment and percentage of DNA in the tail moment are determined at each time point. Cells with greater tail moment are scored as having the most residual DNA damage. The induction of DSBs in TR4 KO and TR4 wt cells, as well as the kinetics of repair are determined using this method.

The comet assay can only measure the kinetics of disappearance of smaller fragments of DNA, and therefore repair capacity of a single cell. While small fragments of DNA are obviously broken DNA, larger fragments of DNA are not necessary correctly repaired.

(vi) DSBR Fidelity.

The fidelity assay examines the ability of TR4 KO and TR4 wt MEFs nuclear extracts to rejoin DSBs introduced into the lacZ gene of plasmid DNA, thereby restoring expression of β-galactosidase. β-galactosidase activity can be measured by blue colony formation on X-gal plates.

In order to determine if TR4 has an effect on DSBR fidelity, break rejoining by TR4 KO and TR4 wt nuclear extracts are analyzed. TR4 KO and TR4 wt proteins are extracted as previously described. pUC18 plasmid DNA are isolated and purified using standard procedures (Quiagen). Double-strand breaks are introduced in the multi-cloning region of the plasmid, disrupting the lacZ gene, using HindIII, BamHI, or EcoR1 restriction endonucleases as previously described (North P, et al. J 1990 Nucleic Acids Res 18:6205-10; Thacker J. et al. 1992 Nucleic Acids Res 20:6183-8).

As a control to check cutting efficiency, southern analysis is performed. Approximately 1 ng of plasmid DNA are heated to 65° C. prior to running on a 1% agarose gel (without ethidium bromide) in TBE buffer, blotted to nitrocellulose, and probed with pUC18 labeled by the random oligonucleotide method (Feinberg A P, Vogelstein B 1983 Anal Biochem 132:6-13) to a specific activity of 0.4−2.0×10⁹ cpm/μg DNA.

Plasmid DNA and protein extracts are mixed in 50 μL reactions containing 65.5 mM Tris-HCl, pH 7.5, 10 mM Mg SO₄, 1 mM ATP and 40 μg/ml DNA. The reaction is incubated for 20-26 h at 14° C., and DNA are purified by phenol/chloroform extraction and EtOH precipitation.

Bacterial transformations are carried out in DH5α E. coli. The transformants are selected on LB plates containing ampicillin (100 ug/ml) and X-gal (5-Bromo-4-chloro-3-indolyl-β-D-galactopyranoside, 40 μg/ml). Bacterial viability is assessed on plates containing X-gal but no ampicillin. White colonies (not expressing β-galactosidase) are streaked onto LB plates with ampicillin and X-gal for confirmation. Blue colonies are scored as having fidelity in DSBR. The frequency of mis-rejoining is estimated as the number of white colonies relative to total colonies (blue+white) counted.

(13) Determine if TR4 is Involved in Protein:Protein Interactions with DNA Repair Machinery in Irradiated Cells.

It is disclosed herein that TR4 recognizes DNA lesions, and then binds to DNA through sequence non-specific binding and interacts with factors involved in HRR and NHEJ at the sites of DNA damage thereby activating HRR and/or NHEJ repair, and altering radiation-sensitivity.

(a) Detailed Methods:

(i) Chromatin Fractionation.

It is disclosed herein that TR4 protein is important in regulation of DSBR through sequence non-specific DNA binding at sites of DNA damage. Therefore, chromatin fractionation and immunoblot analysis are performed to determine the role of TR4 in protein:protein interactions with DSBR proteins on chromatin in irradiated cells. These studies address the question of whether TR4 is required for activation of proteins involved in HRR and NHEJ, and help to understand the roles of repair proteins on p53-mediated radiation sensitivity.

(ii) Chromatin Fractionations are Performed.

A total of 3×10⁶ cells are washed with PBS and resuspended in 200 μl solution A (10 mM HEPES, pH 7.9, 10 mM KCl, 1.5 mM MgCl₂, 0.34 M sucrose, 10% glycerol, 1 mM DTT, 10 mM NaF, 1 mM Na₂VO₃, and protease inhibitors). Triton X-100 are added to a final concentration of 0.1%, and the cells are incubated on ice for 5 min. Cytoplasmic proteins are separated from nuclei by low-speed centrifugation (1300 g for 4 min). Isolated nuclei are washed once with solution A and lysed in 200 μl solution B (3 mM EDTA, 0.2 mM EGTA, 1 mM DTT). Following a 10 min incubation on ice, soluble nuclear proteins are separated from chromatin by centrifugation (1700 g for 4 min), washed once with solution B, and spun down at high speed (10,000g for 1 min). Chromatin are resuspended in 200 μL SDS sample buffer and sheared by sonication. To digest chromatin with micrococcal nuclease, nuclei are resuspended in solution A containing 1 mM CaCl₂ and 50 units of micrococcal nuclease (Sigma). Following 2 min incubation at 37° C., nuclei are lysed and fractionated as above. This procedure results in the extraction of chromatin-bound proteins, and the exclusion of unbound proteins.

(iii) Immunoprecipitation and Immunoblot Analysis.

MEFs lysates and immunoprecipitations are prepared as described previously (Pandey A, et al. 1996 J Biol Chem 271:10607-10) following 9Gy IR treatment. 150 μg of soluble protein are incubated with anti-DNA-PK, anti-TR4, or anti-Rad51 for 2 h at 4° C. Immune complexes are precipitated with Protein A-sepharose beads for an additional 2 h at 4° C. After five washes with lysis buffer, the immunoprecipitates are resolved by SDS-PAGE, and transferred to nitrocellulose. The residual binding sites are blocked with 4% skim milk in TBST (tris-buffered saline, 0.1% Tween-20). The filters are immunoblotted with anti-Ku70, -Ku80, -DNA-PK, -Rad51, -Rad52, -Rad54 and -TR4. After washing with TBST, the blots are re-blocked and incubated with secondary anti-mouse or anti-goat IgG HRP conjugate. The antibody complexes are visualized by enhanced chemiluminescence. The data show which proteins are specifically bound to DNA in TR4 KO and TR4 wt cells. This helps determine which complex formations are activated/repressed preferentially by TR4.

(14) To Determine which Domain of TR4 is Primarily Responsible for the Increased Radiation Sensitivity Observed in TR4 KO MEFs.

TR4 protein has many effects in the cell; in addition to binding specifically to DNA at TR4 consensus sites (mainly in P-box in DNA binding domain), TR4 binds non-specifically to a variety of substrates, including ssDNA, DNA duplex with free ends, nicked DNA, DNA damaged by IR, and DNA with Holiday junctions. It is disclosed herein that TR4 has duel functions following IR exposure: through sequence-specific binding and transcriptional activation of genes involved in cell-cycle regulation and apoptosis mediated through its central domain, and through sequence non-specific interaction with broken DNA. Which domains of TR4 can bind is determined with DNA and allowed subsequent interactions with members of the HRR machinery. Different domain deletions of TR4 mutants are tested. Therefore, wt TR4 full length, three truncated cDNA vectors (TR4-ΔN, TR4-4A4, and TR4-ΔC) are constructed into the retroviral vector and then transiently transfected into TR4 KO cells to determine the involvement of the various TR4 constructs in radiation sensitivity by the IR-cell survival assays. To determine which portion of TR4 is important for DNA repair not only provide molecular information regarding the actions of TR4, but also a useful tool for future design to altering the IR sensitivity.

(a) Detailed Methods:

(i) Construction of Truncated cDNA TR4 to Retroviral vector:

Three TR4 domain mutants, pCMX-TR4-ΔN(N-terminal deletion) (Shyr C R, et al. 2002 J Biol Chem 277:14622-8), pCMX-TR4-4A4 (replaced TR4 DNA binding domain with androgen receptor DBD) (Lee Y F, et al. 1998 J Biol Chem 273:13437-43) and pCMX-TR4-ΔC(C-terminal deletion mutant) (Shyr C R, et al. 2002 J Biol Chem 277:14622-8) were described previously. These TR4 mutants are re-constructed into pBabe vector, and tested for their ability to rescue TR4 KO.

(ii) Retroviral-Mediated Gene Transfer:

To restore TR4 expression in TR4 KO MEF cell, pBabe-hTR4, and pBabe-TR4-ΔN, pBabe-TR4-4A4, pBabe-TR4-ΔC is used for retroviral infection. Ecotropic packaging cells are plated for 24 h and then transfected with SuperFect (Quigen) with pBabe-pur/2 or pBabe-TR4. After 48 h the viral containing medium are filtered (0.45 mM filter, Millipore) to obtain viral-containing supernatant. Targeted MEF cells are plated and the culture medium is replaced with a mix of the viral-containing supernatant and culture medium, supplemented with 4 μg/ml polybrene, and the cells are incubated at 37° C. MEF cells infected with the empty vector (pBabe-puro) are used as control.

(15) Determination of Roles of TR4 in Regulation of Irradiation Sensitivity, Both In Vitro and In Vivo.

As shown herein, TR4 KO MEFs are more sensitive to irradiation, indicating that TR4 is a candidate molecule to control the cell response to irradiation. Therefore, TR4 serves as an irradiation sensor by altering the cell cycle signals, and participating in DBS DNA repair to prevent the irradiation-induced cell death, and decreasing TR4 activity reverses these effects. Many cancer cells such as lung, brain, and prostate are radiation resistant, in which cancer cells have developed ways to escape from radiation-induced cell apoptosis. Thus, by modulating TR4 expression/activity in these cancer cells, the cells are rendered more sensitive to irradiation. In addition, TR4 is a member of nuclear receptor, and it possibly can be activated/modulated by their ligand/agonist/antagonist, therefore small molecules can be used to modulate TR4 activity allow the cancer cells are more susceptible to radiation.

(16) Determination of Effects of Alteration of TR4 on Radiation Sensitivity in the Cancer Cells

(a) Examine the TR4 Status in Cancer Cells and Correlate to their Radiation Sensitivity.

Several cancer cell lines, such as human lung cancer A549 (p53 wt) and H1299 (p53-null) cells, rat 9 L brain tumor cells, and human prostate cancer LNCaP, DU145, and PC3 cells, are used. Endogenous TR4 levels as well as post-IR stimulated TR4 are examined. TR4 mRNA level changes are quantified by Q-PCR and protein expression level/patterns are examined by Western blot analysis by plotting with antibodies against TR4 and phospho-TR4 that are identified by previously. Cells are exposed to different doses of gamma radiation, from 0, 3, 6, 9, and 15 Gys and then cells are counted at day 0, 2, 4, and 6 by MTT assay and ³H-thymidine incorporation assay. IR sensitivity is calculated as the ratio of the surviving cells of post-IR to the surviving cells without exposure to IR. The correlation of TR4 level with IR sensitivity are compared.

(b) Changing the IR Sensitivity by Engineering Altered TR4 Activity in the Cells.

Lower TR4 activity in the cells can result in enhancement of IR sensitivity. Herein, whether changing the TR4 amount in the cells changes the IR sensitivity is examined. Therefore, TR4 expression is overexpressed and knocked down in the cells. The cells with different amounts of TR4 are exposed to irradiation and cell survival is measured. Basically, the viral gene transfer techniques are applied to deliver either TR4 cDNA or TR4 RNAi into the cells and select at least three clones with altering TR4 expression levels to test IR sensitivity. Thus, TR4 is a key factor to control the IR sensitivity, and if altering the TR4 inside the cell indeed alters the IR sensitivity.

(c) Changing the IR Sensitivity by Modulation of TR4 Activity Through Controlling its Upstream Signals.

As previously shown, IR changes TR4 protein phosphorylation status. Results from phosphor-TR4 mutant studies, indicated that phosphorylation of ser-351, a potential AMPK and 14-3-3 phosphorylation site, are important for TR4 activity. Therefore, the kinase-cascade signal is utilized to control TR4 activity, and then examine IR sensitivity changes after TR4 activity is altered. This is proof that the signal cascade that affects TR4 activity can be altered to achieve a change of cell IR sensitivity. When TR4 activity correlates with radiation sensitivity, then TR4 status, including expression levels and phosphorylation status can be very good markers to predict the radiation sensitivity. Targeting reduced TR4 activity in the cancer cell is a potential radiosensitizing strategy in radiotherapy.

(d) Detailed Methods:

(i) Determination of TR4 Expression by RT-PCR, Q-PCR, Western Blotting Analysis:

RNA and protein from cancer cell lines are extracted as described in the results, and are subjected into semi-quantitative PCR, Q-PCR, and Western Blotting analysis. 12.5% to 15% SDS-PACE are applied to distinguish the different phosphorylation forms of TR4.

(ii) Determination of IR Sensitivity:

Cancer cells are exposed to different dose of IR (0, 3, 6, 9, and 15 Gys) and harvested at day 0 (without IR), day 2, 4, and 6. Cell proliferation rate are determined by MTT, and 3H-thymidine incorporation assay.

(iii) Overexpression or Knockdown Endogenous TR4 by Retroviral-Mediated Gene Transfer:

As mentioned before, TR4 overexpression (by pBabe-TR4) and knockdown (by TR4 RNAi) systems were established, and can be stabilized into the cancer cells by antibiotic selection, and confirm TR4 expression by Q-PCR, and Western Blotting analysis.

(iv) Altering TR4 Activity by its Upstream Signals.

As shown herein, mutants of TR4 at the AMP kinase site (Ser-351) result in altering TR4-mediated transactivation potential (TR4 S351A: gain-of-function, and TR4 S351E: lost-of-function), therefore, the upstream AMP kinase activator (AICA-Riboside: 0.5-2 μM, CalBiochem), and AMP kinase inhibitor (AMPK inhibitor compound C: 5-40 μM, CalBiochem) are applied to MEFs for 12 hrs before IR treatment, and then examine the post-IR response. In addition to AMP kinase pathway, other compounds identified herein are applied to control the signals which can affect TR4 activities.

(17) Compare the IR Sensitivity in the TR4 KO Vs Wt Mice.

As shown herein, MEFs derived from TR4 KO mice are more sensitivity to IR than MEFs derived from wt mice; therefore, it is likely that TR4 KO mice are more sensitive to the gamma radiation. Herein, the survival rate in TR4 KO mice vs wt mice is examined after they are exposed to IR.

(a) Detailed Methods:

Ten pairs of 8-week-old TR4 KO mice and wt littermates are exposed to 2.5 and 5.0 Gy total body dose of gamma irradiation, and animal survival are monitored and recorded. The mice are sacrificed and the internal organs (skin, liver, prostate, spleen, and bone marrow) are preserved for further IHC analysis of DNA repair genes expression profiles comparison.

(18) Comparing the IR Sensitivity in the Nude Mice that were Baring Tumor with Amount of TR4.

The data indicated that TR4 is involved in IR induced DNA-repair, and cells containing TR4 are more resistant to IR than the cells without TR4. To apply this concept in the cancer therapy, it is important to determine if TR4 also regulates in vivo radiation sensitivity xenograft tumor model. Therefore, a human tumor cell line xenograft model is generated to compare the radiation sensitivity in the nude mice bearing tumor with different amount of TR4. The IR sensitivity is correlated with the amount of TR4 to test if TR4 is a mediator for IR-induced tumor cell apoptosis.

(a) Approaches:

Cancer cells with overexpressed and knockdown endogenous TR4 are implanted into the nude mice. Once the tumors grow, the mice are then be treated with gamma radiation (2.5 and 5.0 Gy total body dose). The tumor size is reduced in response to IR. The different response to IR between the mice bearing different amounts of TR4 are monitored and recorded as an indication for tumor radiosensitivity.

(b) Detailed Methods:

(i) Human Cancer Xenograft in an Athynic Mouse model:

The xenograft model, which mimics cancer progression in vivo, is used herein. Nude male mice, are maintained for 2-4 weeks prior to the tumor studies, and housed under normal lighting. Young adult male mice, at age of 8-10 weeks are subcutaneously injected with 3×10⁶ cancer cells with different amount of TR4. Tumors are allowed to grow, measured weekly with calipers, and tumor volumes are calculated using the formula 0.532×r1²×r2 (r1<r2). Once tumors reach a volume of 0.4 cm³, cancer-bearing animals are randomly grouped in three categories: Little J B1994 Radiat Res 140:299-311 without IR, Takata M, et al. 1998 Embo J 17:5497-508 IR (2.5 Gy total body dose), and Salles-Passador I, et al. 1999 C R Acad Sci III 322:113-20 IR (5.0 Gy total body dose) twice per week for 6-8 weeks. Animals are weighed, and tumor size is measured tree times per week to monitor the IR effects and toxicity induced by IR. In all animals, once tumor volumes are observed to reach to 10% of body weight, animals are sacrificed; otherwise the animals are sacrificed at the end of 8-week treatment. Tumor-bearing animals from all groups are sacrificed by cervical dislocation and blood is collected. Tumors are excised, weighed, and half of the tumor is stored in liquid nitrogen for later analysis. The other half of the tumor are fixed and embedded for immunohistochemical analysis. The prostate gland, lung, lymph nodes, and bone marrow are examined for tumor metastases. Ten animals per group are analyzed. The IR-responsive genes (p53, p21, p16, p19, Gadd45, ATM, Rad51, and CS-B) are measured by Q-PCR and Western Blotting, and TR4 expression and its phosphorylation status are determined by Western Blotting analysis.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Example 4

### Aim 1

### To Elucidate the Underlying Mechanisms of How TR4 Guards the Genomic Stability to Suppress Tumorigenesis, and to Identify the Molecules which are Responsible

Deletion of TR4 increases the genomic instability and results in accelerated aging in mice and an early onset cell G2/M arrest. Aging mice with TR4 deleted develop prostatic hyperplasia and/or displasia. TR4 is aberrantly expressed in human prostate cancer. In combination with mice and clinical studies, it is disclosed herein that as a transcriptional factor, TR4 is a caretaker gene which maintains genome stability to suppress prostate carcinogenesis via transcriptional regulating genes involving in cancer progression. To determine TR4 effects on prostate carcinogenesis TR4 expression is modulated in human prostate cells by overexpression and knockdown of TR4 and compare cell behavior in different contents of TR4, followed by gene profiling to identify TR4 regulation genes using the human cancer-pathway focus oligo-GenArray. Identified genes can be further confirmed for their roles in mediating TR4-regulatory prostate carcinogenesis pathway.

a) Study the Effects of Suppression and Overexpression of TR4 in Prostate Cells on Genome Stability and Prostate Cancer Cell Tumorigenecity.

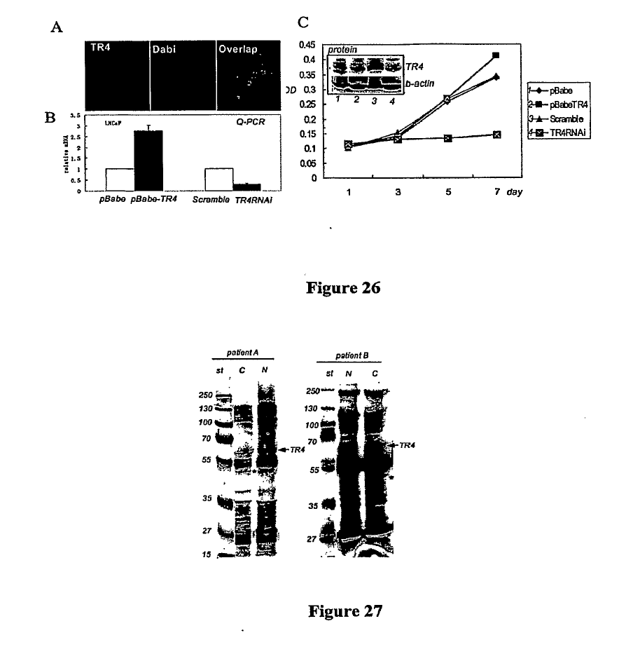

To test if TR4 is directly involved in prostate carcinogenesis as well as to understand the molecular mechanism by which TR4 regulates PCa behavior, prostate cells with alteration of TR4 expression are established by targeting overexpression and/or knockdown of TR4 gene and then examine the prostate cancer cell behavior. LNCaP cells were chosen because TR4 locates in the nuclear (FIG. 26A) and there is no mutation identified from TR4 cDNA. As shown in FIG. 26B, TR4 mRNA expression level is significantly reduced and increased in TR4 RNAi and pBabe-TR4 transfected cells by Q-PCR analysis. Interestingly, knocking down TR4 in LNCaP cells suppressed LNCaP cells growth; reversely, overexpression of TR4 slightly induced LNCaP cell growth at day 7 (FIG. 26C). To confirm the effects of TR4 on LNCaP cell growth, more clones are selected and analyzed for growth, cell cycle profile, tumor invasion potential and in vitro tumorigenesis. Similar approaches can be applied to PC-3 cells and non-transformed prostate epithelial BPH-1 cells. When modulation of TR4 indeed alters prostate cell growth and tumorigenesis in vitro, the xenograft model, a standard in vivo tumorigenesis assay, is applied.

b) Identify and Verify the Molecules that are Responsible for TR4 Anti-Tumor Action Using Human Cancer Pathway Focus Oligo GenArray.

To examine the molecular mechanisms by which TR4 controls the prostate tumor progression, the DNA microarray is applied to compare the gene profiles between TR4 RNAi and scramble control. To be more specific and focus on TR4 roles in cancer, an Oligo GEArray Human Cancer Microarray (SuperArray) is used to screen the TR4 target genes. This microarray profiles the expression of 440 genes that include members of six pathways frequently altered during the progression of cancer. First, LNCaP-TR4 RNAi and -scramble control are compared. The genes that show differential expression (>2 fold changes) are confirmed further by Q-PCR and Western. The cancer pathway in this microarray is pre-determined, so specific pathway(s) that are controlled by TR4 can be determined. When other TR4 stable prostate cell clones become available, those TR4-modulated stable clones are screened to confirm the presence of a universal pathway controlled by TR4. To confirm those genes are transcriptionally regulated by TR4, a promoter reporter study is conducted. The promoter region of the target gene are PCR amplified and cloned into pGL3-basic that is available in the lab. The effects of TR4 on the reporter activity is examined. When the results are positive, the TR4REs in the reporter are mutated to verify and applied the DNA pull-down and ChIP assay to test direct binding.

c) Confirmation of the Roles of Those Identified Molecules in TR4-Mediated Anti-Tumor Pathway.

The identified TR4 targeted genes are examined for their influences on TR4-mediated anti-tumor pathways by modulating their expression through knockdown and overexpression. To confirm the roles of TR4 positive regulated target genes in the anti-tumor pathway, TR4 activity is “rescued” by sending the TR4-positive regulated genes back to the TR4 deficient cells and examine if this restores TR4 anti-tumor activity; in contrast, for TR4 negative regulated genes, knocking down target genes should be able to restore TR4 activity in TR4 deficiency cells. Identified TR4 targeted genes can be cloned by RT-PCR into mammalian vector, and lentivirus vector for transfection, and functional rescue experiments are performed as follows.

d) Detailed Methods:

(1) Establishment of TR4RNAi-, and Overexpression of TR4 in PC-3 and BPH-1 Cells.

Retrovirus infection is used to deliver TR4 RNAi and pBabe-TR4 and their vector control to cells. Cells are selected and TR4 expression levels are confirmed by Q-PCR and Western blotting.

(2) In Vivo Tumorigenesis Assay (Nude Mice Xenograft Model):

Nude male mice are maintained for 2-4 weeks prior to the tumor studies, and housed under normal lighting. Young nude mice (6-8 weeks old) are injected subcutaneously into the dorsal flap with 2−3×10⁶ prostate epithelial cells. Tumors are allowed to grow, measured three times every week with calipers, and tumor volumes are calculated using the formula 0.532×r1²×r2 (r1<r2). In all animals, once increased tumor volumes observed to have reached 10% of body weight, animals are sacrificed; otherwise animals are sacrificed at the end of 12 weeks after cell implantation and blood collection. Tumors are excised, weighed, and half of the tumor is stored in liquid nitrogen for later analysis. The other half of the tumor is fixed and embedded for immunohistochemical analysis. The prostate gland, lung, and lymph nodes are examined for tumor metastases. To achieve the statistic significance, twelve animals per group are used.

(3) Human Cancer Microarray (SuperArray).

To identify TR4 downstream target genes in the cancer pathways, the stable clones that express different amounts of TR4 (pBabe-TR4 vs pBabe; and RNAi-TR4 vs scramble) are compared. The microarray is performed following the user manual. Briefly, total RNA is extracted using ArrayGrade total RNA isolation kit, and RNA quality are monitored by A₂₆₀:A₂₈₀ ratio greater than 2. Total RNA is then converted into cDNA. The cRNA is synthesized and biotin labeled, and purified for hybridization. Signals are developed by utilizing the Chemiluminescent Detect kit, and images captured and analyzed by VersaDoc Imaging system.

(4) Confirmation of the TR4 Target Gene Function:

TR4-down stream target genes are confirmed by altering its expression in the cells (via lentivirus infection of targeted RNAi and overexpression), and then examine the consequence. The methods were described previously.

Promoter studies, DNA-pull down assay and CHIP assay are followed as previously described (Bao B Y, et al. (2006) Carcinogenesis 27:1883-1893).

### Example 5

### Determining the Abnormalities of TR4 in Prostate Cancer and Pathway(s) that Control Subcellular Localization and Tumor Suppression

Prostate TMA analysis of TR4 protein abundance in clinical prostate specimens reveals numerous abnormalities. First, TR4 expression was significantly increased in PIN, LG, and HG as compared to normal prostate specimens. Second, TR4 signals shifted from the nucleus to cytoplasm proportionally with the progression of disease. As a transcriptional factor, nuclear localization of TR4 is essential for its function; therefore, the cytoplasmic TR4, in essence, is not transcriptionally activated. Based on these data, in the early stages of prostate carcinogenesis, TR4 is activated and functional; however, during prostate cancer progression, TR4 is retained in the cytoplasm and loses its functions. The abnormal TR4 protein behavior in the prostate tumor can be due to TR4 gene mutation or alteration of protein character by tumor environment.

a) Proteomic Analyses of the TR4 from High Grade Prostate Cancer Specimen.

Epigenetic protein modifications are known to regulate protein functions and play important roles in multiple processes including DNA repair, protein stability, nuclear translocation, protein-protein interactions, cellular proliferation, differentiation, and apoptosis (Pawson T (1995) Nature 373:573-580; Kouzarides T (2000) Embo J 19:1176-1179; McBride A B, Silver P A (2001) Cell 106:5-8). To investigate if there are any epigenetic modifications of TR4 protein in the high-grade prostate cancer (PCa) specimens, TR4 and TR4-associated complex are immunoprecipitated (P) from the human prostate cancer specimens (C) and their counterpart normal prostate (N) for comparison, by anti-TR4 antibody (#15), as shown in FIG. 27. TR4 protein was pulled down from both cancer and normal prostate specimens with molecular weight around 64 kb (arrow). Interestingly, the 50 kb protein (*) TR4 associated protein (TR4AP-PCa) is only found in cancer specimens but not in the normal counterpart from the same patient. Therefore, TR4 protein and TR4AP-PCa are characterized from the cancer specimens by proteomic analyses (Proteomics Center, University of Rochester). TR4 and TR4AP-PCa is eluted from the gel and subjected into mass spectrometer to identify TR4AP-CaP, and to characterize TR4 protein isolated from prostate cancer (TR4-CaP) in order to determine if there are any abnormalities and/or post-translational modifications, such as phosphorylation, acetylation, oxidation, and nitrosylation, on TR4 protein. To compare cytoplasmic TR4 to nuclear TR4 protein, prostate protein extracts are fractionized into nuclear and cytoplasmic portions and then IP by TR4 antibody for proteomic analyses. More patients' samples can be collected to confirm the results obtained from the first two pairs of sample analyses.

Via proteomic analyses, (1) TR4-PCa protein profile, and (2) TR4AP-PCa identity are revealed. When TR4-PCa is found to be post-translationally modified, the function of TR4-PCa is examined by modulating the post-translational signals. The interaction of TR4AP-PCa with TR4 in the prostate tissue and the consequence of their interaction that affects prostate cancer progression is examined by overexpression and knockdown the TR4AP-PCa via lentivirus infection as well as interruption the interaction between TR4AP-PCa and TRaPCa via interacting peptides. The TR4 functional assays are (1) transcriptional activity, (2) anti-ROS activity, (3) DNA damage repair activity. To measure cancer progression, cell growth/apoptosis, and in vitro tumorigenesity is performed.

b) Determine if TR4 is Mutated in Human Prostate Cancer Tissues and Cell Lines:

Another possibility for TR4 abnormal behavior observed in prostate cancer TMA might be due to TR4 gene mutation. TR4 cDNA was cloned and sequenced from LNCaP prostate cancer cells and BPH-1 non-transformed prostate epithelial cells, and found no mutation. Considering the heterogeneous nature of prostate cancer, genomic DNA is extracted from prostate specimens as well as prostate cancer cell lines, and sequence TR4 exons. TR4 (NR2C2; NM_{—}003298) is located in chromosome 3(3p24.3) and composed of 14 exons, one nuclear localization signal (NLS) site (aa 157-175), and several phosphorylation sites (¹⁴⁴S, ¹⁵⁰S for PKC and PKA; ³⁵¹S for AMPK and 14-3-3) where mutation might occur. TR4 exon fragments are cloned and sequenced for any mutation. Four commonly used prostate cancer cell lines: LAPC4, CWR22rv-1, DU145, and PC-3, and two non-transformed BPH1 and RWPE-1 prostate epithelial cells, where TR4 expresses in both nucleus and cytoplasm is used in this study to verify TR4 gene mutations. When any mutation is identified, the TR4 mutation behavior is further characterized as well as its role in cancer progression. Briefly, the mutated TR4 are introduced into prostate cells (cancer and non-transformed) by retrovirus and then tested for its effects on (1) TR4 transcriptional activity, (2) anti-ROS, (3) DNA repair, (4) cell growth and (5) in vitro tumorigenesity.

c) Determine the Pathway(s) in Prostate Cancer that Lead to TR4 Retention in Cytoplasm.

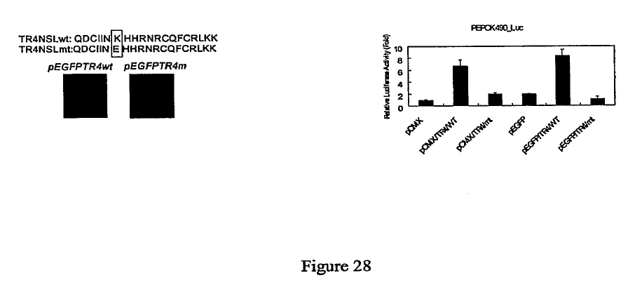

The abnormality of TR4-PCa is determined to be due to gene mutation and/or protein post-translational modification by the tumor environment factors. The TR4-. PCa subcellular localization, how the post-translational modification signals affect TR4 subcellular localization, and how this nuclear-cytoplasmic shuttling abnormality of TR4 contributes to cancer progression are determined. To facilitate the study, the EGFP-TR4 wt, and EGFP-TR4NLS mutant were constructed. As shown in FIG. 28, EGFP-TR4 expresses in the nucleus and transactivated the TR4-responsive reporter (PEPCK-LUC) to the same degree as pCMX-TR4, while EGFP-TR4NLSm retains in the cytoplasm and lost the transactivation potential.

When any TR4 mutants are identified, they are constructed into EGFP vector to examine localization. Or, when any post-translational modification in the cancer environment affects TR4 behavior, EGFP-TR4 can be used to confirm. Briefly, EGFP-TR4 is transfected into the prostate cancer cells to examine its localization by 1) modulation of post-translational signal pathways; and 2) overexpression/knockdown of TR4AP-PCa. Knowing which factor(s) contributes to cytoplasmic retention of TR4 in the tumor, allows for testing how nuclear-cytoplasmic shuttling abnormality of TR4 in prostate cancer contributes to prostate cancer progression by interfering with TR4 nuclear transport, and then examining how this cytoplasmic TR4-PCa affects the tumor progression, such as cell growth, cell invasion, and tumorigenesis.

d) Detailed methods:

(1) Protein Extraction and Fractionalization:

Around 200-500 mg of prostate tissue are homogenized and resuspended in 400 μl cold buffer A (10 mM HEPES-KOH/pH 7.9 at 4° C., 1.5 mm MgCl₂, 10 mM KCl, 0.5 mM dithiothreitol, 0.2 mM PMSF) by flicking the tube allowing cells to swell on ice for 10 min. Samples are centrifuged for 10 sec and supernatant are collected for cytoplasmic faction. The pellets are suspended in 100 μl of cold Buffer C (20 mM HEPES-KOH/pH7.9, 25% glycerol, 420 mM NaCl, 1.5 mMMgCl2, 0.2 mM EDTA, 0.5 mM dithiothreitol, 0.2 mM PMSF) and incubated for 20 min, cell debris are removed by centrifugation for 2 min, and supernatant is collected as nuclear fraction.

(2) Immunoprecipitation of TR4 and TR4-Associated Complex.

Anti-TR4 antibody (#15) is added into protein extracts (cytoplasmic and nuclear), 1/100 dilution at 4° C. for overnight. The complex is pulled down by adding protein A beads and washed three times with PBS. The protein complex profile is analyzed by silver staining or Western blotting.

(3) Mass Spectrometry-Based Proteomic Analysis:

TR4-IP complex is resolved by SDS-PAGE. Gel slices that contain TR4AP-PCa and TR4 are subjected to overnight tryptic digestion and analyzed by MALDI-TOF MS (Applied Biosystems) in the Proteomics Center at University of Rochester. The information dependent acquisition (IDA) can be used to acquire MS and the data searched at MASCOT.

(4) Lentiviral Vectors Construction and Lentivirus Infection.

In order to study TR4AP-PCa effects on prostate cancer progression, lentiviral vectors with overexpressed and knocked down TR4AP-PCa are produced. TR4AP-PCa cDNA is cloned from prostate cancer cell lines by RT-PCR. The lentirival vectors, pWPI for carrying TR4AP-PCa cDNA, and pLVTHM, for carrying TR4AP-PCaRNAi for lentivirus transduction are available in the lab. The cloning strategies, and lentiviral vector producing protocol are described in the Trono lab website. Briefly, viral vector are cotransfected with psPAX2 and pMD2.G into 293T cells, infected into the targeted cells, GFP positive cells are collected by cell sorter.

(5) In Vitro Tumorigenicity (Colony Forming Assay):

Anchorage-dependent and -independent colony forming assays are applied to characterize the tumorigenicity of cells. In anchorage-dependent colony forming assays, cells are seeded in a density at 200 cells/100 mm dish. Medium is refreshed twice per week for three weeks. The plates are stained with crystal violet in methanol, and colonies containing more than 50 cells are counted. In anchorage-independent colony forming assay, treated cells are suspended in a density of 2000 cells/ml 0.4% low melting agarose in 10% FBS/RPMI and plated on top of 1 ml underlayer of a 0.8% agarose in the same medium in 6-well culture plates. Plates are fed twice per week and colonies larger than 50 cells stained with p-iodonitrotetrazolium violet and counted after three weeks.

TR4 function assay, including the anti-ROS and SSDNA damage and cell growth/apoptosis follows the protocols described in preliminary data and publications.

### Example 6

### Correlation of TR4 Expression Levels/Patterns with Clinical Outcomes in Prostate Cancer Patients

PSA has been extensively used as a biomarker for diagnosis and prognosis evaluation for prostate cancer. The clinical significance of elevated PSA values is still debated. Therefore, markers that predict which patients with early stage cancer can survive longer without additional therapy and markers that predict resistance to therapies is of considerable clinical benefit. TMA analysis of TR4 protein expression in prostate cancer reveals a strong correlation of TR4 amounts with prostate cancer aggressiveness, and TR4 expression shifted dramatically from nucleus to cytoplasm with the grade progression of the disease. In addition, TR4 promotes the transcriptional couple repair (TCR) pathway, one of the NER pathways, which suggests it has a role in both cancer susceptibility and drug resistance (Gazdar AF (2007) N Engl J Med 356:771-773; Zheng Z, et al. (2007) N Engl J Med 356:800-808). Thus, TR4 amount and expression profiles, nuclear vs cytoplasm, are a new diagnosis marker for predicting disease behavior, and prognosis marker for predicting patients' outcomes after treatment.

a) Determine if the Expression of Nuclear TR4 is Correlated with the Molecules that are Responsible for TR4 Action.

To more precisely evaluate TR4 role as a new biomarker for prostate cancer, the downstream TR4 target genes, such as CS-B, and new identified genes, are evaluated for their correlation with TR4 expression in TMA. Given that there is no such thing as a single magic marker for any cancer, it is therefore important to examine the correlation of TR4 expression with TR4-regulated genes. The correlation of TR4 with its target genes serves two purposes: 1) confirmation of the results from human prostate cell line studies in clinical human prostate cancer patients; 2) increasing the accuracy in prediction of patients clinical outcomes by combination of TR4 and TR4-regulated genes as panels of biomarkers.

b) Determine if TR4 Expression/Location in Tumors Correlates with Gleason Grade, Tumor Stage, PSA, Low, Intermediate, and High Risk (D'Amico Classification).

The correlation of TR4 expression profiles are examined, including the total TR4, and ratio of nucleus and cytoplasmic (N/C) as well as TR4 target genes with the clinical and pathological features, such as PSA level, age of onset, clinical stage, biopsy Gleason score, and other pathological parameters such as capsular penetration, seminal vesicle invasion or lymph node involvement, and tumor volume. Follow-up data for overall survival, disease-free survival, and tumor recurrence is correlated with TR4 status. Each feature is correlated with TR4 and its target gene expression profiles independently and/or in combination.

c) Determine if TR4 Expression/Location as Well as TR4-Regulated Protein(s) in Tumors Correlate with or Predict Outcome To Therapy, Including Radiation and Androgen Deprivation Therapy.

As therapy has yet to demonstrate a definitive survival advantage, the need for more therapy options and prediction of treatment outcomes is obvious. As shown in studies disclosed herein, TR4 is involved in the NER pathway. It is known that the NER pathway is involved in the resistance of several types of tumors to certain drugs; therefore, TR4 expression and its activity likely affects the treatment outcomes, and can serve as a novel biomarker to predicting patients' outcomes after radiation and androgen depletion (ADT) therapies. Herein, TR4 and its target gene's expression profiles are determined from the patients who underwent radiation and ADT therapies, and correlate with the patients' treatment outcomes. If samples are available, TR4 expression profiles can be analyzed from the same patients' biopsy samples before and after treatments.

d) Detailed Methods:

(1) Building of Diagnosis Prostate TMA:

Formalin-fixed paraffin embedded (FFPE) tissue blocks from prostatectomy and transurethral resection of the prostate (TURP) specimens containing prostate carcinoma can be utilized to construct a TMA (see supporting document for the details). Briefly, TMA consists of paired tissues from the normal, PIN and carcinoma areas of each specimen. Cases can be selected, and patients' clinical data is abstracted from both the pathology report and the medical record. This includes demographic data (age, ethnicity), staging data (tumor size, tumor location, extracapsular extension, metastasis, etc.), clinical data (symptoms, smoking history, serum PSA, clinical stage, previous treatment, etc.) and pathologic data (histologic type, pre-malignant lesions, etc).

(2) IHC of Target Genes on Diagnosis TMA:

IHC is performed on those TMA slides stained with antibodies against those identified TR4 target genes and following the same protocols as described previously herein.

(3) To Examine if TR4 can be a Prognostic Marker on PSA Recurrences after Radical Prostatectomy.

Tumor recurrence after radical prostatectomy, and 5-year survival rate are used to study the patients outcomes to determine if TR4 expression can influence the risk of PSA failure, and the PSA doubling time after radical prostatectomy. The significance of TR4 expression, in combination with its target genes as predictors for PSA recurrence-free survival time can be determined using the Kaplan-Meier analysis and log-rank test. In addition to multivariate analysis, multiple Cox proportional hazard regression models are used to determine whether TR4 is an independent predictor of time to PSA recurrence in the presence of other pathological and clinical markers.

(4) To Examine TR4 Expression Associated with Prostate Cancer Progression and Duration of Patients Who Underwent ADT Treatment.

The correlation of TR4 expression (total and cytoplasmic TR4) with the disease progression cam be examined from hormonally responsive to refractory stage. Androgen-independent cancer is defined as tumors from patients whose disease showed little clinical response to androgen deprivation therapy or who experienced PSA progression after an initial response. An increase in PSA level, clinically palpable recurrence, or the development of additional bone metastasis (or growth of a measurable metastasis) in the presence of castrate levels of serum testosterone, is considered evidence of disease progression.

### 7. K. REFERENCES

Bao B Y, Yao J, Lee Y F 2006 1alpha, 25-dihydroxyvitamin D3 suppresses interleukin-8-mediated prostate cancer cell angiogenesis. Carcinogenesis 27:1883-1893
Bao, B. Y., Hu, Y. C., Ting, H. J., and Lee, Y. F. Androgen signaling is required for the vitamin D-mediated growth inhibition in human prostate cancer cells. Oncogene, 23: 3350 3360, 2004.
Bartkova J, Horejsi Z, Koed K, et al. 2005 DNA damage response as a candidate anti-cancer barrier in early human tumorigenesis. Nature 434:864-70
Bavik C, Coleman I, Dean J P, Knudsen B, Plymate S, Nelson PS 2006 The gene expression program of prostate fibroblast senescence modulates neoplastic epithelial cell proliferation through paracrine mechanisms. Cancer Res 66:794-802
Ben-Porath I, Weinberg R A 2004 When cells get stressed: an integrative view of cellular senescence. J Clin Invest 113:8-13
Ben-Porath, I. and Weinberg, R. A. The signals and pathways activating cellular senescence. Int J Biochem Cell Biol, 37: 961-976, 2005.
Bertoni-Freddari C, Fattoretti P, Casoli T, Spagna C, Meier-Ruge W 1994 Morphological alterations of synaptic mitochondria during aging. The effect of Hydergine treatment. Ann N Y Acad Sci 717:137-49
Bharti, A., Kraeft, S. K., Gounder, M., Pandey, P., Jin, S., Yuan, Z. M., Lees-Miller, S. P., Weichselbaum, R., Weaver, D., Chen, L. B., Kufe, D., and Kharbanda, S. Inactivation of DNA-dependent protein kinase by protein kinase Cdelta: implications for apoptosis. Mol Cell Biol, 18: 6719-6728, 1998.
Bill C A, Yu Y, Miselis N R, Little J B, Nickoloff J A 1997 A role for p53 in DNA end rejoining by human cell extracts. Mutat Res 385:21-9
Bohr, V. A., Brosh, R. M., Jr., von Kobbe, C., Opresko, P., and Karmakar, P. Pathways defective in the human premature aging disease Werner syndrome. Biogerontology, 3: 89-94, 2002.
Bohr, V. A., Stevnsner, T., and de Souza-Pinto, N. C. Mitochondrial DNA repair of oxidative damage in mammalian cells. Gene, 286: 127-134, 2002.
Braybrooke J P, Spink K G, Thacker J, Hickson ID 2000 The RAD51 family member, RAD51L3, is a DNA-stimulated ATPase that forms a complex with XRCC2. J Biol Chem 275:29100-6
Brown, K. D., Lataxes, T. A., Shangary, S., Mannino, J. L., Giardina, J. F., Chen, J., and Baskaran, R. Ionizing radiation exposure results in up-regulation of Ku70 via a p53/ataxia-telangiectasia-mutated protein-dependent mechanism. J Biol Chem, 275: 6651-6656, 2000.
Brown, W. T. Genetic diseases of premature aging as models of senescence. Annu Rev Gerontol Geriatr, 10: 23-42, 1990.
Brown, W. T. Premature aging syndromes. Curr Probl Dermatol, 17: 152-165, 1987.
Campisi J 2002 Cancer and aging: yin, yang, and p53. Sci Aging Knowledge Environ 2002:pe1
Campisi J 2004 Fragile fugue: p53 in aging, cancer and IGF signaling. Nat Med 10:231-2
Campisi J 2005 Aging, tumor suppression and cancer: high wire-act! Mech Ageing Dev 126:51-8
Campisi, J. Senescent cells, tumor suppression, and organismal aging: good citizens, bad neighbors. Cell, 120: 513-522, 2005.

S Chang, C., Da Silva, S. L., Ideta, R., Lee, Y., Yeh, S., and Burbach, J. P. Human and rat TR4 orphan receptors specify a subclass of the steroid receptor superfamily. Proc Natl Acad Sci USA, 91: 6040-6044, 1994.
Chen L, Huang S, Lee L, et al. 2003 WRN, the protein deficient in Werner syndrome, plays a critical structural role in optimizing DNA repair. Aging Cell 2:191-9
Chen Y, Lee W H, Chew H K 1999 Emerging roles of BRCA1 in transcriptional regulation and DNA repair. J Cell Physiol 181:385-92
Chen Y T, Collins L L, Uno H, Chang C 2005 Deficits in motor coordination with aberrant cerebellar development in mice lacking testicular orphan nuclear receptor 4. Mol Cell Biol 25:2722-32
Chen Z, Trotman L C, Shaffer D, et al. 2005 Crucial role of p53-dependent cellular senescence in suppression of Pten-deficient tumorigenesis. Nature 436:725-30
Claiborne, A. Catalase activity. Boca Raton, Fla.: CRS Press, 1985.
Collado M, Gil J, Efeyan A, et al. 2005 Tumour biology: senescence in premalignant tumours. Nature 436:642
Collins, L. L., Lee, Y. F., Heinlein, C. A., Liu, N. C., Chen, Y. T., Shyr, C. R., Meshul, C. K., Uno, H., Platt, K. A.; and Chang, C. Growth retardation and abnormal maternal behavior in mice lacking testicular orphan nuclear receptor 4. Proc Natl Acad Sci USA, 101: 15058-15063, 2004.
de Boer, J., Andressoo, J. O., de Wit, J., Huijmans, J., Beems, R. B., van Steeg, H., Weeda, G., van der Horst, G. T., van Leeuwen, W., Themmen, A. P., Meradji, M., and Hoeijmakers, J. H. Premature aging in mice deficient in DNA repair and transcription. Science, 296: 1276-1279, 2002.
Deng C X, Brodie S G 2000 Roles of BRCA1 and its interacting proteins. Bioessays 22:728-37
DePinho R A 2000 The age of cancer. Nature 408:248-54
Difilippantonio M J, Zhu J, Chen H T, et al. 2000 DNA repair protein Ku80 suppresses chromosomal aberrations and malignant transformation. Nature 404:510-4
Douglas, P., Moorhead, G. B., Ye, R., and Lees-Miller, S. P. Protein phosphatases regulate DNA-dependent protein kinase activity. J Biol Chem, 276: 18992-18998, 2001.
Droge, W. Oxidative stress and aging. Adv Exp Med Biol, 543: 191-200, 2003.
Epstein C J, Motulsky A G 1996 Werner syndrome: entering the helicase era. Bioessays 18:1025-7
Espejel S, Martin M, Klatt P, Martin-Caballero J, Flores J M, Blasco MA 2004 Shorter telomeres, accelerated ageing and increased lymphoma in DNA-PKcs-deficient mice. EMBO Rep 5:503-9
Fairbairn D W, Olive P L, O'Neill K L 1995 The comet assay: a comprehensive review. Mutat Res 339:37-59
Farmand, F., Ehdaie, A., Roberts, C. K., and Sindhu, R. K. Lead-induced dysregulation of superoxide dismutases, catalase, glutathione peroxidase, and guanylate cyclase. Environ Res, 98: 33-39, 2005.
Feinberg A P, Vogelstein B 1983 A technique for radiolabeling DNA restriction endonuclease fragments to high specific activity. Anal Biochem 132:6-13
Finkel T, Holbrook N J 2000 Oxidants, oxidative stress and the biology of ageing. Nature 408:239-247
Gazdar AF 2007 DNA repair and survival in lung cancer—the two faces of Janus. N Engl J Med 356:771-773
Genova, M. L., Pich, M. M., Bemacchia, A., Bianchi, C., Biondi, A., Bovina, C., Falasca, A. I., Formiggini, G., Castelli, G. P., and Lenaz, G. The mitochondrial production of reactive oxygen species in relation to aging and pathology. Ann N Y Acad Sci, 1011: 86-100, 2004.
Greenwald B D, Harpaz N, Yin J, et al. 1992 Loss of heterozygosity affecting the p53, Rb, and mcc/apc tumor suppressor gene loci in dysplastic and cancerous ulcerative colitis. Cancer Res 52:741-5
Grenon M, Gilbert C, Lowndes NF 2001 Checkpoint activation in response to double-strand breaks requires the Mre11/Rad50/Xrs2 complex. Nat Cell Biol 3:844-7
Guarente, L. and Picard, F. Calorie restriction—the SIR2 connection. Cell, 120: 473-482, 2005.
Haaf, T., Raderschall, E., Reddy, G., Ward, D. C., Radding, C. M., and Golub, E. I. Sequestration of mammalian Rad51-recombination protein into micronuclei. J Cell Biol, 144: 11-20, 1999.
Hasty, P., Campisi, J., Hoeijmakers, J., van Steeg, H., and Vijg, J. Aging and genome maintenance: lessons from the mouse? Science, 299: 1355-1359, 2003.
Havre P A, Rice M, Ramos R, Kmiec E B 2000 HsRec2/Rad51L1, a protein influencing cell cycle progression, has protein kinase activity. Exp Cell Res 254:33-44
Huang Y, Nakada S, Ishiko T, et al. 1999 Role for caspase-mediated cleavage of Rad51 in induction of apoptosis by DNA damage. Mol Cell Biol 19:2986-97
Huang, H. and Manton, K. G. The role of oxidative damage in mitochondria during aging: a review. Front Biosci, 9: 1100-1117, 2004.
Iyengar R 2005 Teaching resources. Introduction: Overview of pathways and networks and GPCR signaling. Sci STKE 2005:tr4
Jacobs, H. T. The mitochondrial theory of aging: dead or alive? Aging Cell, 2: 11-17, 2003.
Jornot, L., Petersen, H., and Junod, A. F. Hydrogen peroxide-induced DNA damage is independent of nuclear calcium but dependent on redox-active ions. Biochem J, 335 (Pt 1): 85-94, 1998.
Kasamon K M, Dawson N A 2004 Update on hormone-refractory prostate cancer. Curr Opin Urol 14:185-193
Kim, E., Xie, S., Yeh, S. D., Lee, Y. F., Collins, L. L., Hu, Y. C., Shyr, C. R., Mu, X. M., Liu, N. C., Chen, Y. T., Wang, P. H., and Chang, C. Disruption of TR4 orphan nuclear receptor reduces the expression of liver apolipoprotein E/C-I/C-II gene cluster. J Biol Chem, 278: 46919-46926, 2003.
Kirkwood, T. B. Understanding the odd science of aging. Cell, 120: 437-447, 2005.
Kouzarides T 2000 Acetylation: a regulatory modification to rival phosphorylation? Embo J 19:1176-1179
Kreisberg J I, Malik S N, Prihoda T J, et al. 2004 Phosphorylation of Akt (Ser473) is an excellent predictor of poor clinical outcome in prostate cancer. Cancer Res 64:5232-6
Krejci L, Damborsky J, Thomsen B, Duno M, Bendixen C 2001 Molecular dissection of interactions between Rad51 and members of the recombination-repair group. Mol Cell Biol 21:966-76
Krtolica A, Parrinello S, Lockett S, Desprez P Y, Campisi J 2001 Senescent fibroblasts promote epithelial cell growth and tumorigenesis: a link between cancer and aging. Proc Natl Acad Sci USA 98:12072-7
Kurimasa, A., Kumano, S., Boubnov, N. V., Story, M. D., Tung, C. S., Peterson, S. R., and Chen, D. J. Requirement for the kinase activity of human DNA-dependent protein kinase catalytic subunit in DNA strand break rejoining. Mol Cell Biol, 19: 3877-3884, 1999.
Kuro-o, M., Matsumura, Y., Aizawa, H., Kawaguchi, H., Suga, T., Utsugi, T., Ohyama, Y., Kurabayashi, M., Kaname, T., Kume, E., Iwasaki, H., Iida, A., Shiraki-Iida, T., Nishikawa, S., Nagai, R., and Nabeshima, Y. I. Mutation of the mouse klotho gene leads to a syndrome resembling ageing. Nature, 390: 45-51, 1997.
Kurumizaka H, Ikawa S, Nakada M, et al. 2001 Homologous-pairing activity of the human DNA-repair proteins Xrcc3.Rad51C. Proc Natl Acad Sci USA 98:5538-43
Land, H., Parada, L. F., and Weinberg, R. A. Tumorigenic conversion of primary embryo fibroblasts requires at least two cooperating oncogenes. Nature, 304: 596-602, 1983.
Lee, H. J., Lee, Y., Burbach, J. P., and Chang, C. Suppression of gene expression on the simian virus 40 major late promoter by human TR4 orphan receptor. A member of the steroid receptor superfamily. J Biol Chem, 270: 30129-30133, 1995.
Lee, Y. F., Shyr, C. R., Thin, T. H., Lin, W. J., and Chang, C. Convergence of two repressors through heterodimer formation of androgen receptor and testicular orphan receptor-4: a unique signaling pathway in the steroid receptor superfamily. Proc Natl Acad Sci USA, 96: 14724-14729, 1999.
Lee, Y. F., Young, W. J., Burbach, J. P., and Chang, C. Negative feedback control of the retinoid-retinoic acid/retinoid X receptor pathway by the human TR4 orphan receptor, a member of the steroid receptor superfamily. J Biol Chem, 273: 13437-13443, 1998.
Lee, Y. F., Young, W. J., Lin, W. J., Shyr, C. R., and Chang, C. Differential regulation of direct repeat 3 vitamin D3 and direct repeat 4 thyroid hormone signaling pathways by the human TR4 orphan receptor. J Biol Chem, 274: 16198-16205, 1999.
Lehmann, A. Ageing: repair and transcription keep us from premature ageing. Curr Biol, 12: R550-551, 2002.
Levitt N C, Hickson I D 2002 Caretaker tumour suppressor genes that defend genome integrity. Trends Mol Med 8:179-86
Lips J, Kaina B 2001 DNA double-strand breaks trigger apoptosis in p53-deficient fibroblasts. Carcinogenesis 22:579-85
Little J B1994 Failla Memorial Lecture. Changing views of cellular radiosensitivity. Radiat Res 140:299-311
Liu Y, Kulesz-Martin M 2001 p53 protein at the hub of cellular DNA damage response pathways through sequence-specific and non-sequence-specific DNA binding. Carcinogenesis 22:851-60
Livingstone L R, White A, Sprouse 3, Livanos E, Jacks T, Tlsty TD 1992 Altered cell cycle arrest and gene amplification potential accompany loss of wild-type p53. Cell 70:923-35
Mandavilli, B. S., Santos, J. H., and Van Houten, B. Mitochondrial DNA repair and aging. Mutat Res, 509: 127-151, 2002.
Marmorstein L Y, Ouchi T, Aaronson S A 1998 The BRCA2 gene product functionally interacts with p53 and RAD51. Proc Natl Acad Sci USA 95:13869-74
Martin, G. M. Syndromes of accelerated aging. Natl Cancer Inst Monogr, 60: 241-247, 1982.
McBride A E, Silver P A 2001 State of the arg: protein methylation at arginine comes of age. Cell 106:5-8
McCullagh P N, J. A, 1989 Generalized Linear Models: Boca Raton: Chapman & Hall.b Mekeel K L, Tang W, Kachnic L A, Luo C M, DeFrank J S, Powell S N1997 Inactivation of p53 results in high rates of homologous recombination. Oncogene 14:1847-57
Michalogou C, Vredeveld L C, Soengas M S, et al. 2005 BRAFE600-associated senescence-like cell cycle arrest of human naevi. Nature 436:720-4
Mimeault M, Batra SK 2006 Recent advances on multiple tumorigenic cascades involved in prostatic cancer progression and targeting therapies. Carcinogenesis 27:1-22
Morrison C, Sonoda E, Takao N, Shinohara A, Yamamoto K, Takeda S 2000 The controlling role of ATM in homologous recombinational repair of DNA damage. Embo J 19:463-71
Moynahan M E, Cui T Y, Jasin M 2001 Homology-directed dna repair, mitomycin-c resistance, and chromosome stability is restored with correction of a Brca1 mutation. Cancer Res 61:4842-50
Mu X, Lee Y F, Liu N C, et al. 2004 Targeted inactivation of testicular nuclear orphan receptor 4 delays and disrupts late meiotic prophase and subsequent meiotic divisions of spermatogenesis. Mol Cell Biol 24:5887-99
Nakayama H 2002 RecQ family helicases: roles as tumor suppressor proteins. Oncogene 21:9008-21
Neumeister P, Albanese C, Balent B, Greally J, Pestell R G 2002 Senescence and epigenetic dysregulation in cancer. Int J Biochem Cell Biol 34:1475-90
North P, Ganesh A, Thacker J 1990 The rejoining of double-strand breaks in DNA by human cell extracts. Nucleic Acids Res 18:6205-10
Olive P L 1999 DNA damage and repair in individual cells: applications of the comet assay in radiobiology. Int J Radiat Biol 75:395-405
O'Regan P, Wilson C, Townsend S, Thacker J 2001 XRCC2 is a nuclear RAD51-like protein required for damage-dependent RAD51 focus formation without the need for ATP binding. J Biol Chem 276:22148-53
Pandey A, Liu X, Dixon J E, Di Fiore P P, Dixit V M 1996 Direct association between the Ret receptor tyrosine kinase and the Src homology 2-containing adapter protein Grb7. J Biol Chem 271:10607-10
Parrinello S, Coppe J P, Krtolica A, Campisi J 2005 Stromal-epithelial interactions in aging and cancer: senescent fibroblasts alter epithelial cell differentiation. J Cell Sci 118:485-96
Parrinello, S., Samper, E., Krtolica, A., Goldstein, J., Melov, S., and Campisi, J. Oxygen sensitivity severely limits the replicative lifespan of murine fibroblasts. Nat Cell Biol, 5: 741-747, 2003.
Pawson T 1995 Protein modules and signalling networks. Nature 373:573-580
Peterson S R, Stackhouse M, Waltman M J, Chen F, Sato K, Chen D J 1997 Characterization of two DNA double-stranded break repair-deficient cell lines that express inactive DNA-dependent protein kinase catalytic subunits. J Biol Chem 272:10227-31
Rangarajan, A., Hong, S. J., Gifford, A., and Weinberg, R. A. Species- and cell type-specific requirements for cellular transformation. Cancer Cell, 6: 171-183, 2004.
Roubertoux, P. L., Sluyter, F., Carlier, M., Marcet, B., Maarouf-Veray, F., Cherif, C., Marican, C., Arrechi, P., Godin, F., Jamon, M., Verrier, B., and Cohen-Salmon, C. Mitochondrial DNA modifies cognition in interaction with the nuclear genome and age in mice. Nat Genet, 35: 65-69, 2003.
Salles-Passador I, Fotedar A, Fotedar R 1999 Cellular response to DNA damage. Link between p53 and DNA-PK. C R Acad Sci III 322:113-20
Schmitt C A, Fridman J S, Yang M, et al. 2002 A senescence program controlled by p53 and p16INK4a contributes to the outcome of cancer therapy. Cell 109:335-46
Scully R, Chen J, Plug A, et al. 1997 Association of BRCA1 with Rad51 in mitotic and meiotic cells. Cell 88:265-75
Seluanov A, Mittelman D, Pereira-Smith O M, Wilson J H, Gorbunova V 2004 DNA end joining becomes less efficient and more error-prone during cellular senescence. Proc Natl Acad Sci USA 101:7624-9
Serrano M, Lin A W, McCurrach M E, Beach D, Lowe S W 1997 Oncogenic ras provokes premature cell senescence associated with accumulation of p53 and p16INK4a. Cell 88:593-602
Serrano, M., Lin, A. W., McCurrach, M. E., Beach, D., and Lowe, S. W. Oncogenic ras provokes premature cell senescence associated with accumulation of p53 and p16INK4a. Cell, 88: 593-602, 1997.
Sharpless N E, DePinho R A 2005 Cancer: crime and punishment. Nature 436:636-7
Shiloh Y, Kastan MB 2001 ATM: genome stability, neuronal development, and cancer cross paths. Adv Cancer Res 83:209-54
Shyr C R, Hu Y C, Kim E, Chang C 2002 Modulation of estrogen receptor-mediated transactivation by orphan receptor TR4 in MCF-7 cells. J Biol Chem 277:14622-8
Stevnsner, T., Thorslund, T., de Souza-Pinto, N. C., and Bohr, V. A. Mitochondrial repair of 8-oxoguanine and changes with aging. Exp Gerontol, 37: 1189-1196, 2002.
Strefford J C, Foot N J, Chaplin T, et al. 2001 The characterisation of the lymphoma cell line U937, using comparative genomic hybridisation and multi-plex FISH. Cytogenet Cell Genet. 94:9-14
Sturzbecher H W, Donzelmann B, Henning W, Knippschild U, Buchhop S 1996 p53 is linked directly to homologous recombination processes via RAD51/RecA protein interaction. Embo J 15:1992-2002
Sun, L. Q., Lee, D. W., Zhang, Q., Xiao, W., Raabe, E. H., Meeker, A., Miao, D., Huso D. L., and Arceci, R. J. Growth retardation and premature aging phenotypes in mice with disruption of the SNF2-like gene, PAST. Genes Dev, 18: 1035-1046, 2004.
Sung P 1997 Function of yeast Rad52 protein as a mediator between replication protein A and the Rad51 recombinase. J Biol Chem 272:28194-7
Takata M, Sasaki M S, Sonoda E, et al. 1998 Homologous recombination and non-homologous end-joining pathways of DNA double-strand break repair have overlapping roles in the maintenance of chromosomal integrity in vertebrate cells. Embo J 17:5497-508
Tang W, Willers H, Powell S N 1999 p53 directly enhances rejoining of DNA double-strand breaks with cohesive ends in gamma-irradiated mouse fibroblasts. Cancer Res 59:2562-5
Tashiro S, Walter J, Shinohara A, Kamada N, Cremer T 2000 Rad51 accumulation at sites of DNA damage and in postreplicative chromatin. J Cell Biol 150:283-91
Thacker J, Chalk J, Ganesh A, North P 1992 A mechanism for deletion formation in DNA by human cell extracts: the involvement of short sequence repeats. Nucleic Acids Res 20:6183-8
Thompson L H, Schild D 2001 Homologous recombinational repair of DNA ensures mammalian chromosome stability. Mutat Res 477:131-53
Tran H, Brunet A, Grenier J M, et al. 2002 DNA repair pathway stimulated by the forkhead transcription factor FOXO3a through the Gadd45 protein. Science 296:530-4
Trifinovic, A., Wredenberg, A., Falkenberg, M., Spelbrink, J. N., Rovio, A. T., Bruder, C. E., Bohlooly, Y. M., Gidlof, S., Oldfors, A., Wibom, R., Tomell, J., Jacobs, H. T., and Larsson, N. G. Premature ageing in mice expressing defective mitochondrial DNA polymerase. Nature, 429: 417-423, 2004.
Tyner, S. D., Venkatachalam, S., Choi, J., Jones, S., Ghebranious, N., Igelmann, H., Lu, X., Soron, G., Cooper, B., Brayton, C., Hee Park, S., Thompson, T., Karsenty, G., Bradley, A., and Donehower, L. A. p53 mutant mice that display early ageing-associated phenotypes. Nature, 415: 45-53, 2002.
Vogel, H., Lim, D. S., Karsenty, G., Finegold, M., and Hasty, P. Deletion of Ku86 causes early onset of senescence in mice. Proc Natl Acad Sci USA, 96: 10770-10775, 1999.
Wei, Y. H. and Lee, H. C. Oxidative stress, mitochondrial DNA mutation, and impairment of antioxidant enzymes in aging. Exp Biol Med (Maywood), 227: 671-682, 2002.
Weinberg, R. A. Oncogenes and the molecular biology of cancer. J Cell Biol, 97: 1661-1662, 1983.
Wiesmuller L, Cammenga J, Deppert W W 1996 In vivo assay of p53 function in homologous recombination between simian virus 40 chromosomes. J Virol 70:737-44
Xu X, Qiao W, Linke S P, et al. 2001 Genetic interactions between tumor suppressors Brca1 and p53 in apoptosis, cell cycle and tumorigenesis. Nat Genet. 28:266-71-71
Yokoyama, T., Date, C., Kokubo, Y., Yoshiike, N., Matsumura, Y., and Tanaka, H. Serum vitamin C concentration was inversely associated with subsequent 20-year incidence of stroke in a Japanese rural community. The Shibata study. Stroke, 31: 2287-2294, 2000.
Young, W. J., Lee, Y. F., Smith, S. M., and Chang, C. A bidirectional regulation between the TR2/TR4 orphan receptors (TR2/TR4) and the ciliary neurotrophic factor (CNTF) signaling pathway. J Biol Chem, 273: 20877-20885, 1998.
Young, W. J., Smith, S. M., and Chang, C. Induction of the intronic enhancer of the human ciliary neurotrophic factor receptor (CNTFRalpha) gene by the TR4 orphan receptor. A member of steroid receptor superfamily. J Biol Chem, 272: 3109-3116, 1997.
Yu C E, Oshima J, Fu Y H, Wijsman E M, Hisama F, Alisch R, Matthews S, Nakura J, Miki T, Ouais S, Martin G M, Mulligan J, Schellenberg G D 1996 Positional cloning of the Werner's syndrome gene. Science 272:258-262
Zachara, B. A., Pawluk, H., Korenkiewicz, J., and Skok, Z. Selenium levels in kidney, liver and heart of newborns and infants. Early Hum Dev, 63: 103-111, 2001.
Zheng Z, Chen T, Li X, Haura E, Sharma A, Bepler G 2007 DNA synthesis and repair genes RRM1 and ERCC1 in lung cancer. N Engl J Med 356:800-808
Zhivotovsky B, Kroemer G 2004 Apoptosis and genomic instability. Nat Rev Mol Cell Biol 5:752-62

What is claimed is:

1. A method of treating a cancer in a subject comprising administering to the subject a composition comprising a low molecular weight antioxidant (LMWA) and TR4.

2. The method of claim 1, wherein the LMWA is selected from the group of LMWA consisting of Vitamin E, Vitamin C, selenium, Niacin, Vitamin A, and superoxide dismutase.

3. The method of claim 2, wherein the LMWA is vitamin E.

4. The method of claim 1, wherein the TR4 is encoded by a TR4 gene.

5. The method of claim 4, wherein the TR4 gene is encoded on a vector.

6. The method of claim 4, wherein the TR4 gene is operably linked to a promoter containing a stress responsive element (SRE).

7. The method of claim 1, further comprising administering an agent that phosphorylates TR4 at the serine at position 144 (S144) of TR4.

8. The method of claim 7, wherein the agent also dephosphorylates TR4 at the serine at position 351 (S351) of TR4.

9. The method of claim 1, further comprising administering an agent that dephosphorylates TR4 at the serine at position 351 (S351) of TR4.

10. The method of claim 1, wherein the cancer can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

11. The method of claim 10, wherein the cancer is prostate cancer.

12. A method of diagnosing the presence of a cancer in a subject comprising obtaining a tissue sample from the subject, and measuring the level of TR4 in the cytoplasm and nucleus of the sample, wherein the diagnosis of cancer increases with the increase of TR4 in the cytoplasm.

13. A method of assessing the severity of a cancer in a subject comprising obtaining a tissue sample from the subject, and measuring the level of TR4 in the cytoplasm and nucleus of the sample, wherein the severity of the cancer increases with the increase of TR4 in the cytoplasm.

14. A method of assessing progression of a cancer in a subject comprising obtaining a tissue sample from the subject, and measuring the level of TR4 in the cytoplasm and nucleus of the sample, wherein the severity of the cancer increases with the increase of TR4 in the cytoplasm.

15. (canceled)

16. A method of screening for an agent that inhibits DNA damage comprising administering the agent to a cell and measuring the activity of TR4, wherein a increase in TR4 activity relative to a control indicates an agent that inhibits DNA damage.

17-27. (canceled)

28. A method of screening for an agent that inhibits a cancer comprising administering the agent to a cell and measuring the activity of TR4, wherein a increase in TR4 activity relative to a control indicates an agent that inhibits cancer.

29-38. (canceled)

39. A method of screening for an agent that inhibits DNA damage comprising administering the agent to a cell and measuring the activity of TR4, wherein a increase in TR4 activity relative to a control indicates an agent that inhibits DNA damage.

40. A method of inhibiting DNA damage in a subject comprising administering to the subject a composition comprising TR4.

41-43. (canceled)

44. A method of treating an inflammatory condition in a subject comprising administering to the subject a composition comprising TR4.

45-48. (canceled)

49. A vector comprising a TR4 gene.

50. (canceled)

51. A composition comprising the vector of claim 49.

52-54. (canceled)

55. A cell comprising the vector of claim 49.

56. A method of treating a disease in a subject comprising administering to the subject the vector of claim 49.

57-60. (canceled)

61. A method of modulating Vitamin E uptake in a subject comprising administering to the subject a vector comprising TR4.

62. A composition comprising an agent that phosphorylates TR4 at S144, a low molecular weigh antioxidant, and a vector comprising a TR4 gene.

63. A method of increasing the efficacy of radiation treatment for a subject comprising administering to the subject an agent that inhibits TR4.

64-65. (canceled)

66. A method of treating a disease related to premature aging in a subject comprising administering to the subject a composition comprising TR4.

67-71. (canceled)

72. A composition comprising a low molecular weight antioxidant (LMWA) and TR4.

73-80. (canceled)


SEQUENCE LISTING (9 sequences)

SEQ ID NO: 1 - protein, 180 aa, Artificial Sequence
  Description of Artificial Sequence note = synthetic construct
GRIQIVTDSASVERLLGKADVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASRAFDTLAKALNTTDSASPPSLADGIDASGGGSIHVISRDQSTPIIEVEGPLLSDTHVTFK

SEQ ID NO: 2 - protein, 180 aa, Artificial Sequence; identical to SEQ ID NO: 1
  Description of Artificial Sequence note = synthetic construct

SEQ ID NO: 3 - protein, 180 aa, Artificial Sequence
  Description of Artificial Sequence note = synthetic construct
GRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASRAFDTLAKALNTTDSSSSPSLADGIDTSGGGSIHVISRDQSTPIIEVEGPLLSDTHVTFK

SEQ ID NO: 4 - protein, 180 aa, Artificial Sequence
  Description of Artificial Sequence note = synthetic construct
GRIQIVTDSASVERLLGKTDVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASRAFDTLAKALNTTDSSSSPSLADGIDASGGGSIHVISRDQSTPIIEVEGPLLSDTHVTFK

SEQ ID NO: 5 - protein, 180 aa, Artificial Sequence
  Description of Artificial Sequence note = synthetic construct
GRIQIVTDSASVERLLGKADVQRAQVVEYCVVCGDKASGRHYGAVSCEGCKGFFKRSVRKNLTYSCRSNQDCIINKHHRNRCQFCRLKKCLEMGMKMESVQSERKPFDVQREKPTNCAASRAFDTLAKALSTTDGTAVPNLADGMDPTGGGNIHVISRDQSTPIIEVEGPLLTDTHVTFK

SEQ ID NO: 6 - protein, 166 aa, Artificial Sequence
  Description of Artificial Sequence note = synthetic construct
PHLQLLTENSPDQGPNKVFDLCVVCGDKASGRHYGAITCEGCKGFFKRSIRKNLVYSCRGSKDCVINKHHRNRCQYCRLQRCIAFGMKQDSVQCERKPIEVSREKSSNCAASRAFDTLAKALTPGESTSCQSPEEGMEGSPHLIAGEPSFVEKEGPLLSESHVAFR

SEQ ID NO: 7 - protein, 168 aa, Artificial Sequence
  Description of Artificial Sequence note = synthetic construct
QHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASRAFDTLAKALNPGESTACQSSVAGMEGSVHLITGDSSINYTEKEGPLLSDSHVAFR

SEQ ID NO: 8 - DNA, 18 bp, Artificial Sequence
  Description of Artificial Sequence note = synthetic construct
ggtagccagcctgtcttc

SEQ ID NO: 9 - DNA, 20 bp, Artificial Sequence
  Description of Artificial Sequence note = synthetic construct
cctcctcttccttccatagc